SEQUENCE LISTING (2 sequences)

SEQ ID NO: 1 - protein, 124 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, synthetic construct [sequence omitted]

(19) United States
(12) Patent Application Publication
(10) Pub. No.: US 2026/0176351 A1
(43) Pub. Date: Jun. 25, 2026

(54) FIXED SEED TRAIN SCHEDULE FOR IMPROVED PROTEIN PRODUCTION

(71) Applicant: Regeneron Pharmaceuticals, Inc., Tarrytown, NY (US)

(72) Inventors: Sarah O’Donovan, Cork (IE); Niall Harnett, Limerick (IE)

(21) Appl. No.: 19/424,822

(22) Filed: Dec. 18, 2025

Related U.S. Application Data
(60) Provisional application No. 63/736,748, filed on Dec. 20, 2024.
(60) Provisional application No. 63/752,379, filed on Jan. 31, 2025.

(51) Int. Cl. C07K 16/24

(52) U.S. Cl. CPC C07K 16/247; C07K 2317/14

(57) ABSTRACT

The present invention generally pertains to methods of cultivating cells and producing a recombinant protein of interest, such as dupilumab. In particular, the present invention pertains to the use of a fixed seed schedule to determine the expansion time of seed train unit operations for improved cell growth and protein titer. The protein produced by the fixed seed schedule, such as dupilumab, can be later subjected to lipase activity reduction methods.

### CROSS-REFENCE TO RELATED APPLICATIONS

This application claims the benefit of U.S. Provisional Patent Application No. 63/942,206, filed on Dec. 16, 2025, U.S. Provisional Patent Application No. 63/752,379, filed on Jan. 31, 2025 and U.S. Provisional Patent Application No. 63/736,748, filed on Dec. 20, 2024 each of which is incorporated herein by reference in its entirety.

### SEQUENCE LISTING

The instant application contains a Sequence Listing which has been filed electronically in .xml format and is hereby incorporated by reference in its entirety. Said .xml copy, created on Dec. 16, 2025, is named 070816-06153.xml and is 2,814 bytes in size BACKGROUND

In biologics manufacturing, optimizing titer and protein production in production bioreactors includes optimizing cell culture conditions and cell growth in preceding seed train bioreactors. In order to maximize initial viable cell density (VCD) in a production bioreactor while maintaining cell culture health, previous methods relied on target VCDs in seed train bioreactors to determine cell culture expansion times. However, variability in cell growth can lead to inconsistencies in manufacturing scheduling. Thus, a need exists for biologics manufacturing systems and methods that optimize protein titer and promote metabolically favorable cell culture performance while maintaining consistent operations timing.

### SUMMARY

Methods have been developed for improved cultivation of cells and production of recombinant proteins. A fixed seed train schedule was developed using an analysis of optimized viable cell densities for producing a high protein titer. Use of the fixed seed train schedule (also referred to herein as the “fixed seed schedule”) allowed for a consistent manufacturing schedule and led to a higher starting viable cell density in the final (N-1) vessel of a seed train, resulting in reduced expansion time in the N-1 vessel. The fixed seed schedule further provided for an increased protein titer from a production bioreactor.

This disclosure provides methods for producing a recombinant protein. In some exemplary aspects, the methods can comprise (a) culturing cells expressing a recombinant protein in a seed train; (b) transferring cells from each vessel of said seed train to the next vessel at a predetermined time point; (c) transferring cells from the final vessel of said seed train to a production bioreactor at a predetermined time point; and (d) isolating the recombinant protein from the production bioreactor. In particular, a predetermined time point is determined prior to culturing the cells and is independent of the features of the cell culture to which it is applied. It should be understood that the vessels of a seed train may conventionally be referred to with reference to their order before the end of the seed train; for example, the final vessel of the seed train may be referred to as N-1, the preceding vessel may be referred to as N-2, and so forth.

In one aspect, the recombinant protein is selected from a group consisting of an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, a variant thereof, a fragment thereof, and a multimer thereof. In another aspect, the recombinant protein is dupilumab.

In one aspect, the final vessel is a 3,000 L bioreactor.

In one aspect, the predetermined time point of step (c) is from 60 hours to 80 hours, from 65 hours to 75 hours, from 65 hours to 72.5 hours, from 67 hours to 70 hours, from 68 hours to 69 hours, or about 68.5 hours between inoculation of said final vessel and inoculation of said production bioreactor. In a specific aspect, the predetermined time point is about 68.5 hours.

In one aspect, the seed train comprises a 500 L bioreactor at N-2. In a specific aspect, a predetermined time point for said 500 L bioreactor is from 50 to 52 hours or about 51 hours between inoculation of said 500 L bioreactor and inoculation of the next vessel in the seed train.

In one aspect, the seed train comprises a 50 L bioreactor at N-3. In a specific aspect, a predetermined time point for said 50 L bioreactor is from 73 to 75 hours or about 74 hours between inoculation of said 50 L bioreactor and inoculation of the next vessel in the seed train.

In one aspect, the seed train comprises a 20 L bioreactor at N-4. In a specific aspect, a predetermined time point for said 20 L bioreactor is from 73 to 75 hours or about 74 hours between inoculation of said 20 L bioreactor and inoculation of the next vessel in the seed train.

In one aspect, the seed train comprises a 2 L bioreactor at N-5. In a specific aspect, a predetermined time point for said 2 L bioreactor is from 47.5 to 48.5 hours or about 48 hours between inoculation of said 2 L bioreactor and inoculation of the next vessel in the seed train.

In one aspect, the seed train comprises a 500 mL shake flask at N-6. In a specific aspect, a predetermined time point for said 500 mL shake flask is from 79 to 80.5 hours or about 80 hours between inoculation of said 500 mL shake flask and inoculation of the next vessel in the seed train

In one aspect, the production bioreactor is a 10,000 L bioreactor.

In one aspect, a titer of said recombinant protein is increased compared to a titer of a recombinant protein produced using a method wherein cells from each vessel of said seed train are transferred to the next vessel at a variable time point.

In one aspect, the cells are selected from the group consisting of CHO cells, HEK293 cells, and BHK cells. In a specific aspect, the CHO cells are selected from the group consisting of CHO-K1, CHO DUX B-11, Veggie-CHO, GS-CHO, S-CHO, or CHO lec.

In one aspect, the cells of step (c) have a viable cell density from 40-50×10⁵ viable cells/mL, from 45-50×10⁵ viable cells/mL, from 46-48×10⁵ viable cells/mL, from 46.5-47.5×10⁵ viable cells/mL, from 46.9-47.1×10⁵ viable cells/mL, about 40×10⁵ viable cells/mL, about 45×10⁵ viable cells/mL, about 47×10⁵ viable cells/mL, or about 50×10⁵ viable cells/mL.

In another aspect, the method further comprises determining said predetermined time points by selecting time points for each vessel of said seed train based on the achievement of optimized viable cell densities for each vessel of said seed train, wherein optimized viable cell densities are optimized for increased protein production from a production bioreactor.

The inventions further provide as aspects as set forth below. The inventions provide methods of culturing cells that produce recombinant proteins, such as dupilumab, using a seed train, wherein the method comprises the steps of: culturing cells in a first bioreactor (N-5) for a predetermined period of time; transferring the cells from the first bioreactor to a second bioreactor (N-4) and culturing the cells for a predetermined period of time; transferring the cells from the second bioreactor to a third bioreactor (N-3) and culturing the cells for a predetermined period of time; transferring the cells from the third bioreactor to a fourth bioreactor (N-2) and culturing the cells for a predetermined period of time; and transferring the cells from the fourth bioreactor to a fifth bioreactor (N-1) and culturing the cells for a predetermined period of time, wherein the cells express the recombinant protein, and the cells can be transferred to a production bioreactor. The steps can include culturing cells in a shake flask (N-6) for a predetermined period of time, and transferring the cells to the first bioreactor.

The first bioreactor can be a 2 L bioreactor and the predetermined period of time can be 47.5 to 48.5 hours. The first bioreactor can be a 2 L bioreactor and the predetermined period of time can be 48 hours. The second bioreactor can be a 20 L bioreactor and the predetermined period of time can be 73 to 75 hours. The second bioreactor can be a 20 L bioreactor and the predetermined period of time can be 74 hours. The third bioreactor can be a 50 L bioreactor and the predetermined period of time can be 73 to 75 hours. The third bioreactor can be a 50 L bioreactor and the predetermined period of time can be 74 hours. The fourth bioreactor can be a 500 L bioreactor and the predetermined period of time can be 50 to 52 hours. The fourth bioreactor can be a 500 L bioreactor and the predetermined period of time can be 51 hours. The fifth bioreactor can be a 3000 L bioreactor and the predetermined period of time can be 65 to 72.5 hours. The fifth bioreactor can be a 3000 L bioreactor and the predetermined period of time can be 68.5 hours. The shake flask can be a 500 mL shake flask and the predetermined period of time can be 79 to 80.5 hours. The shake flask can be a 500 mL shake flask and the predetermined period of time can be 80 hours.

The methods can achieve a viable cell density selected from the group consisting of 40-50×10⁵ viable cells/mL, 45-50×10⁵ viable cells/mL, 46-48×10⁵ viable cells/mL, 46.5-47.5×10⁵ viable cells/mL, 46.9-47.1×10⁵ viable cells/mL, about 40×10⁵ viable cells/mL, about 45×10⁵ viable cells/mL, about 47×10⁵ viable cells/mL, and about 50×10⁵ viable cells/mL. The production bioreactor can be a 10,000 L bioreactor.

The inventions provide increased production of dupilimab per 10,000 L working volume as compared to a method based upon target viable cell density, wherein the increase can be selected from the group of ranges consisting of 0.1 to 2.5 kg, 0.5 to 2.5 kg, 0.5 to 2 kg, 0.6 to 2 kg, 0.6 to 1.9 kg, 0.7 to 1.8 kg, 0.7 to 1.7 kg, 0.7 to 1.6 kg, 0.8 to 1.6 kg, 0.8 to 1.5 kg, 0.9 to 1.5 kg, 0.9 to 1.4 kg. 0.9 to 1.3 kg, 1 to 1.5 kg, 1 to 1.4 kg, 1 to 1.3 kg, 1 to 1.2 kg, 1 to 1.1 kg, and 1.1 to 1.2 kg. An increased average of 1.1 kg of dupilumab per 10,000 L working volume as compared to a method based upon target viable cell density can be provided.

The inventions further provide methods of culturing cells that produce dupilumab, wherein the method comprises the steps of: culturing cells in a 500 mL shake flask (N-6) for 79 to 80.5 hours; transferring the cells from the 500 mL shake flask to a 2 L bioreactor (N-5) and culturing the cells for 47.5 to 48.5 hours; transferring the cells from the 2 L bioreactor to a 20 L bioreactor (N-4) and culturing the cells for 73 to 75 hours; transferring the cells from the 20 L bioreactor to a 50 L bioreactor (N-3) and culturing the cells for 73 to 75 hours; transferring the cells from the 50 L bioreactor to a 500 L bioreactor (N-2) and culturing the cells for 50 to 52 hours; transferring the cells from the 500 L bioreactor to a 3000 L bioreactor (N-1) and culturing the cells for 65 to 72.5 hours; and transferring the cells from the 3000 L bioreactor to a production bioreactor.

The 500 mL shake flask (N-6) can be cultured for 80 hours. The 2 L bioreactor can be cultured for 48 hours. The 20 L bioreactor can be cultured for 74 hours. The 50 L bioreactor can be cultured for 74 hours. The 500 L bioreactor can be cultured for 51 hours. The 3000 L bioreactor can be cultured for 68.5 hours.

The methods provide increased production of dupilimab per 10,000 L working volume as compared to a method based upon target viable cell density, wherein the increase can be selected from the group of ranges consisting of 0.1 to 2.5 kg, 0.5 to 2.5 kg, 0.5 to 2 kg, 0.6 to 2 kg, 0.6 to 1.9 kg, 0.7 to 1.8 kg, 0.7 to 1.7 kg, 0.7 to 1.6 kg, 0.8 to 1.6 kg, 0.8 to 1.5 kg, 0.9 to 1.5 kg, 0.9 to 1.4 kg. 0.9 to 1.3 kg, 1 to 1.5 kg, 1 to 1.4 kg, 1 to 1.3 kg, 1 to 1.2 kg, 1 to 1.1 kg, and 1.1 to 1.2 kg. An increased average of 1.1 kg of dupilumab per 10,000 L working volume as compared to a method based upon target viable cell density also can be provided. Seed trains that performs the methods are provided. Dupilumab produced by the methods are provided. Dupilumab produced by the seed trains also are provided.

These culturing methods and proteins produced hereby can be optionally combined with lipase activity reducing methods disclosed in WO 2023/215750 and PCT/US2024/054106, which are hereby incorporated by reference, and exemplified below.

The inventions further provide methods of reducing lipase activity in a pharmaceutical composition comprising dupilumab, wherein the method comprises subjecting dupilumab produced by the culturing methods set forth above and a lipase to anion exchange chromatography, wherein a pH of the dupilumab loaded to the anion exchange chromatography column can be between about 7.8 and about 8.3. The inventions further provide methods of reducing lipase activity in a pharmaceutical composition comprising dupilumab, wherein the method comprises subjecting dupilumab produced according to the culturing methods set forth above and a lipase to stress conditions to form a sample with inactivated lipase; and formulating the sample with inactivated lipase to produce a pharmaceutical composition comprising dupilimab with reduced lipase activity. The formulating step comprises adding a fatty acid ester to the sample, wherein the fatty acid ester can be polysorbate 20, polysorbate 40, polysorbate 60, or polysorbate 80. Fatty acid esters can be polysorbate 80 and a concentration of oleic acid esters in the polysorbate 80 can be at least 80%. A concentration of oleic acid esters in the polysorbate 80 can be at least 98% or at least 99%. Stress conditions can include agitation stress and/or heat stress. Agitation stress can comprise shaking the sample at from 50 to 500 rpm, from 200 to 300 rpm, about 50 rpm, about 75 rpm, about 100 rpm, about 125 rpm, about 150 rpm, about 200 rpm, about 225 rpm, about 250 rpm, about 275 rpm, about 300 rpm, about 325 rpm, about 350 rpm, about 375 rpm, about 400 rpm, about 425 rpm, about 450 rpm, about 475 rpm, or about 500 rpm. Agitation stress can comprise shaking the sample for from 1 to 96 hours, from 24 to 48 hours, about 1 hour, about 2 hours, about 3 hours, about 4 hours, about 5 hours, about 6 hours, about 12 hours, about 18 hours, about 24 hours, about 30 hours, about 36 hours, about 42 hours, about 48 hours, about 60 hours, about 72 hours, about 84 hours, or about 96 hours. Heat stress can comprise storing the sample at from about 30° C. to about 60° C., from about 35° C. to about 55° C., from about 40° C. to about 50° C., from about 44° C. to about 46° C., about 30° C., about 35° C., about 40° C., about 45° C., about 50° C., about 55° C., or about 60° C. Storing for heat stress can be from 1 day to 6 months, from 3 days to 3 months, from 1 week to 2 months, from 0.5 months to 1 month, about 1 day, about 2 days, about 3 days, about 1 week, about 2 weeks, about 0.5 months, about 3 weeks, about 4 weeks, about 1 month, about 5 weeks, about 6 weeks, about 7 weeks, about 8 weeks, about 2 months, about 3 months, about 3.5 months, about 4 months, about 4.5 months, about 5 months, about 5.5 months, or about 6 months. The methods can further comprise subjecting the sample with inactivated lipase to filtration, enrichment, or chromatographic separation to remove high molecular weight (HMW) species. Chromatographic separation can comprise cation exchange chromatography. Chromatographic separation can comprise size exclusion chromatography. Formulating steps can comprise adding excipients to the sample.

The inventions further provide methods for producing a pharmaceutical composition comprising dupilumab, wherein the pharmaceutical composition has reduced lipase activity and the method comprises the steps of: (a) subjecting dupilumab produced according to the culturing methods set forth above to affinity chromatography; (b) subjecting dupilumab pooled from eluate of step (a) to viral inactivation at a pH from about 3 to about 4 and then adjusting the pH to from about 5 to about 8; (c) subjecting dupilumab pooled from step (b) to anion exchange chromatography in flowthrough mode; (d) subjecting dupilumab pooled from eluate of step (c) to hydrophobic interaction chromatography in flowthrough mode; (e) subjecting dupilumab pooled from flowthrough fractions of step (d) to virus retentive filtration; and (f) subjecting a sample from step (e) including dupilumab and a lipase to agitation stress or heat stress to form a pharmaceutical composition with reduced lipase activity.

The inventions further provide methods for producing a pharmaceutical composition comprising dupilumab, wherein the pharmaceutical composition has reduced lipase activity and the method comprises the steps of: (a) subjecting harvested dupilumab produced according to the culturing methods set forth above to affinity chromatography; (b) subjecting the dupilumab pooled from eluate of step (a) to viral inactivation at a pH from about 3 to about 4 and then adjusting the pH to from about 5 to about 8; (c) subjecting the dupilumab pooled from step (b) to anion exchange chromatography in flowthrough mode; (d) subjecting the dupilumab pooled from flowthrough fractions of step (c) to virus retentive filtration; (e) subjecting a sample from step (d) including dupilumab and a lipase to agitation stress or heat stress to form a pharmaceutical composition with reduced lipase activity. The methods can include subjecting dupilumab to filtration, enrichment, or chromatographic separation to remove high molecular weight species. Chromatographic separation can comprise ion exchange chromatography, cation exchange chromatography, or size exclusion chromatography. The pH of the sample loaded to the anion exchange (AEX) column can be between about 7.8 and about 8.3.

The inventions further provide methods for producing a dupilumab pharmaceutical composition with increased stability, comprising: reducing lipase activity in a composition by subjecting dupilumab produced according the culturing methods set forth above and a lipase to anion exchange (AEX) chromatography, wherein a pH of the sample loaded to the AEX column can be between about 7.8 and about 8.3. The methods can further comprise subjecting dupilumab after AEX chromatography to agitation stress or heat stress to reduce lipase activity. The methods can further comprise the addition of poloxamer 188 or PEG3350 to produce a formulation comprising a dupilumab pharmaceutical composition with reduced lipase activity and increased stability. The formulation can be substantially free of polysorbate. The concentration of the poloxamer 188 can be from 0.005% to 5%, from 0.05% to 2.5%, from 0.1% to 1%, about 0.05%, about 0.01%, about 0.02%, about 0.03%, about 0.04%, about 0.05%, about 0.06%, about 0.07%, about 0.08%, about 0.09%, about 0.1%, about 0.15%, about 0.2%, about 0.25%, about 0.3%, about 0.4%, about 0.5%, about 1%, about 1.5%, about 2%, about 2.5%, about 3%, about 3.5%, about 4%, about 4.5%, or about 5%. The concentration of the PEG3350 can be from 0.005% to 5%, from 0.05% to 2.5%, from 0.1% to 1%, about 0.05%, about 0.01%, about 0.02%, about 0.03%, about 0.04%, about 0.05%, about 0.06%, about 0.07%, about 0.08%, about 0.09%, about 0.1%, about 0.15%, about 0.2%, about 0.25%, about 0.3%, about 0.4%, about 0.5%, about 1%, about 1.5%, about 2%, about 2.5%, about 3%, about 3.5%, about 4%, about 4.5%, or about 5%.

The inventions further provide methods for producing a dupilumab pharmaceutical composition with increased stability, comprising: (a) subjecting harvested dupilumab according to the culturing methods set forth above to anion exchange chromatography in flowthrough mode; (b) subjecting flowthrough fractions from step (a) to hydrophobic interaction chromatography in flowthrough mode; and (c) formulating dupilumab isolated from step (b) with polysorbate 80, wherein a concentration of oleic acid esters in the polysorbate 80 can be at least 80%. The concentration of the oleic acid esters can be at least 98% or at least 99%. The pH of the dupilumab produced according to the culturing methods set forth above that is loaded to the anion exchange chromatography column can be from about 7.8 to about 8.3, from about 7.9 to about 8.2, about 7.8, about 7.9, about 8.0, about 8.1, about 8.2, or about 8.3. Dupilumab preparations made by the culturing methods and lipase activity reduction methods set forth above also are provided.

These, and other, aspects of the invention will be better appreciated and understood when considered in conjunction with the following description and accompanying drawings. The following description, while indicating various aspects and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1A shows initial viable cell density (VCD) in a 3,000 L seed train bioreactor using a variable seed schedule compared to a fixed seed schedule, according to an exemplary aspect.

FIG. 1B shows final VCD in a 3,000 L seed train bioreactor using a variable seed schedule compared to a fixed seed schedule, according to an exemplary aspect.

FIG. 2A shows initial viability in a 3,000 L seed train bioreactor using a variable seed schedule compared to a fixed seed schedule, according to an exemplary aspect.

FIG. 2B shows final viability in a 3,000 L seed train bioreactor using a variable seed schedule compared to a fixed seed schedule, according to an exemplary aspect.

FIG. 3 shows expansion time in a 3,000 L seed train bioreactor using a variable seed schedule compared to a fixed seed schedule, according to an exemplary aspect.

FIG. 4A shows initial glucose concentration in a 3,000 L seed train bioreactor using a variable seed schedule compared to a fixed seed schedule, according to an exemplary aspect.

FIG. 4B shows final glucose concentration in a 3,000 L seed train bioreactor using a variable seed schedule compared to a fixed seed schedule, according to an exemplary aspect.

FIG. 5A shows pH in a 3,000 L seed train bioreactor using a variable seed schedule compared to a fixed seed schedule, according to an exemplary aspect.

FIG. 5B shows final lactose concentration in a 3,000 L seed train bioreactor using a variable seed schedule compared to a fixed seed schedule, according to an exemplary aspect.

FIG. 5C shows final ammonia concentration in a 3,000 L seed train bioreactor using a variable seed schedule compared to a fixed seed schedule, according to an exemplary aspect.

FIG. 6A shows initial VCD in a 10,000 L production bioreactor using a variable seed schedule compared to a fixed seed schedule, according to an exemplary aspect.

FIG. 6B shows initial viability in a 10,000 L production bioreactor using a variable seed schedule compared to a fixed seed schedule, according to an exemplary aspect.

FIG. 7A shows initial glucose concentration in a 10,000 L production bioreactor using a variable seed schedule compared to a fixed seed schedule, according to an exemplary aspect.

FIG. 7B shows initial lactate concentration in a 10,000 L production bioreactor using a variable seed schedule compared to a fixed seed schedule, according to an exemplary aspect.

FIG. 7C shows initial ammonia concentration in a 10,000 L production bioreactor using a variable seed schedule compared to a fixed seed schedule, according to an exemplary aspect.

FIG. 7D shows initial potassium concentration in a 10,000 L production bioreactor using a variable seed schedule compared to a fixed seed schedule, according to an exemplary aspect.

FIG. 8A shows final glucose concentration in a 10,000 L production bioreactor using a variable seed schedule compared to a fixed seed schedule, according to an exemplary aspect.

FIG. 8B shows final lactate concentration in a 10,000 L production bioreactor using a variable seed schedule compared to a fixed seed schedule, according to an exemplary aspect.

FIG. 8C shows final ammonia concentration in a 10,000 L production bioreactor using a variable seed schedule compared to a fixed seed schedule, according to an exemplary aspect.

FIG. 8D shows final potassium concentration in a 10,000 L production bioreactor using a variable seed schedule compared to a fixed seed schedule, according to an exemplary aspect.

FIG. 9 shows final titer in a 10,000 L production bioreactor using a variable seed schedule compared to a fixed seed schedule, according to an exemplary aspect.

FIG. 10 shows a chromatogram showing relative amounts of different molecular species with (A) lower quality polysorbate 20 (PS20-A), (B) higher quality polysorbate 20 (PS20-B), and (C) polysorbate 80 (PS80), according to one aspect.

FIG. 11A shows the formation of particles in mAb1 formulations comprising SR-PS80 as measured by membrane microscopy, according to one aspect.

FIG. 11B shows the formation of particles in mAb1 formulations comprising HP-PS20 as measured by membrane microscopy, according to one aspect.

FIG. 11C shows the formation of particles in mAb1 formulations comprising SR-PS80 as measured by MFI, according to one aspect.

FIG. 11D shows the formation of particles in mAb1 formulations comprising HP-PS20 as measured by MFI, according to one aspect.

FIG. 12A shows recovery of polysorbate in mAb1 formulations comprising SR-PS80 as measured by CAD-UHPLC, according to one aspect.

FIG. 12B shows recovery of polysorbate in mAb1 formulations comprising HP-PS20 as measured by CAD-UHPLC, according to one aspect.

FIG. 13 shows a correlation between formation of particles and recovery of polysorbate in mAb1 formulations, according to one aspect.

FIG. 14 shows a correlation between formation of particles and recovery of polysorbate in mAb5 formulations, according to one aspect.

FIG. 15A shows the formation of particles in mAb1 formulations comprising SR-PS80 and subjected to HIC purification as measured by membrane microscopy, according to one aspect.

FIG. 15B shows the formation of particles in mAb1 formulations comprising HP-PS20 and subjected to HIC purification as measured by membrane microscopy, according to one aspect.

FIG. 15C shows the formation of particles in mAb1 formulations comprising SR-PS80 and subjected to HIC purification as measured by MFI, according to one aspect.

FIG. 15D shows the formation of particles in mAb1 formulations comprising HP-PS20 and subjected to HIC purification as measured by MFI, according to one aspect.

FIG. 16A shows recovery of polysorbate in mAb1 formulations comprising SR-PS80 and subjected to HIC purification as measured by CAD-UHPLC, according to one aspect.

FIG. 16B shows recovery of polysorbate in mAb1 formulations comprising HP-PS20 and subjected to HIC purification as measured by CAD-UHPLC, according to one aspect.

FIG. 17 shows a correlation between phospholipase activity (in parts per million) and percent degradation of polysorbate 20, according to one aspect.

FIG. 18 shows a visualization of models generated for a multivariate study of AEX risk factors and responses, according to one aspect.

FIG. 19 shows a comparison of AEX pool lipase activity (%) performance during confirmation batches compared to small-scale predictions from Monte Carlo simulations, according to one aspect.

FIG. 20 shows polysorbate recovery from formulations comprising mAb5, mAb6 and mAb7, according to one aspect.

FIG. 21 shows polysorbate recovery from mAb5 formulations at varying concentrations and incubation times, according to one aspect.

FIG. 22 shows homology modeling depicting surface hydrophobicity of mAb5, mAb6 and mAb7, according to one aspect.

FIG. 23 shows a number of subvisible particulates (≥10 μm) measured by the membrane miscroscopic method, in protein drug products comprising various types of polysorbate 80, according to one aspect.

FIG. 24 shows a number of subvisible particulates (≥10 μm) measured by micro-flow imaging (MFI), in protein drug products comprising various types of polysorbate 80, according to one aspect.

FIG. 25 shows a chemical structure of polyoxyethylene (20) sorbitan monooleate, the predominant fatty acid ester in polysorbate 80, according to one aspect.

FIG. 26 shows a measured concentration of free fatty acids in protein drug products comprising various types of polysorbate 80, according to one aspect.

FIG. 27A illustrates a polysorbate structure and main degradation routes, according to one aspect.

FIG. 27B illustrates a structure of poloxamer 188, according to one aspect.

FIG. 28 shows surfactant recovery for mAb5 formulations comprising PS20, PS80, or poloxamer 188 (P188) at various temperatures, according to one aspect.

FIG. 29 shows a number of particles ≥10 μm (upper panel) and ≥25 μm (lower panel) identified by membrane microscopy in a formulation of 150 mg/mL of an anti-IL-4R antibody containing a lipase and either PEG3350 or poloxamer 188 (at concentrations of 0.02%, 0.04%, or 0.1% w/v) and stored at 5° C. for up to 36 months, according to one aspect.

FIG. 30A shows an impact of PEG3350 concentrations on the stability (as a percentage of HMW species measured by SE-UPLC) of a formulation of 150 mg/mL of an anti-IL-4R antibody against agitation stress at room temperature for a period of from 30-120 minutes.

FIG. 30B shows an impact of poloxamer 188 concentrations on the stability (as a percentage of HMW species measured by SE-UPLC) of a formulation of 150 mg/mL of an anti-IL-4R antibody against agitation stress at room temperature for a period of from 30-120 minutes.

FIG. 31 shows an impact of polysorbate 20, polysorbate 80, PEG3350, and poloxamer 188 (at varying concentrations) on the stability (as a percentage of HMW species measured by SE-UPLC) of a formulation of 150 mg/mL of an anti-IL-4R antibody against thermal stress (45° C.) for a period of up to 56 days.

### DETAILED DESCRIPTION

In biologics manufacturing and development, a high titer and protein production is desirable and critically depends on the design and operation of the bioreactors used for cell growth and protein production. The production bioreactor (PBR) is a stainless-steel vessel with capacities ranging from 2,000 L-10,000 L where lots are processed. The cell culture is initially processed through series of small flasks, wave bags and bioreactors, which are used as seed to inoculate the PBR. These small bioreactors used in operations are collectively referred to as the “seed train” and facilitate the growth of cells through the log phase of the growth curve. The viable cell density (VCD) results from the seed train, particularly the conventionally used 3,000 L seed train bioreactor, are crucial in upstream operations, as this culture is used as inoculum for the 10,000 L production bioreactor with the aim of producing a high final titer of the desired protein.

Key parameters useful for evaluating the performance of the 3,000 L seed train bioreactor include metabolite levels, specifically glucose and lactate, VCD, and O₂ sparge levels. A previous strategy used for determining expansion times of cell cultures throughout the seed train was based on targeting final VCD requirements before transferring to the subsequent bioreactors, ensuring a consistent initial VCD in the next unit operation. This strategy led to variation in operation times and resulted in irregularities in manufacturing scheduling.

In order to address this issue, a novel strategy was developed using a fixed seed schedule in order to streamline the manufacturing schedule while maintaining the final VCD in the 3000 L, N-1 bioreactor, at desirable levels, within previous historical experience. The two strategies were compared by performing statistical analysis of key cell culture parameters, and the new fixed seed schedule was found to allow for a more consistent manufacturing schedule while maintaining 3,000 L N-1 bioreactor final VCDs at the desired target level. Some differences were found in the levels of various metabolites and parameters between the two strategies. Several parameters assessed could be helpful in maintaining healthy cell culture and optimizing productive performance of the manufacturing process. Importantly, in addition to reducing expansion times and producing a consistent manufacturing schedule, a fixed seed schedule resulted in significantly improved protein titer from a 10,000 L bioreactor compared to a variable seed train schedule (also referred to as a “variable seed schedule”).

These and other aspects of the invention are set forth in further detail below.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, bispecific antibodies, and antigen-binding proteins.

Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated by reference). In some aspects, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain aspects, the recombinant protein can be an antibody, for example, a chimeric, humanized, or fully human antibody. In certain aspects, the recombinant protein can be an antibody of an isotype selected from group consisting of: IgG, IgM, IgA1, IgA2, IgD, or IgE. In certain aspects the antibody molecule is a full-length antibody (e.g., an IgG1) or alternatively the antibody can be a fragment (e.g., an Fc fragment or a Fab fragment).

The term “antibody,” as used herein, is generally intended to refer to immunoglobulin molecules comprising four polypeptide chains, two heavy (H) chains and two light (L) chains inter-connected by disulfide bonds, as well as multimers thereof (e.g., IgM); however, immunoglobulin molecules consisting of only heavy chains (i.e., lacking light chains) are also encompassed within the definition of the term “antibody.” Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementary determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

In some aspects, the protein of interest is a human antibody. The term “human antibody,” as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies of the disclosure may, in some aspects, be recombinant human antibodies. The term “recombinant human antibody,” as used herein, is intended to include all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain aspects, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some aspects, an antibody fragment comprises a sufficient amino acid sequence of the parent antibody of which it is a fragment that it binds to the same antigen as does the parent antibody; in some aspects, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entire teachings of which are herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein, “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the system and method disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

An “isolated antibody,” as used herein, is intended to refer to an antibody that is substantially free of other antibodies having different antigenic specificities (e.g., an isolated antibody that specifically binds hIL-4Rα (human interleukin-4 receptor alpha) is substantially free of antibodies that specifically bind antigens other than hIL-4Rα).

The term “specifically binds,” or the like, means that an antibody or antigen-binding fragment thereof forms a complex with an antigen that is relatively stable under physiologic conditions. Specific binding can be characterized by a dissociation constant of at least about 1×10⁻⁶ M or greater. Methods for determining whether two molecules specifically bind are well known in the art and include, for example, equilibrium dialysis, surface plasmon resonance, and the like. An isolated antibody that specifically binds hIL-4Rα may, however, have cross-reactivity to other antigens, such as IL-4Rα molecules from other species (orthologs). In the context of the present disclosure, multispecific (e.g., bispecific) antibodies that bind to hIL-4Rα as well as one or more additional antigens are deemed to “specifically bind” hIL-4Rα. Moreover, an isolated antibody may be substantially free of other cellular material and/or chemicals. However, in some instances, the isolated antibody may be copurified with a phospholipase expressed by a mammalian cell line (e.g., CHO cells) from which the anti-IL-4R antibody is produced.

For example, for antibody production, aspects of the inventions are amenable for research and production use for diagnostics and therapeutics based on all major antibody classes, namely IgG, IgA, IgM, IgD, and IgE. IgG is a preferred class, and includes subclasses IgG1 (including IgG1k and IgG1κ), IgG2, IgG3, and IgG4. In some aspects, the protein of interest or polypeptide of interest is an antibody, a human antibody, a humanized antibody, a chimeric antibody, a monoclonal antibody, a multispecific antibody, a bispecific antibody, an antibody fragment, an antigen-binding antibody fragment, a single chain antibody, a diabody, triabody or tetrabody, a Fab fragment or a F(ab′)2 fragment, an IgD antibody, an IgE antibody, an IgM antibody, an IgG antibody, an IgG1 antibody, an IgG2 antibody, an IgG3 antibody, an IgG4 antibody, a fusion protein, a receptor fusion protein, an antibody-derived protein, or combinations thereof. In one aspect, the antibody is an IgG1 antibody. In one aspect, the antibody is an IgG2 antibody. In one aspect, the antibody is an IgG4 antibody. In one aspect, the antibody is a chimeric IgG2/IgG4 antibody. In one aspect, the antibody is a chimeric IgG2/IgG1 antibody. In one aspect, the antibody is a chimeric IgG2/IgG1/IgG4 antibody. Derivatives, components, domains, chains, and fragments of the above are also included.

In some aspects, the antibody is selected from the group consisting of an anti-Programmed Cell Death 1 antibody (e.g. an anti-PD1 antibody as described in U.S. Pat. App. Pub. No. US2015/0203579A1), an anti-Programmed Cell Death Ligand-1 antibody (e.g. an anti-PD-L1 antibody as described in in U.S. Pat. App. Pub. No. US2015/0203580A1), an anti-DII4 antibody, an anti-Angiopoietin-2 antibody (e.g. an anti-ANG2 antibody as described in U.S. Pat. No. 9,402,898), an anti-Angiopoietin-Like 3 antibody (e.g. an anti-AngPtl3 antibody as described in U.S. Pat. No. 9,018,356), an anti-platelet derived growth factor receptor antibody (e.g. an anti-PDGFR antibody as described in U.S. Pat. No. 9,265,827), an anti-Erb3 antibody, an anti-Prolactin Receptor antibody (e.g. anti-PRLR antibody as described in U.S. Pat. No. 9,302,015), an anti-Complement 5 antibody (e.g. an anti-C5 antibody as described in U.S. Pat. App. Pub. No US2015/0313194A1), an anti-TNF antibody, an anti-epidermal growth factor receptor antibody (e.g. an anti-EGFR antibody as described in U.S. Pat. No. 9,132,192 or an anti-EGFRvIII antibody as described in U.S. Pat. App. Pub. No. US2015/0259423A1), an anti-Proprotein Convertase Subtilisin Kexin-9 antibody (e.g. an anti-PCSK9 antibody as described in U.S. Pat. No. 8,062,640 or U.S. Pat. App. Pub. No. US2014/0044730A1), an anti-Growth And Differentiation Factor-8 antibody (e.g. an anti-GDF8 antibody, also known as anti-myostatin antibody, as described in U.S. Pat. No. 8,871,209 or U.S. Pat. No. 9,260,515), an anti-Glucagon Receptor (e.g. anti-GCGR antibody as described in U.S. Pat. App. Pub. Nos. US2015/0337045A1 or US2016/0075778A1), an anti-VEGF antibody, an anti-IL1R antibody, an interleukin 4 receptor antibody (e.g., an anti-IL4R antibody as described in U.S. Pat. App. Pub. No. US2014/0271681A1 or U.S. Pat. No. 8,735,095 or U.S. Pat. No. 8,945,559), an anti-interleukin 6 receptor antibody (e.g. an anti-IL6R antibody as described in U.S. Pat. Nos. 7,582,298, 8,043,617 or 9,173,880), an anti-IL1 antibody, an anti-IL2 antibody, an anti-IL3 antibody, an anti-IL4 antibody, an anti-IL5 antibody, an anti-IL6 antibody, an anti-IL7 antibody, an anti-interleukin 33 (e.g. anti-IL33 antibody as described in U.S. Pat. App. Pub. Nos. US2014/0271658A1 or US2014/0271642A1), an anti-Cluster of differentiation 3 antibody (e.g. an anti-CD3 antibody, as described in U.S. Pat. App. Pub. Nos. US2014/0088295A1 and US20150266966A1, and in U.S. Application No. 62/222,605), an anti-Cluster of differentiation 20 antibody (e.g. an anti-CD20 antibody as described in U.S. Pat. App. Pub. Nos. US2014/0088295A1 and US20150266966A1, and in U.S. Pat. No. 7,879,984), an anti-CD19 antibody, an anti-CD28 antibody, an anti-Cluster of Differentiation-48 antibody (e.g. anti-CD48 antibody as described in U.S. Pat. No. 9,228,014), an anti-Fel d1 antibody (e.g. as described in U.S. Pat. No. 9,079,948), an anti-influenza virus antibody, an anti-Respiratory syncytial virus antibody (e.g. anti-RSV antibody as described in U.S. Pat. App. Pub. No. US2014/0271653A1), an anti-Middle East Respiratory Syndrome virus antibody (e.g. an anti-MERS-CoV antibody as described in U.S. Pat. App. Pub. No. US2015/0337029A1), an anti-Ebola virus antibody (e.g. as described in U.S. Pat. App. Pub. No. US2016/0215040), an anti-Zika virus antibody, an anti-Severe Acute Respiratory Syndrome (SARS) antibody (e.g., an anti-SARS-CoV antibody), an anti-COVID-19 antibody (e.g., an anti-SARS-CoV-2 antibody), an anti-Lymphocyte Activation Gene 3 antibody (e.g. an anti-LAG3 antibody, or an anti-CD223 antibody), an anti-Nerve Growth Factor antibody (e.g. an anti-NGF antibody as described in U.S. Pat. App. Pub. No. US2016/0017029 and U.S. Pat. Nos. 8,309,088 and 9,353,176) and an anti-Activin A antibody. In some aspects, the bispecific antibody is selected from the group consisting of an anti-CD3×anti-CD20 bispecific antibody (as described in U.S. Pat. App. Pub. Nos. US2014/0088295A1 and US20150266966A1), an anti-CD3×anti-Mucin 16 bispecific antibody (e.g., an anti-CD3×anti-Muc16 bispecific antibody), an anti-CD3×BCMA bispecific antibody, and an anti-CD3×anti-Prostate-specific membrane antigen bispecific antibody (e.g., an anti-CD3×anti-PSMA bispecific antibody). See also U.S. Patent Publication No. US 2019/0285580 A1. Also included are a Met×Met antibody, an agonist antibody to NPR1, an LEPR agonist antibody, a MUC16×CD28 antibody, a GITR antibody, an IL-2Rg antibody, an EGFR×CD28 antibody, a Factor XI antibody, antibodies against SARS-CoV-2 variants, a Fel d 1 multi-antibody therapy, and a Bet v 1 multi-antibody therapy. Derivatives, components, domains, chains and fragments of the above also are included. In one aspect, the protein of interest or polypeptide of interest comprises a combination of any of the foregoing.

Cells that produce exemplary antibodies can be cultured according to the inventions. In some aspects, the protein of interest or polypeptide of interest is selected from the group consisting of Alirocumab, Atoltivimab, Maftivimab, Odesivimab, Odesivimab-ebgn, Casirivimab, Imdevimab, Cemplimab and Cemplimab-rwlc (human IgG4 monoclonal antibody that binds to PD-1), Sarilumab, Fasinumab, Nesvacumab, Dupilumab (human monoclonal antibody of the IgG4 subclass that binds to the IL-4R alpha (α) subunit and thereby inhibits Interleukin 4 (IL-4) and Interleukin 13 (IL-13) signaling), Trevogrumab, Evinacumab, Evinacumab-dgnb, Fianlimab, Garetosmab, Itepekimab, Odrononextamab, Pozelimab, Rinucumab, and modifications, truncations, and variations thereof.

Additional exemplary antibodies include Ravulizumab-cwvz, Abciximab, Adalimumab, Adalimumab-atto, Ado-trastuzumab, Alemtuzumab, Atezolizumab, Avelumab, Basiliximab, Belimumab, Benralizumab, Bevacizumab, Bezlotoxumab, Blinatumomab, Brentuximab vedotin, Brodalumab, Canakinumab, Capromab pendetide, Certolizumab pegol, Cetuximab, Denosumab, Dinutuximab, Durvalumab, Eculizumab, Elotuzumab, Emicizumab-kxwh, Emtansine alirocumab, Evolocumab, Golimumab, Guselkumab, Ibritumomab tiuxetan, Idarucizumab, Infliximab, Infliximab-abda, Infliximab-dyyb, Ipilimumab, Ixekizumab, Mepolizumab, Necitumumab, Nivolumab, Obiltoxaximab, Obinutuzumab, Ocrelizumab, Ofatumumab, Olaratumab, Omalizumab, Panitumumab, Pembrolizumab, Pertuzumab, Ramucirumab, Ranibizumab, Raxibacumab, Reslizumab, Rinucumab, Rituximab, Secukinumab, Siltuximab, Tocilizumab, Trastuzumab, Ustekinumab, and Vedolizumab.

Dupilumab (Dupixent®) is a monoclonal antibody developed as a collaboration between Regeneron and Sanofi, approved in the United States by the Food and Drug Administration in March 2017 as the first antibody-based treatment of atopic dermatitis in adults (Thibodeaux et al., 2019, Hum. Vaccines & Immunother., 15:2129-2139; Rodrigues et al., 2019, G. Ital. Dermatol. Venereol., 154). Atopic dermatitis (AD) is a chronic inflammatory skin disorder affecting up to 20% of the worldwide population, characterized by xerotic, erythematous, and lichenified papules and plaques. Dupilumab can be used along with topical corticosteroids, or as the sole treatment (D'Ippolito and Pisano, 2018, Pharmacy and Therapeutics, 43(9):532).

In October 2018, Dupixent® was approved for the treatment of moderate-to-severe asthma with the eosinophilic phenotype for patients aged 12 and older as an add-on maintenance therapy aimed to suppress the atopic conditions and improve patient life quality by reducing symptoms and morbidity (Thibodeaux et al.). Dupixent® has also been approved for the treatment AD in children over 6 months old, asthma of the eosinophilic phenotype or when oral corticosteroid-dependent in children over 6 years old, eosinophilic esophagitis (EoE) in patients over the age of 12, prurigo nodularis (PN) in adults, and as an add-on treatment for chronic rhinosinusitis with nasal polyposis (CRSwNP) in adults (Patient Information—Dupixent® (Dupilumab) injection for subcutaneous use, Regeneron, regeneron.com/downloads/dupixent_ppi.pdf, (accessed 25 May 2023); Take Action with DUPIXENT® (Dupilumab), dupixent.com, (accessed 24 Jul. 2023)).

Dupilumab is a fully human IgG4 monoclonal antibody with a molecular weight of approximately 147 kDa and is produced using Chinese hamster ovary (CHO) cell suspension culture (D'Ippolito and Pisano; Patient Information). The antibody binds to the IL-4Rα subunits of Type 1 and Type 2 IL-4 receptors, inhibiting the IL-4 and IL-13 signaling pathways. This reduces the release of cytokines and chemokines, which are inflammatory mediators, as well as the release of nitric oxide and IgE, and leads to an increase in IL-4 and IL-13 serum levels. IL-4 and IL-13 play a key role in type 2 inflammation, which is integral to atopic diseases such as asthma, atopic dermatitis, or chronic sinusitis with nasal polyposis. IL-4 induces naïve CD4+ T cells to differentiate into Th2 effector cells, and IL-13 is involved in goblet cell metaplasia, smooth muscle alterations, fibrosis, mucus hypersecretion, and increased airway hyperreactivity. Additionally, both IL-4 and IL-13 promote the chemotaxis of eosinophils to inflammation sites, and class switching of B-cell immunoglobulins to IgE and IgG4 (in humans) or IgG1 (in mice) (Thibodeaux et al.; Le Floc'h et al., 2020, Allergy, 75:1188-1204).

Dupilumab has been shown to decrease FeNO (fractional exhaled nitric oxide) and the circulating concentrations of total IgE, allergen-specific IgE, eotaxin-3, periostin, and chemokine CCL17 in asthma patients, relative to placebo (Patient Information). In the case of atopic dermatitis, Dupilumab has been shown decrease the expression of genes involved in epidermal hyperplasia (MKi67 and K16) and the Th2 inflammatory response (IL-4, IL-14, CCL17, CCL18, CCL26), reducing the thickness of lesional skin, relative to placebo (Thibodeaux et al.). When used to treat chronic rhinosinusitis with nasal polyposis, Dupilumab was found to reduce polyp size and the concentration of type 2 inflammation biomarkers (eotaxin-3 and total IgE) in blood, nasal secretions, and polyp tissues, as well as rapidly improve smell in CRSwNP patients, relative to placebo (Jonstam et al., 2019, Allergy, 74:743-752).

In addition to next generation products, the inventions also are applicable to production of biosimilars. Biosimilars are defined in various ways depending on the jurisdiction, but share a common feature of comparison to a previously approved biological product in that jurisdiction, usually referred to as a “reference product.” According to the World Health Organization, a biosimilar is a biotherapeutic product similar to an already licensed reference biotherapeutic product in terms of quality, safety and efficacy, and is followed in many countries, such as the Philippines.

A biosimilar in the U.S. is currently described as (A) a biological product that is highly similar to the reference product notwithstanding minor differences in clinically inactive components; and (B) there are no clinically meaningful differences between the biological product and the reference product in terms of the safety, purity, and potency of the product. In the U.S., an interchangeable biosimilar or product may be substituted for the previous product without the intervention of the health care provider who prescribed the previous product. In the European Union, a biosimilar is a biological medicine highly similar to another biological medicine already approved in the EU (called “reference medicine”) and includes consideration of structure, biological activity, efficacy, and safety, among other things, and these guidelines also are followed by Russia. In China, a biosimilar product currently refers to biologics that contain active substances similar to the original biologic drug and is similar to the original drug in terms of quality, safety, and effectiveness, with no clinically significant differences. In Japan, a biosimilar currently is a product that has bioequivalent/quality-equivalent quality, safety, and efficacy to a reference product already approved in Japan. In India, biosimilars currently are referred to as “similar biologics,” and refer to a similar biologic product which is similar in terms of quality, safety, and efficacy to an approved reference biological product based on comparability. In Australia, a biosimilar medicine currently is a highly similar version of a reference biological medicine. In Mexico, Columbia, and Brazil, a biosimilar currently is a biotherapeutic product that is similar in terms of quality, safety, and efficacy to an already licensed reference product. In Argentina, a biosimilar currently is derived from an original product (a comparator) with which it has common features. In Singapore, a biosimilar currently is a biological therapeutic product that is similar to an existing biological product registered in Singapore in terms of physicochemical characteristics, biological activity, safety and efficacy. In Malaysia, a biosimilar currently is a new biological medicinal product developed to be similar in terms of quality, safety and efficacy to an already registered, well established medicinal product. In Canada, a biosimilar currently is a biologic drug that is highly similar to a biologic drug that was already authorized for sale. In South Africa, a biosimilar currently is a biological medicine developed to be similar to a biological medicine already approved for human use. Production of biosimilars and its synonyms under these and any revised definitions can be undertaken according to the inventions.

Manufacture of proteins via cell culture is customarily performed using a batch or fed-batch process. Early stages of inoculum growth after vial thaw include culturing cells in a seed culture. Culturing vessels include, but are not limited to, well plates, wave bags, T-flasks, shake flasks, stirred vessels, spinner flasks, hollow fiber, air lift bioreactors, and the like. A suitable cell culturing vessel is a bioreactor. A bioreactor refers to any culturing vessel that is manufactured or engineered to manipulate or control environmental conditions. Such culturing vessels are well known in the art. Typically, cells are grown at an exponential growth rate, such as in seed train bioreactors, in order to progressively increase size and/or volume of the cell population. Seed train vessels may be referred to in relation to their order before a production bioreactor; for example, the final seed train vessel may be referred to as N-1, the preceding vessel as N-2, and so forth. After cell mass is scaled up through several bioreactor stages, cells are then transferred to a production bioreactor while the cells are still in exponential growth (log phase). It is generally considered undesirable to allow cells in batch culture, for example seed culture, to go past the log phase into stationary phase. It has been recommended that cultures should be passaged while they are in log phase, before, cells, e.g. adherent cells, reach confluence due to contact inhibition or accumulation of waste products inhibits cell growth, among other reasons (Cell Culture Basics, Gibco/Invitrogen Online Handbook, www.invitrogen.com; ATCC® Animal Cell Culture Guide, atcc.org).

This disclosure sets forth improved methods for manufacturing proteins including using a fixed seed schedule for transferring cells from one seed train vessel to the next seed train vessel or the production bioreactor. In some aspects, a fixed seed schedule is determined based on the times required to ultimately achieve an optimized initial VCD in a production bioreactor, wherein an optimized initial VCD is optimized for recombinant protein quality and/or titer. An initial VCD in a production bioreactor is determined by a final VCD in an N-1 bioreactor or final seed train vessel, and therefore it should be understood that a target VCD may equivalently be optimized based on a final VCD in an N-1 bioreactor or final seed train vessel.

In some aspects, a final seed train vessel may be a 3,000 L bioreactor. In some aspects, a production bioreactor may be a 10,000 L bioreactor. In some aspects, an optimized final VCD for an N-1 bioreactor or final seed train vessel and/or an optimized initial VCD for a production bioreactor may be from 40-50×10⁵ viable cells/mL, from 45-50×10⁵ viable cells/mL, from 46-48×10⁵ viable cells/mL, from 46.5-47.5×10⁵ viable cells/mL, from 46.9-47.1×10⁵ viable cells/mL, about 40×10⁵ viable cells/mL, about 45×10⁵ viable cells/mL, about 47×10⁵ viable cells/mL, or about 50×10⁵ viable cells/mL. In some aspects, a duration between inoculation of the final seed train vessel and inoculation of the production bioreactor is predetermined to achieve an optimized VCD. It should be understood that a duration between inoculation of a first vessel and inoculation of the subsequent vessel in or after a seed train is directly related to the total expansion time in the first vessel, and directly related to a time point at which cells are transferred from the first vessel to the subsequent vessel, and therefore the terms “predetermined duration” and “predetermined time point” are both used to describe the predetermined timing within a fixed seed schedule. The term “predetermined” in reference to durations and time points within a fixed seed schedule should be understood to mean that the duration or time point is determined previously and independently of the conditions of the specific cell culture to which it is being applied, as distinguished from durations and time points that may vary depending on the conditions or progress of the specific cell culture being cultivated.

In some aspects, the predetermined duration between inoculation of the final (N-1) seed train vessel and inoculation of the production bioreactor is from 60 hours to 80 hours, from 65 hours to 75 hours, from 67 hours to 70 hours, from 68 hours to 69 hours, or about 68.5 hours. In some aspects, the predetermined duration is from 65 hours to 72.5 hours. In some aspects, the predetermined duration is about 68.5 hours. In some aspects, the predetermined duration may be a range comprising a target value plus a permissible variation. In some aspects, the permissible variation may be plus or minus about one hour, such that the predetermined duration is for example, 68.5 hours±1 hour. In some aspects, the predetermined duration may be shorter, and therefore the expansion time in the N-1 vessel may be shorter, than a duration achieved when using a variable seed schedule.

In some aspects, a seed train may include a 500 mL shake flask, a 2 L bioreactor, a 20 L bioreactor, a 50 L bioreactor, a 500 L bioreactor, and/or a 3,000 L bioreactor. Additional exemplary predetermined time points for each vessel (or unit operation) are set forth in Table 1.

Following transfer to a production bioreactor, cells are cultured for a period of time while the composition of the medium is monitored and controlled to allow production of the protein or polypeptide of interest. After a particular yield is reached or cell viability, waste accumulation or nutrient depletion determines that the culture should be terminated, the produced protein or polypeptide is isolated. In some aspects, the use of a fixed seed schedule allows for improved protein titer in a production bioreactor.

In some aspects, cells that are useful in the method of the present invention are selected from the group consisting of CHO cells, HEK293 cells, and BHK cells. In other aspects, the cells are selected from the group consisting of CHO-K1, CHO DUX B-11, Veggie-CHO, GS-CHO, S-CHO, or CHO lec. In some exemplary aspects, the cells of the present invention are CHO-K1 cells.

Following protein expression in a production bioreactor, recombinant proteins may be harvested and subjected to further downstream processing steps in order to prepare the protein for analysis and/or for use as a product. Prior to harvesting, cell cultures may be subjected to pre-treatment steps to induce flocculation or precipitation of undesired elements, for example using a modified temperature, pH, or additive. Harvesting may include, for example, separating proteins from cells and debris using centrifugation and/or filtration.

Harvested samples may then be subjected to affinity chromatography to enrich a recombinant protein of interest. Protein A affinity chromatography may be particularly useful for enriching recombinant antibodies, antibody fragments, antibody fusion proteins or proteins otherwise derived from antibodies. Following affinity chromatography, enriched samples may be transiently subjected to an acidic pH for viral inactivation. Enriched samples may be subjected to further chromatographic processes, including, for example, reverse phase liquid chromatography, ion exchange chromatography (for example, anion exchange chromatography (AEX) and/or cation exchange chromatography (CEX)), hydrophobic interaction chromatography, hydrophilic interaction chromatography, size exclusion chromatography, and/or mixed-mode chromatography. Enriched protein samples following chromatographic steps may be further subjected to, for example, virus retentive filtration, ultrafiltration, diafiltration, concentration, buffer exchange, and/or formulation with excipients.

In some exemplary aspects, a protein produced using the methods and systems of the present invention is isolated from a production bioreactor through the subsequent steps of (a) subjecting the sample including the protein to pre-treatment using an acidic pH, (b) subjecting the pre-treated sample to centrifugation to produce clarified media comprising the protein, (c) subjecting the clarified media to protein A chromatography, (d) subjecting the protein A eluate to viral inactivation, (e) subjecting the virally inactivated sample to anion exchange chromatography, (f) subjecting the sample to cation exchange chromatography, (g) subjecting the sample to hydrophobic interaction chromatography, (h) subjecting the sample to virus retentive filtration, (i) subjecting the sample to ultrafiltration and diafiltration, and (j) subjecting the sample to formulation to provide a final drug substance. It should be understood that these steps may be customized, modified, omitted, expanded upon, or rearranged by a skilled person to accommodate a particular process and protein of interest, based on common knowledge in the field, and that the methods and systems of the present invention are not limited in use to any particular downstream processing.

It is understood that the present invention is not limited to any of the aforesaid protein(s), antibody(s), monoclonal antibody(s), bispecific antibody(s), protein expression system(s), multisubunit protein(s), cell(s), vessel(s), or cell culture condition(s), and any protein(s), antibody(s), monoclonal antibody(s), bispecific antibody(s), protein expression system(s), multisubunit protein(s), cell(s), vessel(s), or cell culture condition(s) can be selected by any suitable means.

The present invention will be more fully understood by reference to the following Examples. They should not, however, be construed as limiting the scope of the invention.

### Examples

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.), but some experimental errors and deviations should be accounted for. Unless indicated otherwise, parts are parts by weight, molecular weight is average molecular weight, temperature is in degrees Centigrade, and pressure is at or near atmospheric.

### Example 1. Development of a Fixed Seed Train Schedule for Recombinant Protein Production

Manufacturing of recombinant proteins of interest, including the anti-IL4Rα antibody dupilumab, had previously been conducted using a seed train wherein the expansion time of each unit operation was determined based on the viable cell density (VCD) at each stage. To meet the needs for higher rates of production, higher VCDs were targeted compared to conventional production strategies, especially for transfer between an N-1 3,000 L bioreactor and a 10,000 L production bioreactor. However, the use of VCD as the determinant of expansion time led to variability in the seed schedule, resulting in variability in the overall manufacturing schedule. In order to further improve on this strategy, a fixed seed schedule was developed.

A historical review of lots produced using the previous variable VCD-based seed schedule was performed, and based on the optimal timing of each unit operation in the historical data, a fixed timing for each unit operation in the seed train was selected in order to construct a fixed seed schedule. Any lots that deviated from the typical manufacturing process due to equipment or process error were excluded. About 50 batches were analyzed, and average expansion times for each unit operation were assessed.

Using this analysis, target times for each unit operation, counting from inoculation of the seed train vessel of interest to inoculation of a subsequent vessel, were established. Targets were selected on the basis of optimizing VCD to be generally as high as possible, while optimizing expansion times to be generally as low as possible. Exemplary target times (or “predetermined time points” for each unit operation) for a fixed seed schedule are shown in Table 1.

TABLE 1
Target times for each unit operation in a seed train

| | Seed Train | Target Time | Exemplary Target |
|---|---|---|---|
| Seed Train Vessel | Position | Range (h) | Time (h) |
| 500 mL shake | N-6 | 79-80.5 | 80 |
| flask | | | |
| 2 L bioreactor | N-5 | 47.5-48.5 | 48 |
| 20 L bioreactor | N-4 | 73-75 | 74 |
| 50 L bioreactor | N-3 | 73-75 | 74 |
| 500 L bioreactor | N-2 | 50-52 | 51 |
| 3,000 L bioreactor | N-1 | 65-72.5 | 68.5 |

A small amount of variability, for example a target time plus or minus one hour, was allowed for each unit operation. Using the exemplary target times set forth in Table 1, a fixed seed schedule was created. The new schedule was validated using dupilumab-producing CHO cells grown in chemically defined media. Stainless steel bioreactors were employed. Twenty batches of cells were grown in a seed train, and the VCD throughout the seed train was measured to determine whether VCD levels met historical target levels at each unit operation for each batch. VCD was measured using Nova Biomedical Bioprofile® Flex2 cell culture analyzer. Data was analyzed using JMP® 17.1.1 statistical discovery software. Cells grown using the fixed seed schedule consistently met target VCDs, and therefore the fixed seed schedule was further assessed for cell culture performance in the 3,000 L N-1 seed train vessel and the 10,000 L production bioreactor.

### Example 2. A Fixed Seed Schedule Reduces Expansion Time in a 3,000 L Seed Train Bioreactor Operation

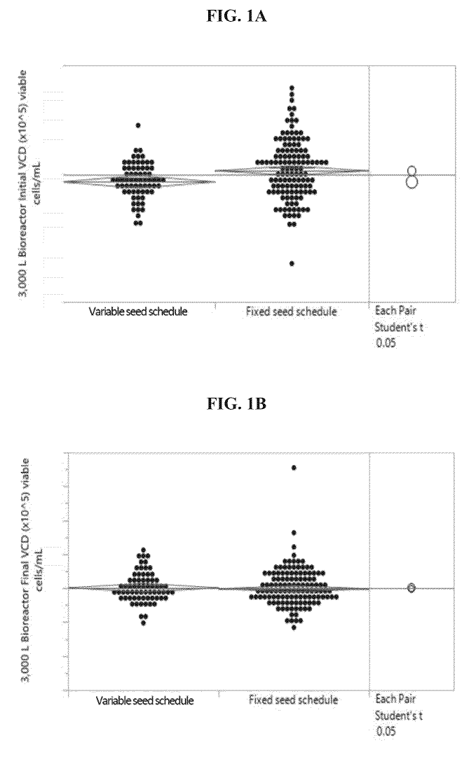

The impact of a fixed seed schedule on operational and metabolite parameters in 3,000 L seed train bioreactors, and the subsequent impact in 10,000 L production bioreactors, was investigated. Dupilumab-producing CHO cells were cultivated and assessed as described above. The VCD of the 3,000 L initial sample is an indicator of the cell biomass accumulated throughout previous seed train unit operations. FIG. 1A shows that the initial VCD in a 3,000 L seed train bioreactor is significantly higher when using a fixed seed schedule compared to the variable seed schedule. A significantly higher initial VCD using the fixed seed schedule indicates that the fixed seed schedule in earlier unit operations allows for high accumulation of biomass and high 3,000 L seed density. This results in lots from N-1 3,000 L bioreactors being processed with high VCD and in less time compared to using the variable seed schedule.

The final VCD in the N-1 3,000 L bioreactor is important in maintaining the productive performance of the culture in the production bioreactor (PBR), as this is transferred into a 10,000 L production bioreactor as starting inoculum. An excessively low VCD may lead to lower protein production due to fewer cells producing protein, while an excessively high VCD may lead to inferior cell culture performance. For optimal dupilumab production, a target final VCD for the 3,000 L seed train bioreactor and initial VCD for the 10,000 L production bioreactor is about 47×10⁵ viable cells/mL. Using a fixed seed schedule, a target time between inoculation of the 3,000 L bioreactor and inoculation of the 10,000 L bioreactor that was compatible with the target VCD was determined to be about 68.5 hours, or 68.5±1 hours. FIG. 1B shows that there are no significant differences in the final VCD in the N-1 3,000 L bioreactor between fixed the seed schedule and the variable seed schedule. As discussed above, a consideration for the fixed seed schedule is ensuring that cell densities remained within historical experience, which was achieved here.

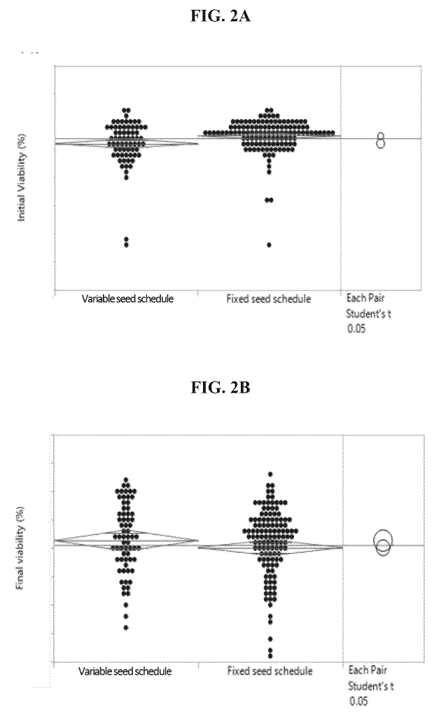

FIG. 2A shows that the initial viability in the 3,000 L seed train bioreactor is significantly higher for lots processed with the fixed seed schedule compared to the variable seed schedule. FIG. 2B shows that there is no significant difference between the two strategies when comparing the 3,000 L final viability. Therefore, the fixed seed schedule shows no negative impact on cell culture viability profile.

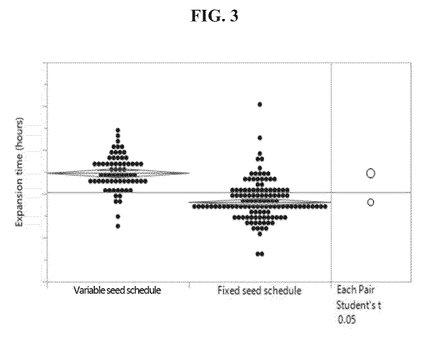

FIG. 3 shows that the expansion time on average per lot for the fixed seed schedule is significantly lower than the variable seed schedule. A key benefit contemplated in implementing the fixed seed schedule was achieving manufacturing consistency for inoculation of the 10,000 L production bioreactor. In addition to providing consistent timing, having a reduced expansion time in the 3,000 L seed train bioreactor potentially leads to more metabolically favorable cell culture conditions and improved cell culture performance. The 3,000 L seed train bioreactor was further assessed for metabolic changes in the fixed seed schedule compared to the variable seed schedule.

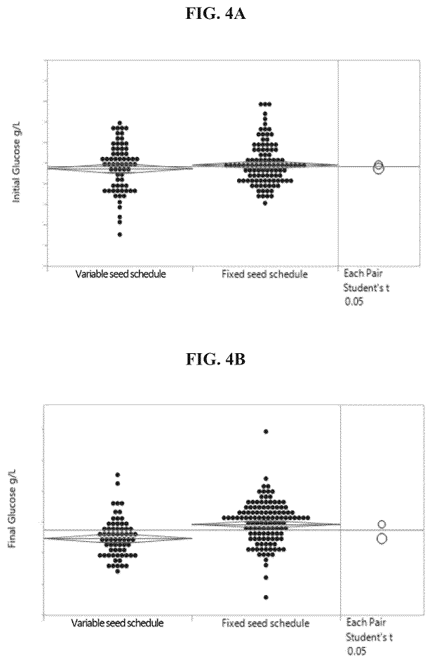

Glucose added in the form of dextrose serves as a major nutrient and energy source for the cell culture, and its concentration varies depending on the cell culture biomass in conjunction with expansion time. Glucose concentration serves as an indicator of cell growth, where low values of glucose can indicate high growth in cell biomass. FIG. 4A shows that there is no significant difference in 3,000 L initial glucose concentrations between the fixed seed schedule and variable seed schedule strategies. As the initial VCD in the 3,000 L seed train bioreactor is higher when using the fixed seed schedule, it may be expected that increased biomass earlier on in the seed train allows for reduced glucose consumption later in the seed train.

Lower concentrations of glucose are expected at the end of the stationary phase, when there is maximum consumption of available glucose in the media. FIG. 4B shows significantly higher glucose in the 3,000 L final sample for the fixed seed schedule compared to the variable seed schedule. This can be attributed to an excess of glucose that is not used by the cells, because the initial VCD when using the fixed seed schedule is already higher than the variable seed schedule, and the desired final VCD is reached in a shorter expansion time. Further, this is consistent with slightly higher initial glucose in the 3,000 L bioreactor for the fixed seed schedule.

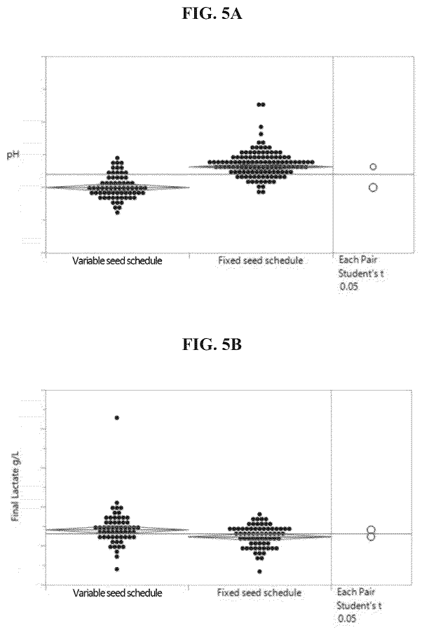

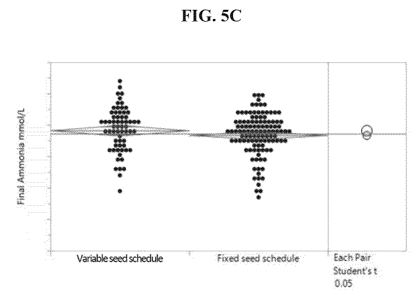

FIG. 5A shows that the culture pH is significantly higher in the 3,000 L seed train bioreactor using the fixed seed schedule compared to the variable seed schedule. Changes in pH are potentially attributable to metabolic byproducts produced during cell growth. Lactate is generated after metabolism of glucose via metabolic pathways and has a key role in lowering pH through acidosis. FIG. 5B shows significantly lower final lactate concentrations in 3,000 L seed train bioreactor using the fixed seed schedule compared to the variable seed schedule. Lower lactate is potentially favorable due to the “lactate switch,” wherein lactate consumption exceeds lactate production. Low levels of lactate help to maintain cellular stress due to pH being controlled at a favorable level, contributing to an overall healthy cell culture.

FIG. 5C shows no significant difference between the two strategies for final levels of ammonia, another metabolic byproduct.

### Example 3. A Fixed Seed Schedule Improves Protein Titer in a 10,000 L Bioreactor Operation

Production lots are transferred to production bioreactors (PBR) from N-1 3,000 L bioreactors for further processing, with an aim of entering the new bioreactor with a high VCD. The goal of the PBR is to produce the protein of interest within the specification and while ensuring that quality standards are met. In particular, a fixed seed schedule was developed that optimized production of the anti-IL4Rα antibody dupilumab.

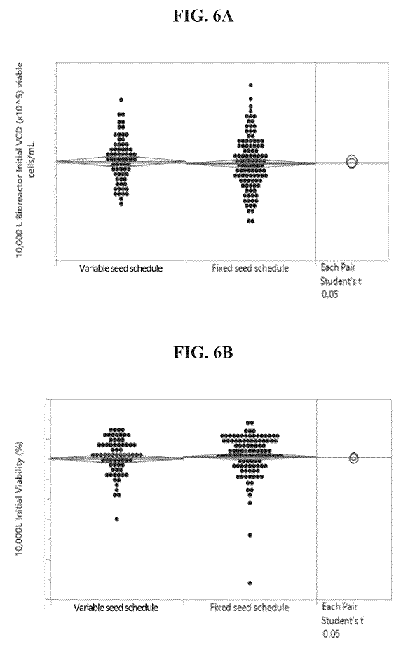

FIG. 6A shows that there was no difference in initial VCD in the 10,000 L production bioreactor between the fixed seed schedule and the variable seed schedule, and FIG. 6B shows that there was no difference in initial viability. This is to be expected, since the final VCD in the N-1 3,000 L seed train bioreactor is similar for both strategies, as shown in FIG. 1B.

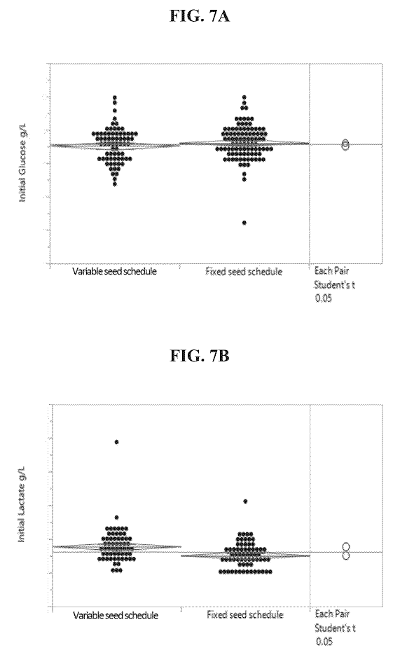

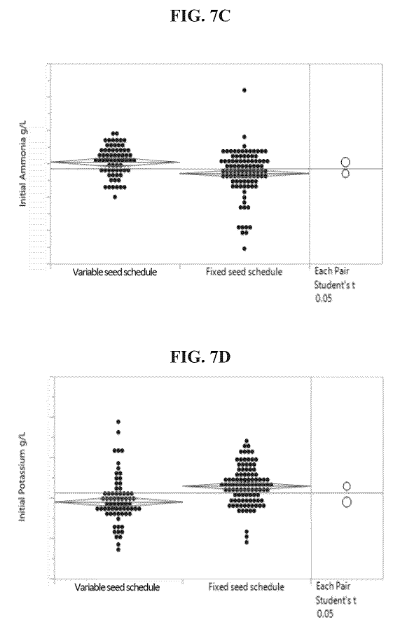

FIG. 7A shows that there was no significant difference in initial glucose concentration in the 10,000 L production bioreactor between the fixed seed schedule and the variable seed schedule. This is consistent with the results from the 3,000 L bioreactor, potentially indicating that higher cell growth earlier in the seed train grown using the fixed seed schedule results in reduced glucose consumption later in the seed train and in the production bioreactor. Further, relatively higher initial glucose levels in the 10,000 L production bioreactor using the fixed seed schedule could be expected based on the relatively higher final glucose levels in the 3,000 L seed train bioreactor.

Initial lactate and ammonia levels were significantly lower for the fixed seed schedule compared to the variable seed schedule, potentially indicating metabolically favorable conditions for cell culture performance, as shown in FIG. 7B and FIG. 7C. However, initial potassium concentration was significantly higher for the fixed seed schedule, as shown in FIG. 7D. Potassium may have a negative impact on cell culture by reducing viability, but this effect was not seen here, as shown in FIG. 6B.

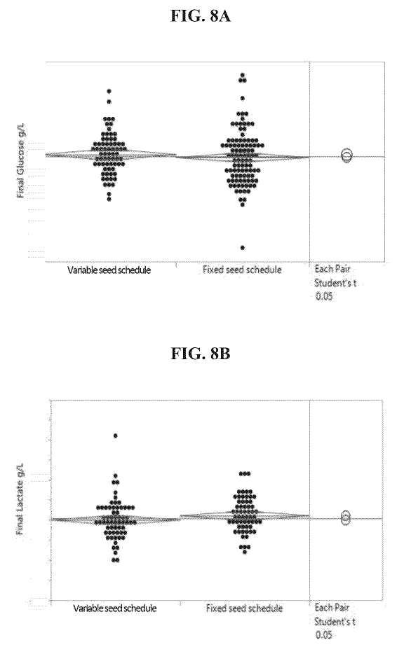

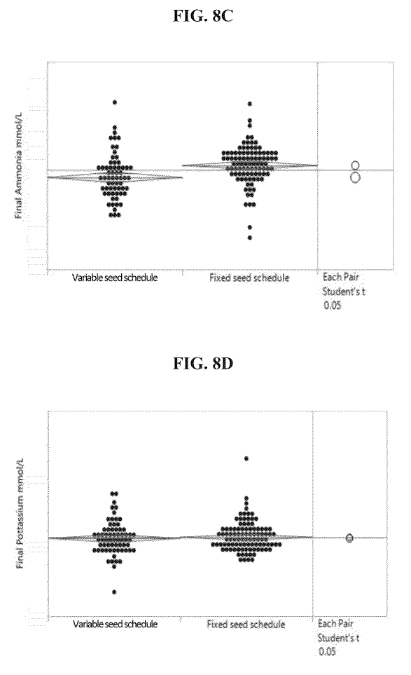

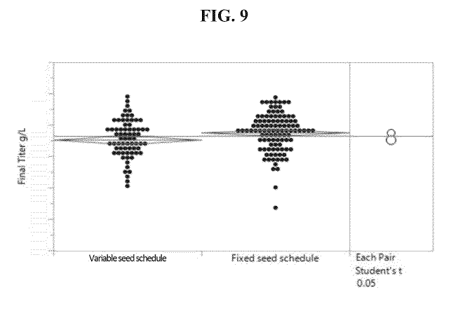

As shown in FIG. 8A, FIG. 8B, and FIG. 8D, respectively, no difference was seen in final glucose, lactate, or potassium concentrations in the 10,000 L production bioreactor between the fixed seed schedule and the variable seed schedule. Final ammonia concentrations were higher in the fixed seed schedule, as shown in FIG. 8C.

A key target of the manufacturing process is producing a high titer of a recombinant protein. For measuring protein titer, cell culture samples were taken after 10.4 days in culture in a 10,000 L production bioreactor. Samples were subjected to liquid chromatography-ultraviolet detection (LC-UV) analysis to quantify protein concentration. Surprisingly, in addition to providing a consistent manufacturing schedule, the fixed seed schedule further resulted in a significantly improved final titer of the anti-IL4Rα antibody dupilumab in a 10,000 L production bioreactor compared to the variable seed schedule, as shown in FIG. 9. The fixed feed schedule can provide increased production of dupilimab per 10,000 L working volume as compared to a method based upon target viable cell density, wherein the increase can be selected from the group of ranges consisting of 0.1 to 2.5 kg, 0.5 to 2.5 kg, 0.5 to 2 kg, 0.6 to 2 kg, 0.6 to 1.9 kg, 0.7 to 1.8 kg, 0.7 to 1.7 kg, 0.7 to 1.6 kg, 0.8 to 1.6 kg, 0.8 to 1.5 kg, 0.9 to 1.5 kg, 0.9 to 1.4 kg, 0.9 to 1.3 kg, 1 to 1.5 kg, 1 to 1.4 kg, 1 to 1.3 kg, 1 to 1.2 kg, 1 to 1.1 kg, and 1.1 to 1.2 kg. Moreover, the increased final titer resulted in an average of 1.1 kg of additional anti-IL4Rα antibody dupilumab per 10,000 L working volume.

Overall, a fixed seed schedule was developed that demonstrated the capacity to meet historical goals for VCDs, provide a consistent manufacturing schedule, and finally result in an improved higher titer for a recombinant protein, in particular dupilumab. The fixed seed schedule resulted in a higher accumulation of cell biomass in earlier seed train unit operations, reducing the growth requirements in the N-1 3,000 L seed train bioreactor and pointing to more metabolically favorable cell culture conditions for inoculation in the 10,000 L production bioreactor that led to improved performance.

The methods and systems disclosed herein can be used in further combination with additional methods and systems for culturing cells and the production of a protein of interest, such as dupilumab. In particular, the seed train methods and systems disclosed in the preceding Examples can be practiced in combination with methods and systems for improving protein stability and reducing lipase activity. Exemplary methods and systems for improving protein stability and reducing lipase activity disclosed in U.S. Pat. No. 10,342,876, U.S. Patent Application Publication No. 20190083618 A1, U.S. Provisional Application No. 63/337,532, WO 2023/215750, and PCT/US2024/054106, and as set forth below.

### Example 4. Materials and Methods for Reduction of Lipase Activity

### Determination of Subvisible Particles

For the determination of subvisible particles, suitable methods include “Method 1” (Light Obscuration Particle Count Test) and “Method 2” (Microscopic Particle Count Test). Using light obscuration, the FDA requirement for subvisible particulates in parenteral drug product is ≤6,000 particles per container for particles 10 micrometers in diameter, and ≤600 particles per container for particles ≥25 micrometers in diameter. Using the microscopic method, the FDA requirement for subvisible particulates in parenteral drug product is ≤3,000 particles per container for particles ≥10 micrometers in diameter, and ≤300 particles per container for particles 25 micrometers in diameter. Presently, no specification exists for particles of less than 10 micrometers in diameter, but the FDA has requested that particles of 2 to 10 micrometers be measured.

Particles of greater than 1 micrometer in diameter were measured using HIAC light obscuration and Brightwell micro-flow imaging (MFI). HIAC combines light obscuration with laser light scattering enabling the detection and counting of particles ranging from 500 nm-350 m in a moving fluid stream. Particles were sized based on voltage response generated in the detector and sorted into pre-determined size ranges based on voltage response.

For HIAC assays, samples from a manufacturing line (GMP lots) containing a monoclonal antibody at 150 mg/mL were pooled to a total volume of 25 mL. For each pooled sample, three readings of five milliliters per sample were made. Laboratory samples of the same 150 mg/mL antibody formulation were also examined by HIAC. Samples from at least three vials (2.5 mL/vial), seven 1-mL syringes (1.14 mL/syringe), or five 2.25-mL syringes (2 mL/syringe) were pooled, and three reading of one milliliter per reading were made. HIAC 9703 and HIAC 8000A instruments (Hach Company, Loveland, Colo.) using the HRLD 400 probe (which reads up to 18,000 cumulative counts per mL) and MC05 probe (which reads up to 10,000 cumulative counts per mL) respectively, were used to make the light obscuration readings.

The MFI method used less material (i.e., 1 mL of formulation, or 1 stability vial or syringe) than HIAC light obscuration and yielded higher particulate numbers than HIAC. Since MFI is microscopy-based, that method was more sensitive to the translucent protein particulates and was able to differentiate silicone oil droplets/air bubbles from protein particulates for prefilled syringe samples. MFI was conducted on a laboratory sample containing 150 mg/mL of a monoclonal antibody (as in the HIAC analyses). For MFI, one reading of one milliliter per reading was made.

### Determination of Polysorbate Degradation

Degradation of polysorbate was examined using one or more of several methods. The first method employed an enzymatic colorimetric assay to quantify non-esterified fatty acids (NEFA). The NEFA-HR(2) kit (Wako Diagnostics, Richmond, Va.) was used to detect fatty acids in formulated drug substance containing polysorbate. Briefly, the samples were combined with ATP and coenzyme A (CoA) in the presence of acyl-CoA synthetase (ACS). Available (free) fatty acids reacted with the CoA to form acyl-CoA. The acyl-CoA product was reacted with oxygen and acyl-CoA oxidase to produce trans-2,3-dehydroacyl-CoA and hydrogen peroxide. Peroxidase catalyzed the reaction of the hydrogen peroxide with 4-aminoantipyrine and 3-methyl-N-ethyl-N−(β-hydroxyethyl)-aniline to form a blue purple pigment (maximum absorbance at 550 nm). The amount of NEFA in the sample is proportional to the amount of pigment. For a detailed description of the NEFA colorimetric assay, see Duncombe, “The Colorimetric Micro-Determination of Non-Esterified Fatty Acids in Plasma,” Clin Chim Acta. 9:122-5 (1964); Itaya and Ui, “Colorimetric Determination of Free Fatty Acids in Biological Fluids,” J. Lipid Res. 6:16-20 (1965); Novak, M., “Colorimetric Ultramicro Method for the Determination of Free Fatty Acids,” J. Lipid Res. 6:431-3 (1965); and Elphick, M. C., “Modified Colorimetric Ultramicro Method for Estimating NEFA in Serum,” J. Clin. Pathol. 21(5):567-70 (1968).

The test sample containing the protein of interest (and putative host cell protein contaminant) was applied to a 10 kDa molecular weight cut-off filter. The retentate was recovered in 10 mM histidine (pH 6.0) at greater than 100 g/L protein and spiked with polysorbate to give a test sample of 100 g/L protein, 0.8% (w/v) polysorbate, 10 mM histidine, pH 6.0 (tᵢₙᵢₜᵢₐₗ). The test sample was subjected to 45° C. for 44 hours (t_{final}). Some samples were spiked with oleic acid to evaluate the recovery efficiency of NEFA in the samples. Percent polysorbate degradation was calculated as follows:

$$\frac{( [ NEFA ] {t}_{final} - [ NEFA ] {t}_{initial} )}{[ polysorbate ] - [ NEFA ] {t}_{final}} \times 100 \%$$

The second method for determining polysorbate degradation was based on mass spectroscopy. Using LC/MS analysis, this assay allowed the measurement and comparison of the initial percentage of esters and remaining percentage of esters in polysorbates after incubation at 45° C. at different time points. MAb1 produced according to process 6 (without HIC and with PS degradation activity) and mAb1 produced according to process 3 (with HIC step and without PS degradation activity) (see Example 8 and Table 9) were included as negative and positive controls, respectively.

Briefly, 15 mg of antibody sample (on the order of 5-10 mg/mL, or 7 mg/mL±1.5 mg/mL) was applied to an ultra-filter (Amicon Ultra 50K, Millipore, Billerica, Mass.) and centrifuged at 14,000×g for 15 minutes or until the remaining volume was slightly below the 100 μL marking on the device. 1 L of 10% polysorbate was added into the spin filter with the concentrated protein followed with vortexing. The sample was recovered by inverted centrifugation for 5 minutes at 1000 g to recover the full volume in the collection tube.

The recovered volume was measured and the concentration of polysorbate calculated. 1 μL of each recovered sample was and diluted 100-fold in a separate tube, and the protein concentration measured with Nanodrop 1000 (Thermo Fisher Scientific, Inc., Wilmington, Del.). The samples were then diluted in histidine buffer (10 mM, pH 6.0) and polysorbate stock to achieve 150 mg/mL protein concentration and 0.2% (w/w) polysorbate concentration.

Time zero (TO) sample (2 μL) was reserved from each sample and stored at −80° C. until used. Samples to be tested were sealed under argon and incubated at 45° C. to induce degradation, and removed for testing at the prescribed time points. 2 μL was taken from each of the samples at each time point and diluted with water to 100 μL. Each diluted time point sample was stored at −80° C. storage. After collection of each time point, the head space of the sample tube was filled with argon gas, the sample container resealed, and the sample returned to the incubator to resume incubation.

The time point samples were analyzed using an anion exchange column (Oasis MAX column, 30 μm, 2.1 mm×20 mm; Waters Corporation, Milford, Mass.) followed at t=5 minutes with reverse phase chromatography (ACQUITY UPLC® BEH 130 C4 column, 1.7 μm, 2.1 mm×50 mm; Waters Corporation, Milford, Mass.). The reverse phase output was connected to a mass spectrometer (Thermo Q-Exactive mass spectrometer; Thermo Fisher Scientific, Inc., Wilmington, Del.). The chromatographic conditions are described in Table 2.

The system was equilibrated with 99% mobile phase A (0.1% formic acid in water) at a flow rate of 0.1 mL/minute for 40 minutes prior to first injection. Water was used as a blank injection. The mass spectrometer parameters were as follows: mass range 150-2000 m z; heater temperature at 250° C.; voltage 3.8 kv; sheath gas 40; auxiliary gas 10; capillary temperature 350° C.; and S-lens 50. When mass spectrometry-based identification was not necessary, charged aerosol detection (CAD) was used an analytical flow rate and a desolvation temperature at 100° C. (Lisa et al., “Quantitation of triacylglycerols from plant oils using charged aerosol detection with gradient compensation,” J Chromatogr A. 1176(1-2):135-42 (2007); Plante et al., “The use of charged aerosol detection with HPLC for the measurement of lipids,” Methods Mol Biol. 579:469-82 (2009)).

TABLE 2
Chromatography conditions for determination of polysorbate degradation

| UPLC System | Waters ACQUITY UPLC I-Class/Dionex UltiMate 3000 | | | | |
|---|---|---|---|---|---|
| Mobile Phase | A: 0.1% formic acid in water | | | | |
| | B: 0.1% formic acid in acetonitrile | | | | |
| Column | Waters Oasis ® MAC 30 μm, 2.1 × 20 mm, | | | | |
| | Part No. 186002052 ACQUITY UPLC ® BEH130 | | | | |
| | C4 column, 1.7 μm, 2.1 mm × 50 mm from Waters, Part No. 186004496 | | | | |
| Column | 40° C. ± 1° C. | | | | |
| Temperature | | | | | |
| Autosampler | 5° C. ± 2° C. | | | | |
| Temperature | | | | | |
| Injection Volume | 20.0 μL | | | | |
| | Time (minute) | % A | % B | Flow (μL/minute) | Curve |
| Gradient | Initial | 99.0 | 1.0 | 100 | Initial |
| | 1.0 | 99.0 | 1.0 | 100 | Linear |
| | 5.0 | 85.0 | 15.0 | 100 | Linear |
| | 40.0 | 1.0 | 99.0 | 100 | Linear |
| | 45.0 | 1.0 | 99.0 | 100 | Linear |
| | 45.1 | 99.0 | 1.0 | 100 | Linear |
| | 50.0 | 99.0 | 1.0 | 100 | Linear |

To estimate the total amount of polyoxyethylene (POE), the mass chromatogram was extracted using the 300-800 m z range to avoid interference from degraded proteins, and the cluster of peaks from about 8-15 minutes was integrated. For CAD chromatograms, the first cluster of POE peaks was directly integrated from about 8-15 minutes (again, retention time may shift slightly). When there were other species co-eluting with the POE, the baseline was adjusted to minimize their impact on the peak area.

To estimate the total amount of POE esters, the mass chromatogram was extracted using the 300-2000 m z range, and the cluster of peaks from about 17-40 minutes was integrated. For the CAD chromatograms, the POE esters peak cluster was directly integrated from about 17-40 minutes.

Percentage of POE esters was calculated according to the following equation:

$$\frac{POE esters peak area}{POE esters peak area + POE area} \times 100 \%$$

Percentage of remaining POE esters was calculated according to the following equation:

$$\frac{\% POE esters at tn}{\% POE ester at t 0}$$

wherein n=2, 4, or 10 days.

### Example 5. Failure of Particulate Specification

Two GMP lots of a 150 mg/mL antibody formulation were assessed for subvisible particles via HIAC light obscuration after at least six months storage at 5° C., as described in U.S. Pat. No. 10,342,876, which is herein incorporated by reference. The formulation comprised 0.02% polysorbate 20 from supplier A, and 150 mg/mL anti-IL-4R antibody. The antibody was purified from CHO cell culture using a combination of affinity capture and ion exchange chromatography. The results are presented in Table 3.

TABLE 3
Number of particulates ≥ 10 μm in size after storage

| | | | | | 25° C. |
|---|---|---|---|---|---|
| | 5° C. Storage | | | | Storage |
| Lot | 0 months | 6 months | 9 months | 12 months | 6 months |
| 1 | 125 | 6,299 | 13,361 | 29,505 | 32,744 |
| 2 | 18 | 353 | 8,027 | 10,797 | 18,602 |

### Example 6. Quality and Purity of Fatty Acid Ester Affects SVP Formation

The effect of the nature and quality of the non-ionic detergent (polysorbate 20 and polysorbate 80) on subvisible particle formation in a protein formulation was tested by formulating an antibody in either (i) polysorbate 20 from supplier A (PS20-A), (ii) polysorbate 20 from suppler B (PS20-B), or (iii) polysorbate 80 (PS80), as described in U.S. Pat. No. 10,342,876, which is herein incorporated by reference. Table 4 shows HIAC SVP (≥10 μm SVPs) data from the formulated drug substance of the following formula: 20 mM histidine (pH 5.9), 12.5 mM acetate, 0.02% non-ionic detergent (polysorbate), 5% sucrose (w/v), 25 mM arginine, and 150 mg/mL antibody, stored as 2.5 mL fill in a 5 mL Type 1 borosilicate glass vial with a West S2-F451 4432/50 GRY B2-40 stopper.

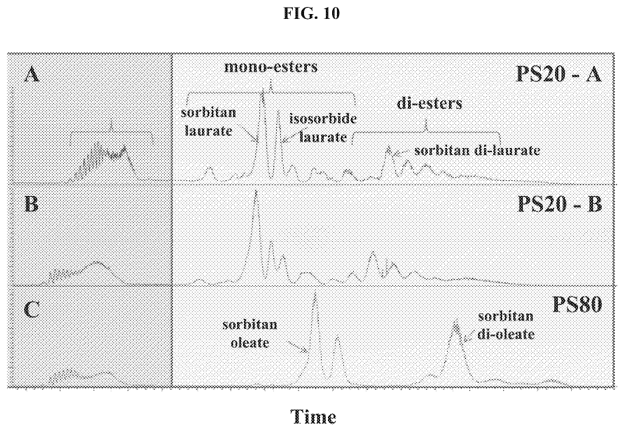

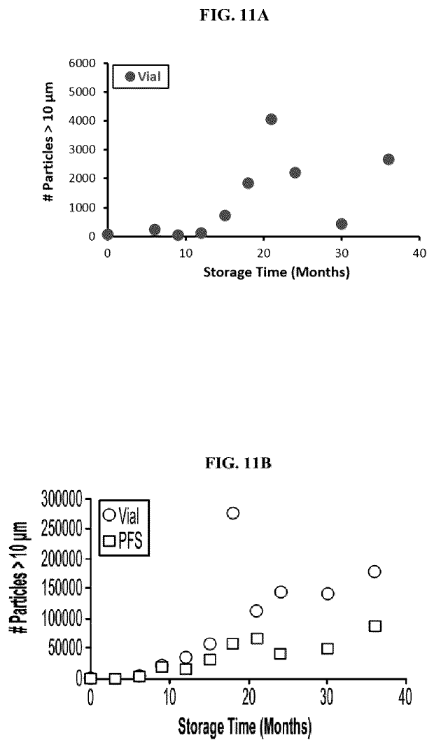

Here, formulated drug substance (“mAb1”) containing polysorbate 80 showed significantly less SVP formation over time than those formulations containing polysorbate 20. Furthermore, formulations containing polysorbate 20 from supplier B (PS20-B), which is a higher grade of polysorbate 20, showed less SVP formation than those formulations containing polysorbate 20 from supplier A (PS20-A; a lower grade of polysorbate 20). A comparative analysis of PS20-A and PS20-B shows that PS20-B has 5-10% more overall esters than PS20-A, and that PS20-A has more isosorbide laurate ester than does PS20-B, as shown in FIG. 10.

TABLE 4
Number of particulates ≥10 μm in size after storage with varying detergents

| | | 5° C. Storage | | | | 25° C. Storage |
|---|---|---|---|---|---|---|
| Lot | Non-ionic detergent | 0 months | 6 months | 9 months | 12 months | 6 months |
| 1 | PS20-A | 125 | 6,299 | 13,361 | 29,505 | 32,744 |
| 2 | PS20-A | 18 | 353 | 8,027 | 10,797 | 18,602 |
| 1 | PS20-B | 25 | 175 | NA | 1,138 | 4,221 |
| 2 | PS20-B | 8 | 108 | NA | 1,198 | 5,682 |
| 1 | PS80 | 19 | 26 | NA | 22 | 92 |

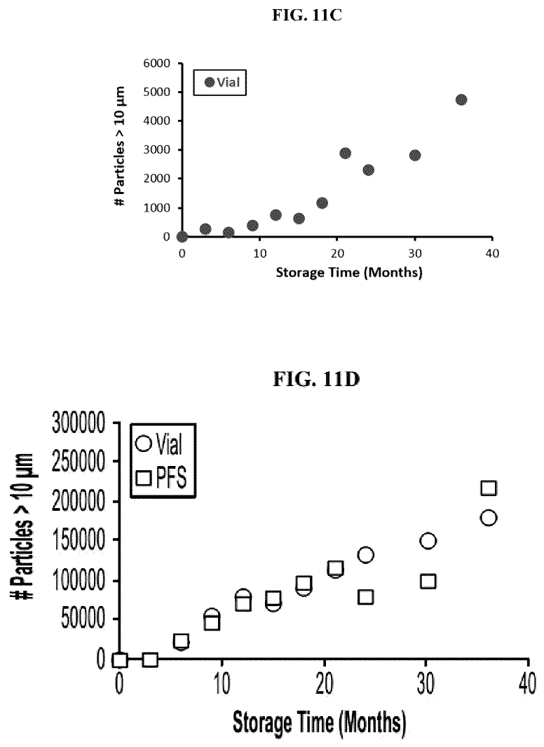

Formulated drug substance comprising HP-PS20 or SR-PS80 was further assessed using a membrane microscopy method or MFI after storage for up to 36 months, as shown in FIG. 11. Number of particles >10 μm was additionally compared between formulations stored in a glass vial compared to formulations stored in a pre-filled syringe, as shown in FIGS. 11A, 111B, 11C and 11D. In all cases, the number of particles increased substantially over time.

It was hypothesized that degradation of the fatty acid ester of polysorbate in the formulations may promote protein instability and consequently result in SVP formation. In order to assess polysorbate degradation, the stability of polysorbate 20 and polysorbate 80 in the 150 mg/mL antibody (mAb1) formulation containing 0.02% non-ionic detergent (polysorbate) prepared without HIC (process 3, see below and Table 9)) were compared. The relative amounts of remaining esters (mono- and di-esters) were determined by mass spectroscopy. Significant degradation of the ester components of polysorbate 20 was observed after the samples were stored at 5° C. for six months or 45° C. for two months. Less extensive degradation was observed for polysorbate 80 under the same conditions (see Table 5). These results correlate with the SVP particle formation observations.

The rates of degradation of polysorbate 20 and polysorbate 80 formulated with 150 mg/mL antibody (mAb1) (as described above for Table 5) were determined under identical conditions using mass spectroscopy to measure relative amounts of free fatty acids and fatty acid esters. Percent ester degradation was determined using the following formula:

$$\frac{\% POE esters at T 0 - \% POE esters at T 1}{\% POE esters at T 0}$$

wherein T0=time zero, T1=time at experimental condition (i.e., 2 months at 45° C.; 6 months at 5° C.), and POE=polyoxyethylene. Table 6 shows percent degradation of polysorbate 20 and polysorbate 80 in 150 mg/mL antibody formulations. The degradation rate of polysorbate 80 was consistently lower for mAb1 (but not for all antibodies tested) than the degradation rate of polysorbate 20 in otherwise identical antibody formulations.

TABLE 5
Percent remaining esters after storage

| | | | 6 months at | 2 months at |
|---|---|---|---|---|
| Detergent/chemical entity | | 0 months | 5° C. | 45° C. |
| PS20 | Monoester | 100% | 60% | 30% |
| | Diester | 100% | 30% | 10% |
| PS80 | Monoester | 100% | 80% | 35% |
| | Diester | 100% | 75% | 25% |

TABLE 6
Percent ester degradation after storage

| | | 6 months at | 2 months at |
|---|---|---|---|
| Polyoxyethylene ester | 0 months | 5° C. | 45° C. |
| PS20 | 0% | 22% | 63% |
| PS80 | 0% | 13% | 47% |

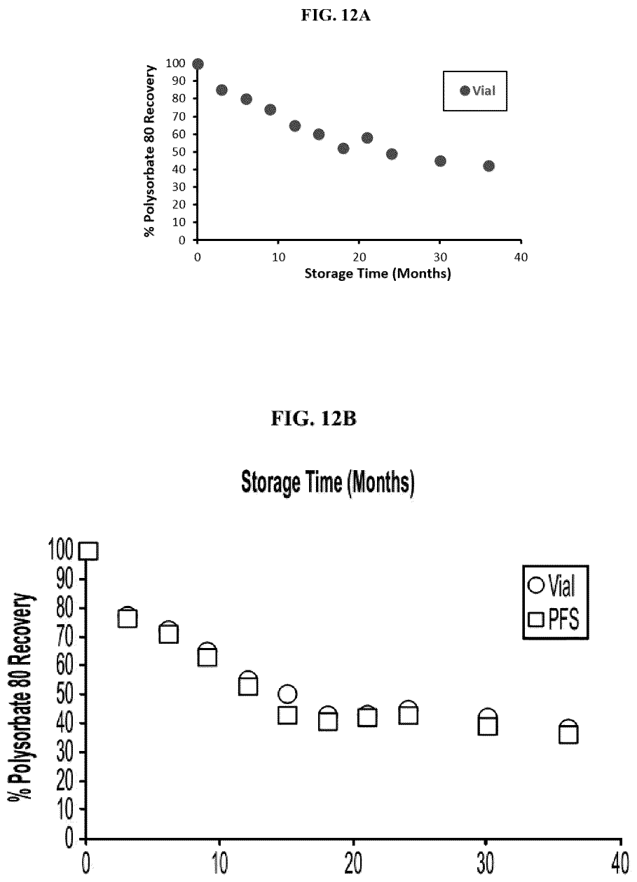

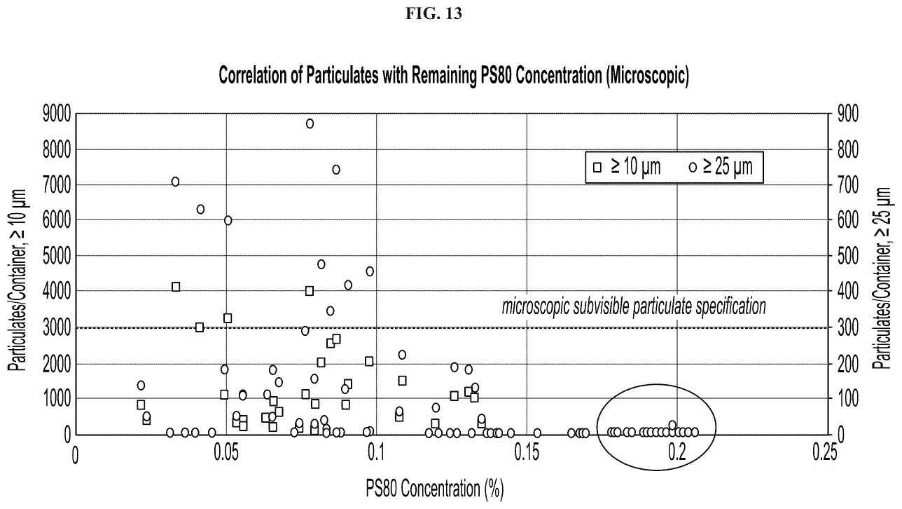

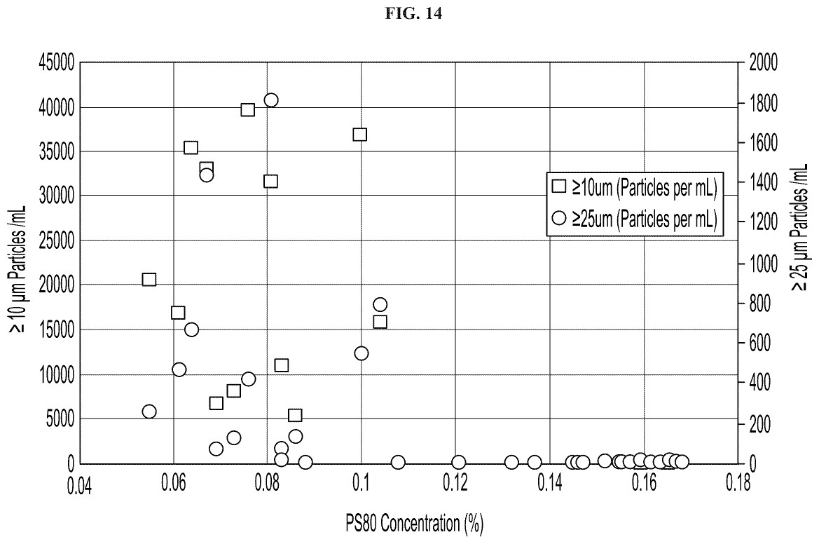

Polysorbate degradation in formulations comprising SR-PS80 or HP-PS20, stored in glass vials or pre-filled syringes, was further compared using CAD-UHPLC, as shown in FIGS. 12A and 12B. In all cases, there was appreciable polysorbate degradation over time. The presence of SVPs correlated with remaining polysorbate concentration in a formulation, as shown in FIG. 13. The same relationship was found using a formulation comprising another monoclonal antibody, mAb5, as shown in FIG. 14. Using Raman spectroscopy, it was confirmed that particulates in polysorbate-containing formulations matched the characteristics of fatty acids. Therefore, in order to solve the problem of SVP formation, the phenomenon of polysorbate degradation and free fatty acid particle formation was further investigated.

### Example 7. Polysorbate Degradation Activity

To determine the etiological agent responsible for polysorbate degradation, the buffered mAb1 antibody (150 mg/mL) was separated into two fractions by 10 kDa filtration: a protein fraction, and a buffer fraction, as described in U.S. Pat. No. 10,342,876, which is herein incorporated by reference. These two fractions, as well as intact buffered antibody, were spiked with 0.2% (w/v) of super refined polysorbate 20 (PS20-B) and stressed at 45° C. for up to 14 days. The study showed that the protein fraction, not the buffer fraction, had an effect on the degradation of sorbitan laurate (i.e., the major component of polysorbate 20), as shown in Table 7, and that the degradation of polysorbate 20 was correlated with the concentration of the antibody, as shown in Table 8.

TABLE 7
Degradation of sorbitan laurate in each mAb1 fraction

| Fraction | Sorbitan laurate % ester remaining (14 days at 45° C.) |
|---|---|
| Drug substance | 75% |
| Protein fraction | 75% |
| Buffer fraction | 100% |

TABLE 8
Degradation of polysorbate in each mAb1 fraction
correlates to antibody concentration

| | Antibody concentration | Polysorbate 20% ester |
|---|---|---|
| Fraction | (mg/mL) | remaining (12 days at 45° C.) |
| Drug | 150 | 82% |
| substance | | |
| Protein | 75 | 92% |
| fraction | | |
| Buffer fraction | 25 | 98% |

### Example 8. Hydrophobic Interaction Chromatography

Antibody was produced in a CHO cell host and purified using one of two processes (see Table 9), as described in U.S. Pat. No. 10,342,876, which is herein incorporated by reference. In one case, the antibody was purified using ion exchangers as polishing steps (capture step, ion exchange 1, ion exchange 2; “Process 3”). In the other case, one of the polishing steps used to purify the antibody was hydrophobic interaction chromatography as an additional polishing step (capture step, ion exchange, hydrophobic interaction; “Process 6”). The antibody, purified by either process 3 or process 6, was formulated at 150 mg/mL in 20 mM histidine (pH 5.9), 12.5 mM acetate, 5% sucrose, 25 mM arginine, and 0.02% polysorbate 20, and subjected to forced degradation at 45° C. for up to 14 days. At day 14, about 98% of the sorbitan laurate (i.e., intact ester) remained in the formulation containing the antibody purified using process 6, whereas only about 28% of the sorbitan laurate remained in the formulation containing the antibody purified using process 3. Therefore, the hydrophobic interaction chromatography (HIC) step likely removed an activity contributing to polysorbate degradation.

TABLE 9
Polysorbate recovery using various antibody purification processes

| Process | | |
|---|---|---|
| no. | Purification steps | % intact PS20 |
| 1 | Protein A affinity capture (PA) | 54% |
| 2 | PA > cation exchange (CEX) | 25% |
| 3 | PA > CEX > anion exchange (AEX) | 86% |
| 4 | PA > CEX > hydrophobic interaction (HIC) | 90% |
| 5 | PA > AEX | 83% |
| 6 | PA > AEX > HIC | 92% |

The role of bulk process steps in removing the putative polysorbate degradation factor (putative esterase activity) was evaluated. Antibody produced from CHO cells was subjected to sequential purification steps, and the stability of polysorbate 20 was assessed at each step. The results from one set of experiments are presented in Table 9, which reports on the percent intact polysorbate 20 at each step or sequence of steps. Percent intact polysorbate 20 is predicted to be inversely proportional to the amount of contaminant esterase activity.

Multiple different antibodies were tested for an associated polysorbate degrading activity (esterase) and the effect of HIC on that activity. In each case, polysorbate 20 degradation activity was detected, and that activity was virtually ablated by the incorporation of a HIC purification step (Table 10).

TABLE 10
Polysorbate degradation with and without HIC purification

| Antibody | HIC or no HIC | PS20 degradation at day 15 |
|---|---|---|
| mAb1 (IgG4) | No HIC | 72% |
| mAb1 (IgG4) | HIC | 2.0% |
| mAb2 (IgG1) | No HIC | 41% |
| mAb2 (IgG1) | HIC | 2.0% |
| mAb3 (IgG4) | No HIC | 40% |
| mAb3 (IgG4) | HIC | 5.0% |
| mAb4 (IgG4) | No HIC | ND |
| mAb4 (IgG4) | HIC | 1.0% |

The role of HIC in subvisible particle formation was explored. Without meaning to be limited by theory, it was hypothesized that the stability of the non-ionic detergent in a protein (e.g., antibody) formulation is directly correlated to the formation of subvisible particles. Loss of surfactant activity may allow protein to aggregate and form subvisible particles. Additionally or alternatively, the fatty acids released by the degrading sorbitan fatty acid esters may also contribute to subvisible particle formation as immiscible fatty acid droplets. Therefore, levels of subvisible particles ≥10 micrometers in diameter were counted in drug substance (150 mg/mL antibody in 20 mM histidine (pH 5.9), 12.5 mM acetate, 5% sucrose, 25 mM arginine, and 0.02% polysorbate 20) produced with HIC (e.g., process 6) or without HIC (e.g., process 3). The results (presented in Table 11 and Table 12) show that the application of a HIC step significantly reduced the formation of SVPs in the drug substance (on the order of ten-fold less), even though the lower quality PS20-A was used in these experiments.

TABLE 11
Number of particles ≥10 μm in size
with and without HIC purification

| | | | | | 25° C. |
|---|---|---|---|---|---|
| | 5° C. Storage | | | | Storage |
| Process | 0 months | 6 months | 12 months | 24 months | 6 months |
| HIC used | 129 | 189 | 140 | 178 | 136 |
| No HIC | 35 | 769 | 1,342 | 14,346 | 1,951 |

TABLE 12
Number of particles ≥25 μm in size
with and without HIC purification

| | | | | | 25° C. |
|---|---|---|---|---|---|
| | 5° C. Storage | | | | Storage |
| Process | 0 months | 6 months | 12 months | 24 months | 6 months |
| HIC used | 15 | 12 | 28 | 28 | 6 |
| No HIC | 3 | 59 | 30 | 429 | 70 |

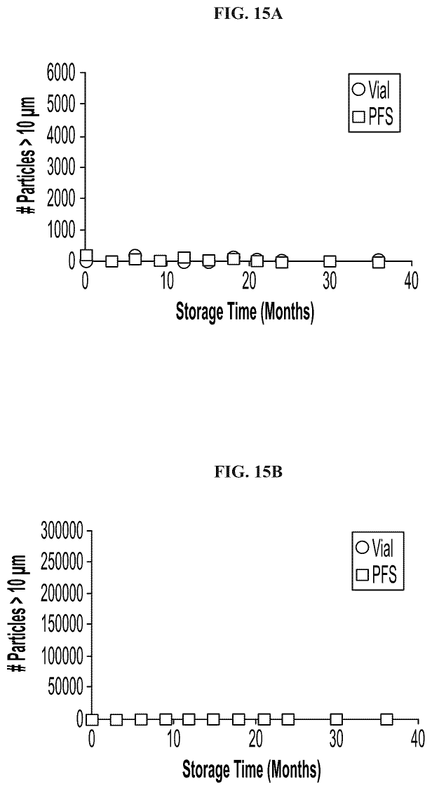

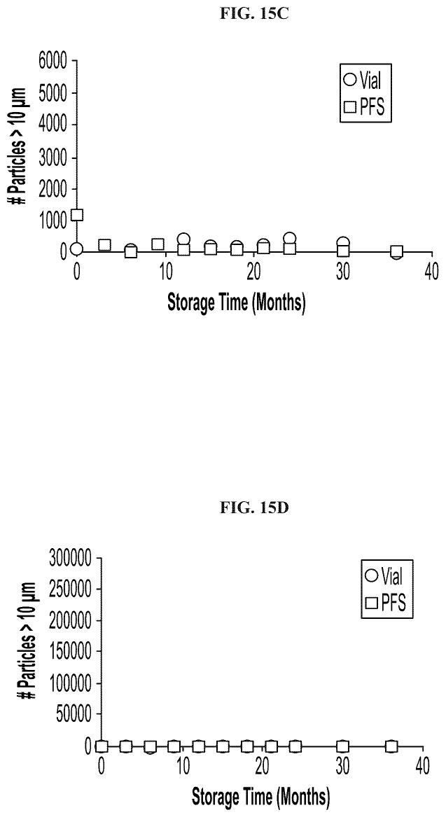

mAb1 formulations comprising PS20 or PS80 and produced with HIC were further characterized over time for the formation of particles, as shown in FIGS. 15A, 15B, 15C and 15D. Formulations comprising either PS20 or PS80, as measured by either membrane microscopy or MFI, did not show an increase in particle formation over time.

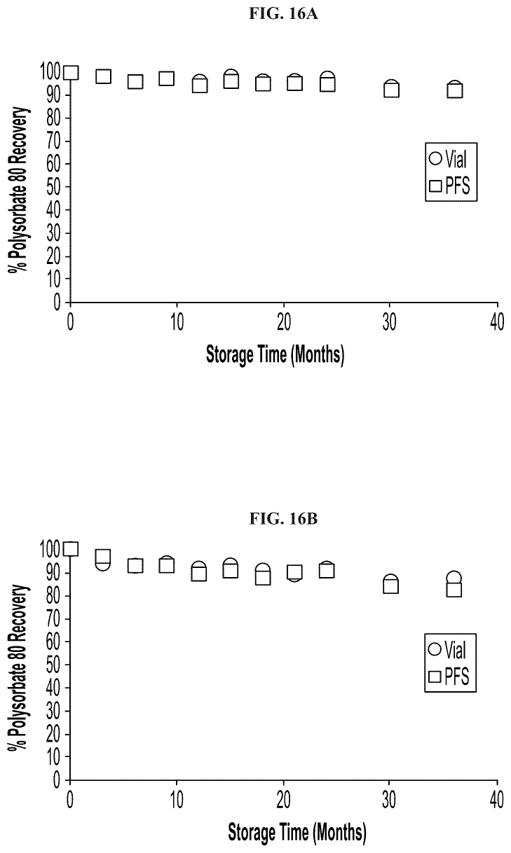

mAb1 formulations produced with HIC were further evaluated for polysorbate degradation over time, as shown in FIGS. 16A and 16B. In agreement with the SVP results described above, formulations subjected to the HIC process did not show appreciable polysorbate degradation even over 36 months of storage.

### Example 9. Putative Phospholipase B-Like 2 Activity

Polysorbate degradation activity was followed during HIC purification of an exemplary antibody produced in CHO cell culture, as described in U.S. Pat. No. 10,342,876, which is herein incorporated by reference. Partially purified CHO cell extract was applied to HIC (phenyl-sepharose). The flow-through, which contained almost all of the antibody, was collected and analyzed for polysorbate degradation activity. No polysorbate degradation activity was observed in this flow-through fraction. The HIC bound fraction was stripped from the HIC media and subsequently subjected to 100 kDa cut-off ultrafiltration/diafiltration. The unfiltered stripped fraction contained 9.9% polysorbate degradation activity, the filter permeate contained 1.3% polysorbate degradation activity and 5% antibody yield, and the filter retentate contained 7.4% polysorbate degradation activity and 95% antibody yield.

TABLE 13
Percent reduction in polysorbate 20 degradation
by concentration of lipase inhibitor

| Lipase inhibitor | 0 mM | 0.001 mM | 0.01 mM | 0.1 mM |
|---|---|---|---|---|
| Orlistat | 0% | 0% | 27.8% | 67% |
| Diethylumbelliferyl | 0% | 48% | 100% | >95% |
| phosphate | | | | |
| URB602 | 0% | 0% | 20% | 0% |
| 2-Butoxyphenyl boronic | 0% | 0% | 28% | 0% |
| acid | | | | |

Whether the polysorbate degrading activity is a lipase was tested by combining a lipase inhibitor with the polysorbate degrading activity fraction spiked with polysorbate 20. Table 13 presents the data showing a reduction of polysorbate degrading activity due to lipase inhibitor relative to the control (antibody with associated polysorbate degrading activity plus polysorbate 20 without lipase inhibitor). Lipase inhibitors reduced or eliminated the polysorbate degradation activity associated with the antibody.

A CHO-produced recombinant antibody HIC strip fraction (not the flow-through), which contained the polysorbate degradation activity, was subjected to additional HIC in bind/elution mode, wherein the antibody was eluted with a shallow gradient. Elution fractions were tested for PS20 degradation activity and those fractions having that activity were subjected to (i) intact mass spectrometry analysis, (ii) native size exclusion chromatography UV analysis (SEC-UV), and (iii) tryptic digestion followed with LC-MS and proteomic search analysis. Intact mass spectrometry analysis of reverse phase liquid chromatography fractions revealed an unknown species in hydrophobic fraction L8 (the most hydrophobic fraction). Formulated antibody samples containing polysorbate 20 and spiked with L8 (1:100) showed 20% polysorbate degradation by day eight. Antibody monomer and free light chain were detected in less hydrophobic fractions L3-L7, as well as L8. Antibody dimer was detected in fractions L5-L8.

HIC strip fractions L3-L9 were subjected to SEC-UV under native conditions. Fraction L8 separated into three major peaks coming off first, and two minor peaks coming off later and representing smaller species. The first peak off the column contained antibody dimer and other oligomers. The second peak contained antibody monomer. The third peak contained the species having polysorbate degradation activity. Thus, the degradation activity is separable from the antibody and is of smaller molecular rotation than the antibody monomer.

HIC fraction L8 was also subjected to shotgun proteomics analysis. Briefly, the L8 fraction was sequentially (i) retained on a 10 kDa filter, (ii) reconstituted in 6M guanidine-HCl, 100 mM Tris-HCl, pH 7.5, (iii) treated for 30 minutes at 50° C. in 10 mM Tris(2-carboxythyl)phosphine hydrochloride) (TCEP) followed by 30 minutes in the dark at room temperature in 20 mM indole-3-acetic acid (IAA), (iv) diluted eight-fold, had trypsin added at 1 part trypsin to 20 parts sample, and incubated at 37° C. for four hours, and then (v) subjected to LC-MS/MS analysis. Proteomic searching of the resultant peptide sequences revealed five proteins associated with L8: (i) putative phospholipase B-like 2 (representing 15% of the peak fraction), (ii) peroxiredoxin-1, (iii) heat shock 27 kDa protein 1, (iv) anaphase-promoting complex subunit 1, and (v) U3 small ribonucleoprotein protein MPP10.

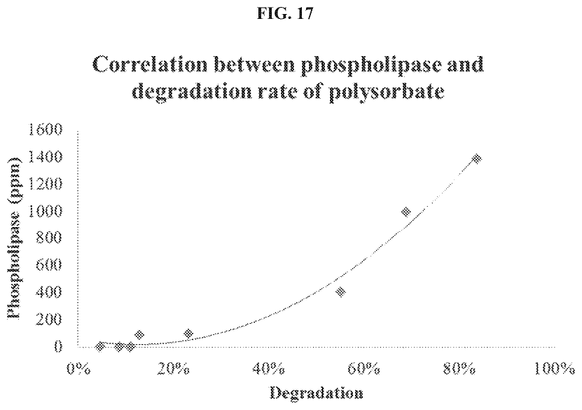

The amount of polysorbate degradation activity correlated with the abundance of phospholipase B-like 2 protein (PLBL2) present. At various purification steps, the amount of PLBL2 was determined via nanoLC-MS or LC-MS and the rate of polysorbate degradation (PS20 spiked fractions) was determined. The abundance of PLBL2 was calculated based on the ratio of peptide intensity from the lipase and drug substance (i.e., antibody). The results are presented in FIG. 17 and Table 14.

TABLE 14
Correlation of PLBL2 and polysorbate degradation

| | | Relative amount |
|---|---|---|
| Fraction | % PS20 degradation | PLBL2 (ppm)² |
| ProA pool | 69.02% | 991 |
| CEX pool | 55.24% | 403 |
| AEX pool | 12.85% | 84 |
| HIC pool | 8.67% | 0 |
| HIC pool 2 | 10.96% | 0 |
| HIC strip | 83.75% | 1384 |
| mAb1 process 3 (example 8) | 4.60% | 0 |
| mAb1 process 6 (example 8)¹ | 29.12% | 92 |

¹Degradation rate adjusted by concentration.
²Abundance of phopholipase calculated based on the ratio of peptide intensity from the lipase and drug substance.

### Example 10. Reduction of Lipase Activity Using AEX

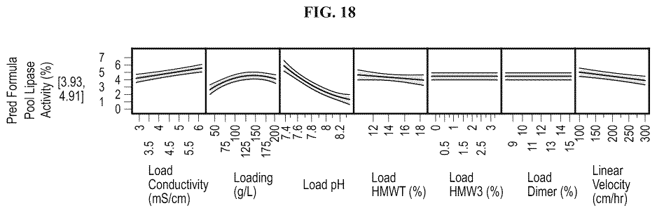

The ability of an anion exchange (AEX) chromatography unit operation to reduce levels of lipases in a drug formulation was investigated. AEX was performed in flow through mode, where negatively charged impurities are adsorbed to the immobilized, positively charged ligand (column), and the product flows through. Transfer functions were generated using stepwise regression for process parameters considered most likely to influence lipase activity. Visualizations of the resulting models are shown in FIG. 18. This model can be used for rational set point and range selection using an optimization algorithm to maximize desirability and robustness of the process. It was surprisingly discovered that lipase activity inversely correlated to AEX load pH, providing a new method for minimized lipase activity by optimizing AEX chromatography conditions.

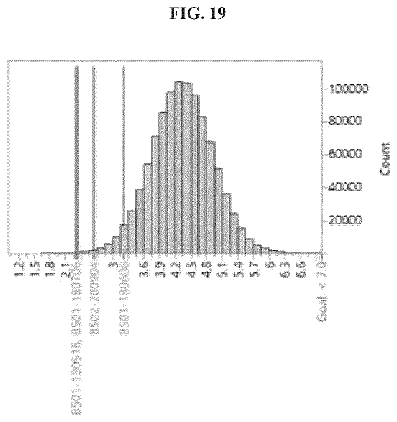

Performance of an optimized AEX process was verified in four pilot-scale (500 L) Confirmation Batches, as shown in Table 15. Lipase activity was measured by stressing concentrated samples at 45° C. for 44 hours after a 0.8% w/v polysorbate 20 spike and measuring change in non-esterified fatty acids (NEFA). Anion exchange process performance during these batches was compared to small-scale model predictions derived from Monte Carlo simulations of the process run at set point with estimated variation in input parameters, as shown in FIG. 19. Confirmation batch responses are indicated with a solid orange line. All responses were within the predicted range generated from the scale-down multivariate model for an anti-IL-4R antibody produced using the process of the present invention, illustrating process robustness to scale-up and appropriateness of the small-scale model to predict pilot scale performance.

TABLE 15
Summary of anion exchange step 500 L scale performance
during process confirmation batches

| | | | | | Confirmation | |
|---|---|---|---|---|---|---|
| | | | | | Batch (Mean ± | Scale-down |
| Bioreactor | B1 | B2 | B3 | B4 | SD, N = 4) | (Mean ± SD) |
| AEX Load Lipase | 6.31 | 6.30 | 6.29 | 7.28 | 6.55 ± 0.49 | N/A |
| Activity (%) | | | | | | |
| AEX Pool Lipase | 2.29 | 3.21 | 2.33 | 2.64 | 2.62 ± 0.42 | 4.3 ± 0.6 |
| Activity (%) | | | | | | |

SD, standard deviation

### Example 11. Optimized Fatty Acid Composition to Reduce Particle Formation

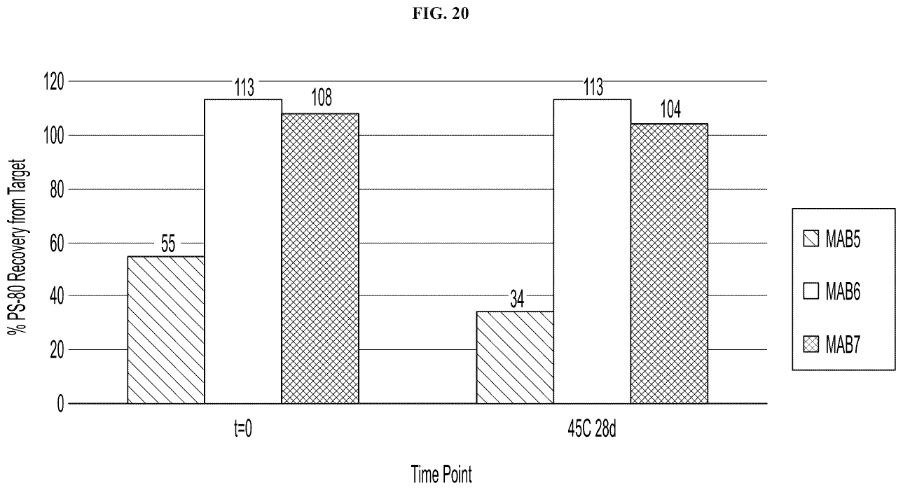

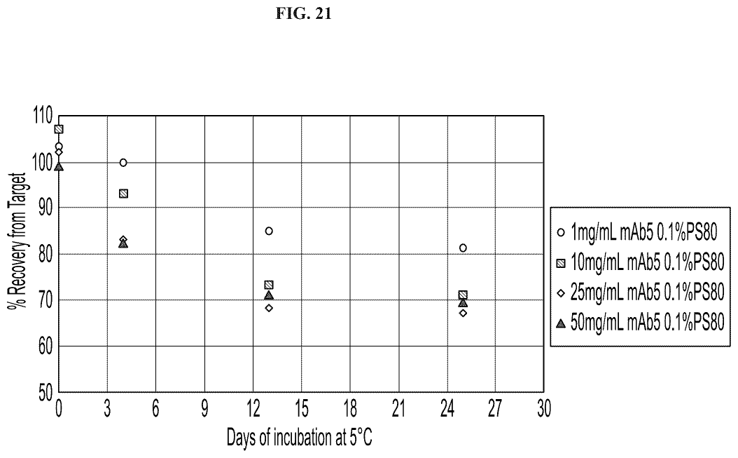

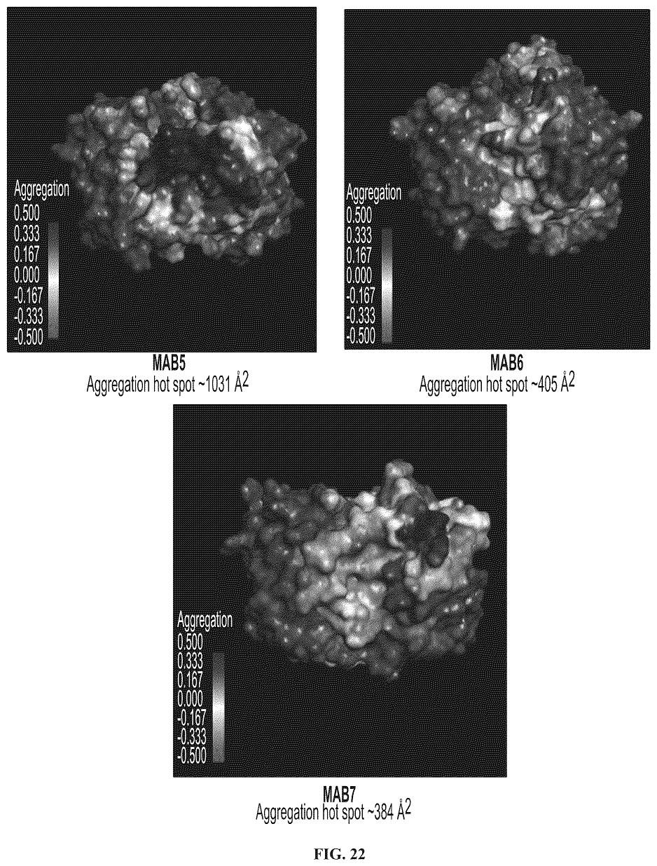

Proteins of interest may present additional challenges to lipase removal based on their structure and physicochemical characteristics. mAb5 was formulated following typical HCP removal techniques, but still showed polysorbate degradation over time in storage, as shown in FIG. 20 and FIG. 21. Homology modeling demonstrated that mAb5 features a large hydrophobic patch compared to mAb6 and mAb7, as shown in FIG. 22, which could cause co-elution of mAb5 with hydrophobic lipases. Therefore, further approaches to reducing polysorbate degradation and SVP formation were investigated.

Fatty acid composition and percent distribution of high melting point components are useful considerations for predicting particulate formation. Free fatty acids with high melting points can likely form insoluble particles that can be detected at room temperature and during analytical assessment. The composition of fatty acids in different polysorbates, along with their respective melting points, is shown in Table 16. Commercially available polysorbates may have fatty acid compositions with a wide range of melting points, with PS80 featuring a higher concentration of low melting point fatty acids, in particular oleic acid.

TABLE 16
Fatty acid compositions of commercially available polysorbates

| | | | | | | Croda High | Melting |
|---|---|---|---|---|---|---|---|
| Fatty | % (from | SR- | Seppic | Croda | NOF PS80 | Oleic SR- | Point |
| acids | CoA) | PS20 | PS80 | SR-PS80 | (ChP) | PS80 (ChP) | (° C.) |
| Capric | ≤10 | 1.7 | — | — | — | — | 31.6 |
| Lauric | 40.0-60.0 | 55.6 | — | — | — | — | 43.2 |
| Myristic | ≤5.0 | 22.8 | 0 | 0 | NMT0.1 | 0 | 54.4 |
| Palmitic | ≤16.0 | 10.8 | 12 | 1.4 | 0.2 | 0.1 | 62.9 |
| Stearic | ≤6.0 | 0.3 | 2.8 | 2.3 | NMT0.1 | 0 | 69.3 |
| Oleic | ≥58.0 (≥98.0 | 6.9 | 70.6 | 87.8 | 99.3 | 99.5 | 13.4/16.3 |
| | ChP) | | | | | | |

The storage stability of mAb1 drug product (DP) prepared without a HIC step was evaluated across samples comprising different grades of PS80, as described in U.S. Patent Application Publication No. 20190083618 A1, which is herein incorporated by reference. Each DP sample had a volume of 2.136 mL, contained the same concentration of mAb1 (150 mg/mL), and 0.2% (w/v) of one of several lots of PS80. Each PS80 lot had one of three different percentage contents of oleic acid ester (70%, 87%, and ≥99%). Table 17 summarizes the percentage content of oleic acid ester in the PS80 in each FDS sample.

TABLE 17

| Sample | % oleic acid ester content in PS80 (Lot) |
|---|---|
| DP A | 87% |
| DP B | ≥99% (Lot 1) |
| DP C | ≥99% (Lot 2) |
| DP D | 70% (Lot 1) |
| DP E | 70% (Lot 2) |
| DP F | 70% (Lot 3) |

The DP samples were stored at 2-8° C. in glass pre-filled syringes for up to 24 months. Particulates were measured in each DP sample every six months for a total of 24 months, by both membrane microscopy method and micro-flow imaging (MFI).

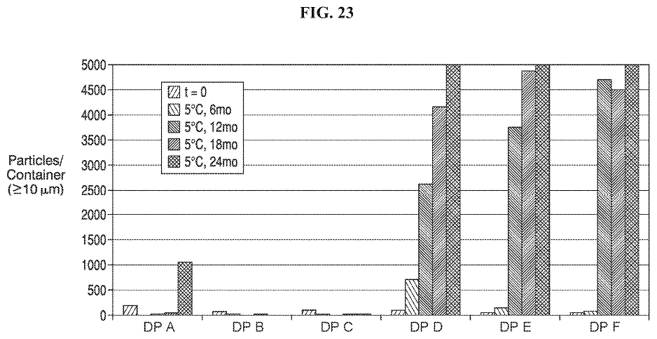

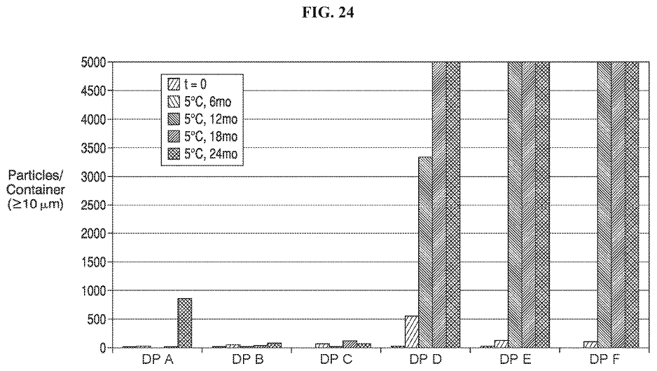

FIG. 23 shows the number of SVPs per container having a diameter of ≥10 μm, as measured by membrane microscopy. FIG. 24 shows, in chart form, the number of SVPs per container having a diameter of ≥10 μm, as measured by MFI. As shown in FIG. 23 and FIG. 24, DP B and DP C (the two DP samples containing PS80 having a ≥99% content of oleic acid esters) displayed the lowest numbers of SVPs across the full 24-month period, as measured by both membrane microscopy (FIG. 23) and MFI (FIG. 24). DP A, containing PS80 having an 87% content of oleic acid esters, displayed the next lowest number of subvisible particulates across the 24-month period (in particular, showing between 800 and 1200 particles by 24 months). DPs D, E, and F all showed well over 3000 particles per container (as measured by both methods) by at least the 18-month mark.

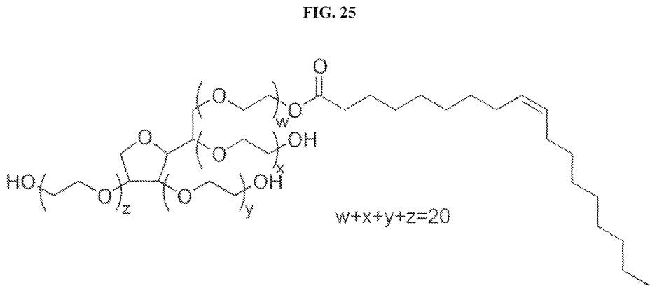

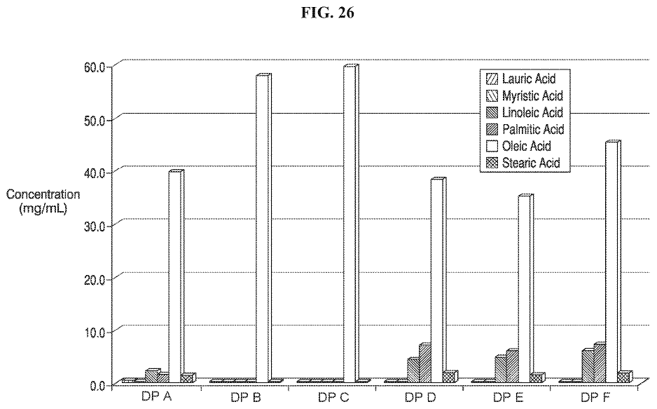

It was hypothesized that the lower numbers of particles in DPs A, B, and C (as compared to the more numerous particles in DPs D, E, and F) were a result of the use of PS80 having a higher percentage content of oleic acid (or long-chain fatty acid) esters. Oleic acid is a longer chain fatty acid, with one unsaturated bond (see FIG. 25). Therefore, it has a sub-ambient melting temperature of about 13° C. A precursor to subvisible and visible free fatty acid (FFA) particulate formation is the agglomeration of individual FFA chains into aggregates, which then precipitate in the form of particles. Oleic acid may be generated during storage of the formulations at 5° C. by, e.g., enzymatic hydrolysis of the fatty acid esters in polysorbate 80. This oleic acid may form SVPs, but due to its low melting temperature, such particles are more likely to exist as an oily liquid in protein formulations at room temperature (about 22° C.) where analysis is performed, and therefore does not persist as subvisible particulates at room temperature. As a contrast, higher amounts of non-oleic acid ester content in the formulation will lead to formation of their corresponding FFA upon hydrolysis, and due to their higher melting temperatures the subvisible and visible amorphous particulates thus formed persist at ambient temperature during analysis.

Additionally, oleic acid esters are better solubilizing/stabilizing agents than esters of shorter chain fatty acids due to oleic acid esters' higher hydrophobicity, which enables oleic acid esters to solubilize free fatty acid and protein particulates, thereby maintaining product stability. Therefore, polysorbate 80 with higher contents of oleic acid esters (>98%) can provide improved stability to protein formulations and drug products as compared to polysorbate 80 with lower contents of oleic acid esters.

A concentration of each type of free fatty acid (in micrograms/mL) in each sample DP (DP A-F) was evaluated after storage of the samples at 5° C. for 18 months. Sample DP A-F were prepared as described above. Free fatty acid concentrations were measured at 18 months by LC-MS, as shown in FIG. 26. DPs B and C (the two DP samples containing PS80 having a ≥99% content of oleic acid esters displayed the highest concentration of oleic acid, and the lowest concentrations of other FFAs. This indicates the homogeneity of the FFAs (i.e., oleic acids) in DPs B and C. This further indicates that the use of polysorbate comprising a high oleic acid concentration can reduce the formation of free fatty acid particles at ambient temperature even in a formulation comprising a lipase that causes free fatty acid production.

### Example 12. Prevention of Particle Formation in Lipase-Containing Formulations with PEG3350 or Poloxamer 188

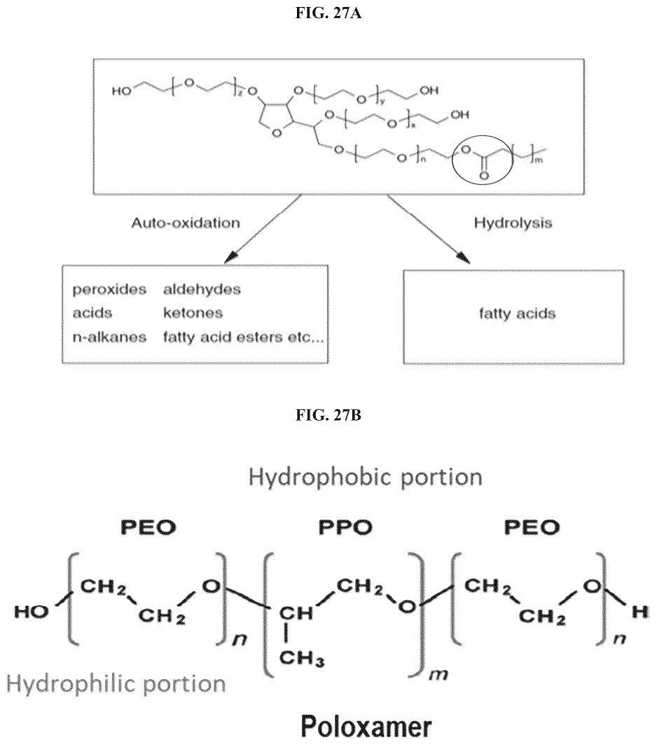

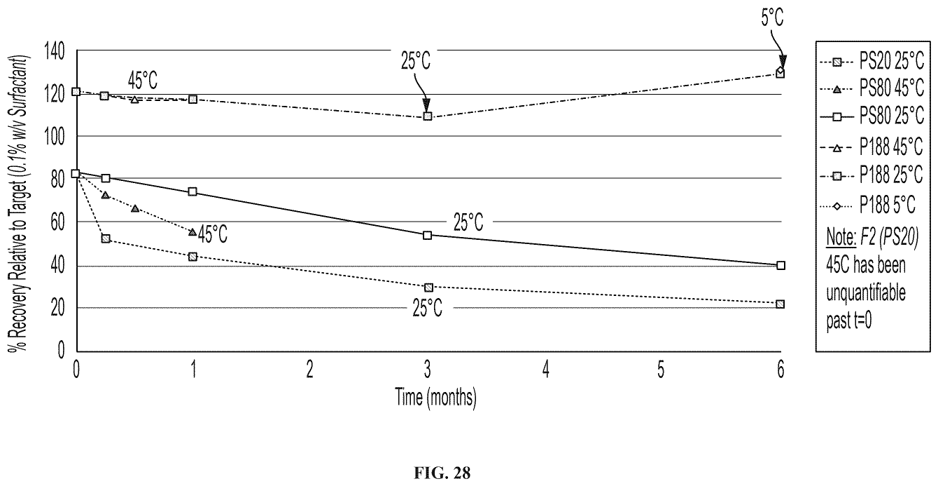

Esterases or lipases can hydrolyze polysorbates by enzymatic hydrolysis of the ester bond, as shown in FIG. 27A. Alternative surfactants such as PEG3350 and poloxamer 188 don't contain ester bonds and therefore are not targets for esterases, as shown in FIG. 27B. mAb5 was formulated with PS20, PS80, or poloxamer 188, and the recovery of surfactant was compared, as shown in FIG. 28. Unlike the PS20 and PS80 formulations, poloxamer formulations did not experience recovery losses, at all temperatures tested.

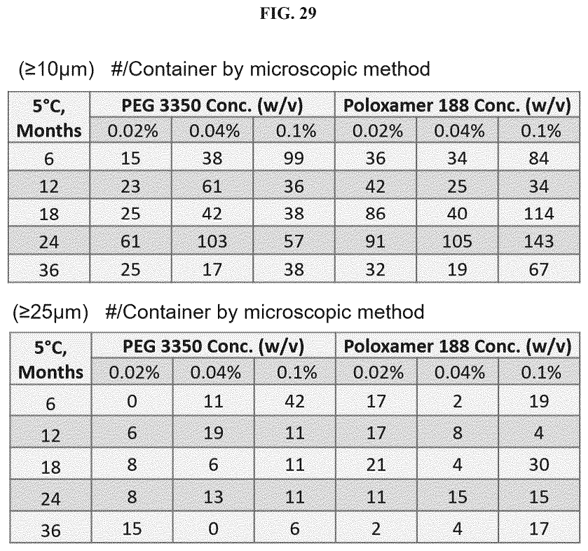

In order to investigate whether the use of alternative surfactants could reduce the formation of SVPs in a formulated DP, formulations of IL-4R antibody were prepared using alternative surfactants, and particulate formation was measured over time, as described in U.S. Provisional Application No. 63/337,532, which is herein incorporated by reference. An anti-IL-4R antibody comprising the HCVR/LCVR amino acid sequence pair of SEQ ID NOs:1/2 at a concentration of 150 mg/mL was formulated with 20 mM histidine, 12.5 mM sodium acetate, 25 mM arginine-HCl, 5% w/v sucrose, and either PEG3350 or poloxamer 188 at varying concentrations, at pH 5.9. The formulations were stored in syringes at 5° C. for up to 36 months with periodic measurements taken of the number of particles (≥10 μm and ≥25 μm) present in the formulations, as determined by microscopy. As shown in FIG. 29, few particles of ≥10 μm or ≥25 μm were identified in the formulations over the 36 month observation period. Moreover, no appreciable differences or changes in the number of subvisible particles among the different PEG3350- or poloxamer 188-containing formulations were observed over the course of the storage period.

SEQ ID NO: 1
EVQLVESGGGLEQPGGSLRLSCAGSGFTFRDYAMTWVRQAPGKGLEWVS
SISGSGGNTYYADSVKGRFTISRDNSKNTLYLQMNSLRAEDTAVYYCAK
DRLSITIRPRYYGLDVWGQGTTVTVS
SEQ ID NO: 2
DIVMTQSPLSLPVTPGEPASISCRSSQSLLYSIGYNYLDWYLQKSGQSP
QLLIYLGSNRASGVPDRFSGSGSGTDFTLKISRVEAEDVGFYYCMQALQ
TPYTFGQGTKLEIK

These results demonstrate that alternative surfactants such as poloxamer 188 are resistant to degradation in a lipase-containing formulation, and that low concentrations of PEG3350 or poloxamer 188 can prevent particle formation in a DP formulation over long storage periods.

In order to assess the capacity of alternative surfactants to promote therapeutic protein stability, formulations comprising PEG3350 or poloxamer 188 were further subjected to assessments of agitation stress stability and thermal stress stability. The agitation stress stability was tested for IL-4R antibody formulations containing different concentrations of the surfactant PEG3350 or poloxamer 188. An anti-IL-4R antibody comprising the HCVR/LCVR amino acid sequence pair of SEQ ID NOs:1/2 at a concentration of 150 mg/mL was formulated with 20 mM histidine, 12.5 mM sodium acetate, 25 mM arginine-HCl, 5% w/v sucrose, and PEG3350 or poloxamer 188 at varying concentrations, at pH 5.9. The formulations were stored in glass vials and agitated by vortexing (speed setting=4) for 30 minutes, 60 minutes, or 120 minutes. The percentage of high molecular weight (HMW) species was then determined by size exclusion ultra high performance liquid chromatography (SE-UPLC).

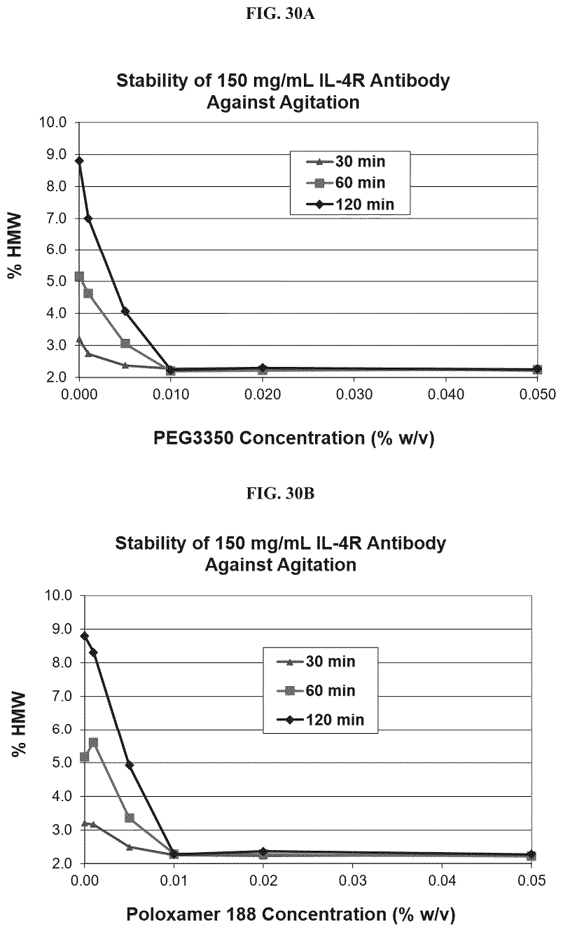

As shown in FIG. 30A, formulation of the antibody with at least 0.01% (w/v) PEG3350 prevented an observable increase of HMW species (quantitated by SE-UHPLC) due to the agitation. Lower amounts of PEG3350 (0.001% or 0.005%) were insufficient to prevent formation of HMW. Similarly, as shown in FIG. 30B, formulation of the antibody with at least 0.01% (w/v) poloxamer 188 prevented an observable increase of HMW species, but HMW species were observed with lower amounts of poloxamer 188 (0.001% or 0.005%).

The thermal stress stability of IL-4R antibody formulations containing PEG3350 or poloxamer 188 was additionally tested and compared to various IL-4R antibody formulations containing polysorbate. An anti-IL-4R antibody comprising the HCVR/LCVR amino acid sequence pair of SEQ ID NOs:1/2 at a concentration of 150 mg/mL was formulated with 20 mM histidine, 12.5 mM sodium acetate, 25 mM arginine-HCl, 5% w/v sucrose, and polysorbate 20, polysorbate 80, PEG3350 or poloxamer 188 at varying concentrations, at pH 5.9. These formulations were subjected to thermal stress (45° C.) for a period of up to 56 days, and the percentage of high molecular weight (HMW) species was determined by size exclusion ultra high performance liquid chromatography (SE-UPLC) at 7 days, at 14 days, at 28 days, at 42 days, and at 56 days.

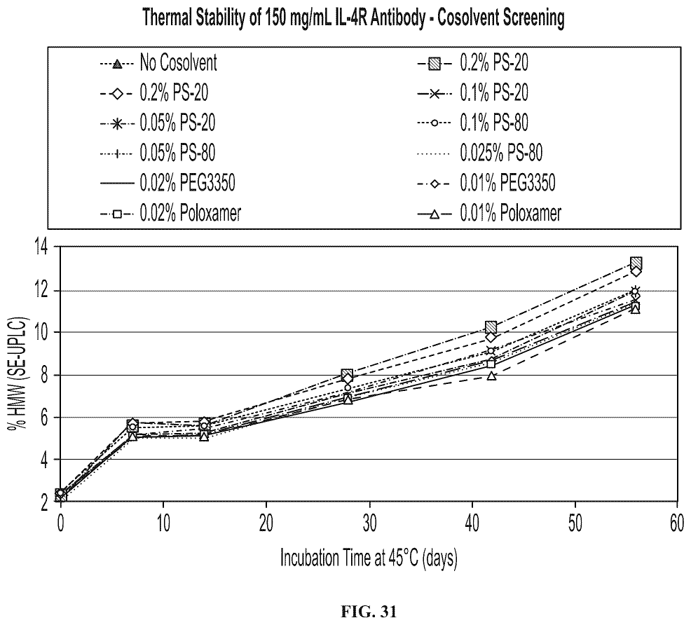

As shown in FIG. 31, antibody formulations comprising PEG3350 or poloxamer 188 at a concentration of 0.01% or 0.02% showed similar thermal stability to antibody formulations containing lower amounts of polysorbate (up to 0.1% w/v). By 28 days, the PEG3350- or poloxamer 188-containing formulations exhibited a lower percentage of HMW species than control formulations comprising 0.2% polysorbate 20.

Additional studies were carried out comparing the thermal stability of antibody formulations containing 0.2% polysorbate 80 (Table 18), 0.01% poloxamer 188 (Table 19), 0.1% poloxamer 188 (Table 20), 0.5% poloxamer 188 (Table 21), 0.01% PEG3350 (Table 22), 0.1% PEG3350 (Table 23), or 0.5% PEG3350 (Table 24). Each formulation comprised 150 mg/mL anti-IL4R antibody, 25 mM L-arginine-HCl, 20 mM L-histidine, 12.5 mM sodium acetate, 5% (w/v) sucrose, and the surfactant, at a pH of 5.9. Formulations comprising poloxamer 188 or PEG3350 showed stability benefits compared to a formulation comprising polysorbate 80.

TABLE 18
Thermal stability of formulations with 0.2% PS80

| | | | Length of Storage at 45° C. (months) | | |
|---|---|---|---|---|---|
| Assay | | t = 0 | 1 | 2 | 3 |
| Visual Appearance | | Pass | Pass | Pass | Pass |
| Increase in Turbidity (OD 405 nm) | | — | 0.02 | 0.16 | 0.33 |
| pH | | 6.0 | 6.0 | 6.0 | 6.0 |
| % antibody Recovered by RP-UPLC | | 100 | 98 | 97 | 94 |
| Purity by SE-UPLC | % HMW | 2.1 | 10.3 | 18.6 | 26.6 |
| | % Main | 97.2 | 88.7 | 79.0 | 70.7 |
| | % LMW | 0.7 | 1.0 | 2.4 | 2.7 |
| Charge Variant Analysis | % Acidic | 27.7 | 53.7 | 77.9 | 86.9 |
| by CEX-UPLC | % Main | 62.5 | 41.0 | 20.9 | 9.5 |
| | % Basic | 9.9 | 5.3 | 1.3 | 3.6 |
| MFI (# particles/mL) | 2-10 μm | 125 | 1428 | 1785 | 377 |
| | ≥10 μm | 23 | 29 | 209 | 19 |
| | ≥25 μm | 4 | 2 | 23 | 4 |

TABLE 19
Thermal stability of formulations with 0.01% poloxamer 188

| | | | Length of Storage at 45° C. (months) | | |
|---|---|---|---|---|---|
| Assay | | t = 0 | 1 | 2 | 3 |
| Visual Appearance | | Pass | Pass | Pass | Pass |
| Increase in Turbidity (OD 405 nm) | | — | 0.02 | 0.18 | 0.28 |
| pH | | 6.1 | 6.0 | 6.1 | 6.0 |
| % antibody Recovered by RP-UPLC | | 100 | 100 | 97 | 98 |
| Purity by SE-UPLC | % HMW | 2.1 | 9.1 | 16.3 | 23.7 |
| | % Main | 97.2 | 90.0 | 81.4 | 73.5 |
| | % LMW | 0.7 | 0.9 | 2.3 | 2.7 |
| Charge Variant Analysis | % Acidic | 28.4 | 51.7 | 75.5 | 86.6 |
| by CEX-UPLC | % Main | 62.1 | 43.1 | 21.0 | 8.9 |
| | % Basic | 9.5 | 5.3 | 3.6 | 4.5 |
| MFI (# particles/mL) | 2-10 μm | 79 | 142 | 648 | 396 |
| | ≥10 μm | 17 | 17 | 23 | 8 |
| | ≥25 μm | 0 | 6 | 2 | 0 |

TABLE 20
Thermal stability of formulations with 0.1% poloxamer 188

| | | | Length of Storage at 45° C. (months) | | |
|---|---|---|---|---|---|
| Assay | | t = 0 | 1 | 2 | 3 |
| Visual Appearance | | Pass | Pass | Pass | Pass |
| Increase in Turbidity (OD 405 nm) | | — | 0.01 | 0.14 | 0.21 |
| pH | | 6.1 | 6.0 | 6.1 | 6.0 |
| % antibody Recovered by RP-UPLC | | 100 | 99 | 97 | 100 |
| Purity by SE-UPLC | % HMW | 2.1 | 9.1 | 16.4 | 23.7 |
| | % Main | 97.3 | 90.0 | 81.2 | 73.6 |
| | % LMW | 0.6 | 0.9 | 2.4 | 2.8 |
| Charge Variant Analysis | % Acidic | 28.1 | 51.9 | 74.9 | 84.0 |
| by CEX-UPLC | % Main | 62.1 | 46.2 | 21.0 | 8.7 |
| | % Basic | 9.8 | 1.9 | 4.1 | 7.4 |
| MFI (# particles/mL) | 2-10 μm | 69 | 540 | 993 | 811 |
| | ≥10 μm | 0 | 17 | 38 | 25 |
| | ≥25 μm | 0 | 0 | 2 | 0 |

TABLE 21
Thermal stability of formulations with 0.5% poloxamer 188

| | | | Length of Storage at 45° C. (months) | | |
|---|---|---|---|---|---|
| Assay | | t = 0 | 1 | 2 | 3 |
| Visual Appearance | | Pass | Pass | Pass | Pass |
| Increase in Turbidity (OD 405 nm) | | — | 0.02 | 0.06 | 0.22 |
| pH | | 6.1 | 6.1 | 6.1 | 6.0 |
| % antibody Recovered by RP-UPLC | | 100 | 100 | 97 | 100 |
| Purity by SE-UPLC | % HMW | 2.1 | 9.1 | 16.8 | 24.2 |
| | % Main | 97.3 | 90.0 | 80.9 | 73.1 |
| | % LMW | 0.6 | 0.9 | 2.4 | 2.7 |
| Charge Variant Analysis | % Acidic | 29.5 | 50.8 | 77.1 | 86.6 |
| by CEX-UPLC | % Main | 61.5 | 44.5 | 21.5 | 9.0 |
| | % Basic | 9.1 | 4.8 | 1.4 | 4.4 |
| MFI (# particles/mL ) | 2-10 μm | 179 | 805 | 4089 | 1887 |
| | ≥10 μm | 10 | 8 | 671 | 307 |
| | ≥25 μm | 0 | 2 | 94 | 61 |

TABLE 22
Thermal stability of formulations with 0.01% PEG3350

| | | | Length of Storage at 45° C. (months) | | |
|---|---|---|---|---|---|
| Assay | | t = 0 | 1 | 2 | 3 |
| Visual Appearance | | Pass | Pass | Pass | Pass |
| Increase in Turbidity (OD 405 nm) | | — | 0.03 | 0.14 | 0.23 |
| pH | | 6.1 | 6.1 | 6.1 | 6.0 |
| % antibody Recovered by RP-UPLC | | 100 | 102 | 98 | 100 |
| Purity by SE-UPLC | % HMW | 2.1 | 9.3 | 16.5 | 23.6 |
| | % Main | 97.4 | 89.9 | 81.2 | 73.6 |
| | % LMW | 0.6 | 0.9 | 2.4 | 2.7 |
| Charge Variant Analysis | % Acidic | 29.4 | 51.6 | 77.3 | 86.4 |
| by CEX-UPLC | % Main | 62.2 | 46.1 | 21.5 | 11.9 |
| | % Basic | 8.5 | 2.4 | 1.1 | 1.8 |
| MFI (# particles/mL) | 2-10 μm | 42 | 494 | 1216 | 2085 |
| | ≥10 μm | 6 | 15 | 44 | 206 |
| | ≥25 μm | 0 | 6 | 6 | 40 |

TABLE 23
Thermal stability of formulations with 0.1% PEG3350

| | | | Length of Storage at 45° C. (months) | | |
|---|---|---|---|---|---|
| Assay | | t = 0 | 1 | 2 | 3 |
| Visual Appearance | | Pass | Pass | Pass | Pass |
| Increase in Turbidity (OD 405 nm) | | — | 0.03 | 0.15 | 0.23 |
| pH | | 6.0 | 6.0 | 6.1 | 6.1 |
| % antibody Recovered by RP-UPLC | | 100 | 97 | 92 | 92 |
| Purity by SE-UPLC | % HMW | 2.1 | 9.3 | 16.1 | 22.4 |
| | % Main | 97.4 | 89.8 | 81.6 | 74.9 |
| | % LMW | 0.6 | 0.9 | 2.4 | 2.8 |
| Charge Variant Analysis | % Acidic | 32.1 | 51.0 | 77.6 | 85.7 |
| by CEX-UPLC | % Main | 60.6 | 45.6 | 21.5 | 10.9 |
| | % Basic | 7.4 | 3.4 | 0.9 | 3.5 |
| MFI (# particles/mL) | 2-10 μm | 94 | 313 | 1541 | 2058 |
| | ≥10 μm | 2 | 15 | 90 | 159 |
| | ≥25 μm | 0 | 4 | 0 | 21 |

TABLE 24
Thermal stability of formulations with 0.5% PEG3350

| | | | Length of Storage at 45° C. (months) | | |
|---|---|---|---|---|---|
| Assay | | t = 0 | 1 | 2 | 3 |
| Visual Appearance | | Pass | Pass | Pass | Pass |
| Increase in Turbidity (OD 405 nm) | | — | 0.02 | 0.15 | 0.23 |
| pH | | 6.1 | 6.1 | 6.1 | 6.0 |
| % antibody Recovered by RP-UPLC | | 100 | 100 | 97 | 98 |
| Purity by SE-UPLC | % HMW | 2.1 | 9.2 | 16.7 | 23.9 |
| | % Main | 97.3 | 89.8 | 81.0 | 73.4 |
| | % LMW | 0.6 | 0.9 | 2.4 | 2.7 |
| Charge Variant Analysis | % Acidic | 29.7 | 52.0 | 75.6 | 83.6 |
| by CEX-UPLC | % Main | 62.0 | 45.7 | 21.3 | 10.5 |
| | % Basic | 8.4 | 2.3 | 3.1 | 6.0 |
| MFI (# particles/mL) | 2-10 μm | 121 | 1289 | 528 | 2654 |
| | ≥10 μm | 0 | 44 | 42 | 279 |
| | ≥25 μm | 0 | 25 | 15 | 31 |

CEX, cation exchange;
FCP, final concentrated pool;
HMW, high molecular weight;
LMW, low molecular weight;
MFI, Micro-Flow Imaging;
NR, not required;
OD, optical density;
RH, relative humidity;
RP, Reverse Phase;
SE, size exclusion;
UPLC, ultra-performance liquid chromatography

### Example 13. Reduction of Lipase Activity Using Agitation Stress and Heat Stress

In order to reduce esterase and lipase activity, free fatty acid particle formation, and/or polysorbate degradation in therapeutic products, particular methods for reducing esterase and lipase activity in formulated drug substance were investigated, as described in PCT/US2024/054106, and are incorporated by reference herein. It was surprisingly discovered that subjecting drug substance to stress conditions, such as agitation stress or heat stress, could be used to reduce esterase and lipase activity and increase the long-term stability of therapeutic molecules and surfactants in drug substance and subsequent drug products. Due to the superior stability of a biotherapeutic compared to an HCP lipase in a drug substance, lipases in a drug substance disproportionately inactivate, degrade and aggregate in response to stress. Therefore, stress conditions can be selected and optimized to provide for the maximum inactivation of lipases while sufficiently preserving the structure and function of a biotherapeutic. Any biotherapeutic that does form a size or charge variant can be removed prior to formulation, for example using CEX or SEC as disclosed in Example 14.

Dupilumab produced from CHO cells, referred to herein as mAb1 drug substance (DS), was subjected to agitation stress. 30 mL of 200 mg/mL mAb1 DS was used. Two 125 mL polycarbonate bottles were each filled with 10 mL of DS and agitated on an orbital shaker at 250 rpm for 0, 24, or 48 hours. The remaining 10 mL of DS were transferred to a 15 mL Falcon tube and served as the unstressed control. 2 mL of each DS was analyzed as either pre-stressed (control) DS (0 hours of agitation) or post-stressed DS (24 or 48 hours of agitation).

The control or stressed DS was used to prepare 150 mg/mL final drug substance (FDS), comprising 150 mg/mL mAb1, 20 mM histidine, 12.5 mM acetate, 5% sucrose, 0.2% high purity PS20, and 25 mM arginine HCl, at pH 5.9. The FDS was filter sterilized and then stored for 0 weeks, 4 weeks, or 8 weeks at 45° C. Storage at 45° C. was selected in order to accelerate the stability study. Physical stability of every sample collected above was analyzed using visual inspection for visible particles and aggregates, SE-UPLC for high and low molecular weight species, micro-flow imaging for subvisible particulate (2-300 μm) analysis, and CAD-UPLC for determining PS20 levels.

mAb1 drug substance subjected to the agitation stress and filtering steps showed less particle formation and greater polysorbate retention over time, as shown in Table 25. For context, the United States Pharmacopeia specificies ≤3000 particles of ≥10 um diameter per container, and ≤300 particles of ≥25 um diameter per container, as measured using micro-flow imaging. In the tables in this disclosure, CAD refers to a charged aerosol detector, CEX refers to cation exchange chromatography, FCP refers to a final concentrated pool, HMW refers to high molecular weight species, LMW refers to low molecular weight species, MFI refers to micro-flow imaging, NR indicates a measurement that is not required, OD refers to optical density, RH refers to relative humidity, SE refers to size exclusion, UPLC refers to ultra-performance liquid chromatography, and “—” indicates that no data is available.

TABLE 25
Thermal stability of formulations subjected to agitation stress

| | | Length of Agitation at 250 rpm (hours) | | | | | | | | |
|---|---|---|---|---|---|---|---|---|---|---|
| | | 0 | | | 24 | | | 48 | | |
| | | Length of Storage at 45° C. (weeks) | | | | | | | | |
| | | 0 | 4 | 8 | 0 | 4 | 8 | 0 | 4 | 8 |
| Visual Appearance | | Pass | Pass | Pass | Pass | Pass | Pass | Pass | Pass | Pass |
| Polysorbate Quantification % by CAD | | 100 | 32 | 25 | 100 | 59 | 48 | 100 | 65 | 33 |
| Purity by | % HMW | 1.7 | — | — | 16.6 | — | — | 30.9 | — | — |
| SE-UPLC | % Main | 98.3 | — | — | 83.5 | — | — | 69.1 | — | — |
| | % LMW | 0 | — | — | 0 | — | — | 0 | — | — |
| MFI (# | 2-10 μm | 1036 | 3260 | 503066 | 739 | 3955 | 7792 | 1269 | 13974 | 14415 |
| particles/mL) | ≥10 μm | 54 | 53 | 81146 | 15 | 53 | 39 | 73 | 140 | 125 |
| | ≥25 μm | 10 | 6 | 17563 | 3 | 3 | 0 | 7 | 14 | 3 |

Additional forms of stress were also investigated. mAb1 drug substance was subjected to heat stress. DS was stored at 45° C. for 0, 0.5, or 1 months in order to cause lipases to degrade, aggregate and/or inactivate. The control and stressed DS were used to prepare 150 mg/mL FDS, comprising 150 mg/mL mAb1, 20 mM histidine, 12.5 mM acetate, 500 sucrose, 0.2% high purity polysorbate 20, and 25 mM arginine HCl, at pH 5.9. The FDS was filter sterilized and then stored under conditions set forth in Table 26.

TABLE 26
Incubation/storage conditions for FDS from heat stressed DS

| Stress Condition | Length of Incubation | |
|---|---|---|
| No storage | t = 0 | |
| 45° C. | 0.5, 1, 1.5, 2 | months |
| 25° C. | 3, 6 | months |
| 5° C. | 3, 6, 12, 18, 24, 36 | months |

Physical stability of the thermally stressed and control non-stressed samples described above was analyzed using visual inspection for visible particles and aggregates, SE-UPLC for high and low molecular weight species, micro-flow imaging for subvisible particulate (2-300 m) analysis, and CAD-UPLC for determining PS20 levels. Results are set forth below in Tables 27-32. Subjecting DS to thermal stress prior to formulation resulted in a significant decrease of lipase activity, improving polysorbate recovery and decreasing free fatty acid particle formation. Non-stressed samples showed a significant increase in subvisible particles over time that was not observed in stressed samples. Subjecting DS to thermal stress did result in a significant increase in HMW level, but that level remained stable during further incubation.

TABLE 27
Stability of a formulation from DS with 0 months of thermal stress

| | | | Length of Storage at 5° C. (months) | | | | | |
|---|---|---|---|---|---|---|---|---|
| Assay | | t = 0 | 3 | 6 | 12 | 18 | 24 | 36 |
| Visual Appearance | | Pass | Pass | Pass | Pass | Pass | Pass | — |
| Polysorbate Quantification | | 100 | — | 53 | — | — | — | — |
| % by CAD | | | | | | | | |
| Purity by | % HMW | 2.2 | 2.2 | 2.5 | 2.5 | 2.6 | 2.7 | 2.8 |
| SE-UPLC | % Main | 96.6 | 96.6 | 95 | 96.6 | 96.5 | 95.9 | 95.4 |
| | % LMW | 1.2 | 1.2 | 2.5 | 0.9 | 0.9 | 1.5 | 1.8 |
| MFI (# | 2-10 μm | 264 | 12265 | 81,203 | 141583 | 243895 | 273027 | 371554 |
| particles/mL) | ≥10 μm | 20 | 2273 | 8446 | 16902 | 26739 | 3042 | 38956 |
| | ≥25 μm | 3 | 9 | 47 | 530 | 1780 | 113 | 3602 |

TABLE 28
Stability of a formulation from DS with 0.5 months of thermal stress

| | | | Length of Storage at 5° C. (months) | | | | | |
|---|---|---|---|---|---|---|---|---|
| Assay | | t = 0 | 3 | 6 | 12 | 18 | 24 | 36 |
| Visual Appearance | | Pass | Pass | Pass | Pass | Pass | Pass | — |
| Polysorbate Quantification | | 100 | — | 89 | — | — | — | — |
| % by CAD | | | | | | | | |
| Purity by | % HMW | 10.9 | 10.7 | 11 | 12 | 12 | 11.6 | 11.7 |
| SE-UPLC | % Main | 86.3 | 86.6 | 85.7 | 85.8 | 85.7 | 85.7 | 84.9 |
| | % LMW | 2.8 | 2.7 | 3.4 | 2.2 | 2.3 | 2.7 | 3.4 |
| MFI (# | 2-10 μm | 349 | 6691 | 5828 | 1794 | 3052 | 21842 | 258288 |
| particles/mL) | ≥10 μm | 16 | 216 | 61 | 34 | 222 | 3641 | 3586 |
| | ≥25 μm | 2 | 21 | 8 | 6 | 36 | 248 | 119 |

TABLE 29
Stability of a formulation from DS with 1 month of thermal stress

| | | | Length of Storage at 5° C. (months) | | | | | |
|---|---|---|---|---|---|---|---|---|
| Assay | | t = 0 | 3 | 6 | 12 | 18 | 24 | 36 |
| Visual Appearance | | Pass | Pass | Pass | Pass | Pass | Pass | — |
| Polysorbate Quantification | | 100 | — | 90 | — | — | — | — |
| % by CAD | | | | | | | | |
| Purity by | % HMW | 17.3 | 17.4 | 17.7 | 19 | 18.9 | 18.6 | 18.5 |
| SE-UPLC | % Main | 78.5 | 78.4 | 77.6 | 77.6 | 77.6 | 77.2 | 76.7 |
| | % LMW | 4.2 | 4.2 | 4.7 | 3.5 | 3.5 | 4.2 | 4.8 |
| MFI (# | 2-10 μm | 160 | 3898 | 2081 | 35220 | 4806 | 824 | 5189 |
| particles/mL) | ≥10 μm | 7 | 52 | 14 | 1017 | 119 | 39 | 109 |
| | ≥25 μm | 2 | 6 | 1 | 30 | 5 | 2 | 8 |

TABLE 30
Stability of a formulation from DS with 0 months of thermal stress

| | | | Length of Storage at | | | | | |
|---|---|---|---|---|---|---|---|---|
| | | | 25° C. (months) | | Length of Storage at 45° C. (months) | | | |
| Assay | | t = 0 | 3 | 6 | 0.5 | 1 | 1.5 | 2 |
| Visual Appearance | | Pass | Pass | Pass | Pass | — | Pass | — |
| PS-20 level (CAD-UPLC) | | 100 | — | 16 | — | — | — | 33 |
| Purity by | % HMW | 2.2 | 3 | 3.5 | 6.2 | 8.5 | 13.3 | 17.4 |
| SE-UPLC | % Main | 96.6 | 95.8 | 94.2 | 92.2 | 89.2 | 84.4 | 79.9 |
| | % LMW | 1.2 | 1.2 | 2.3 | 1.6 | 2.3 | 2.3 | 2.7 |
| MFI (# | 2-10 μm | 264 | 19609 | 146335 | 25146 | 2532 | 8226 | 263410 |
| particles/mL) | ≥10 μm | 20 | 492 | 23326 | 750 | 47 | 206 | 41033 |
| | ≥25 μm | 3 | 19 | 3825 | 60 | 5 | 20 | 9790 |

TABLE 31
Stability of a formulation from DS with 0.5 months of thermal stress

| | | | Length of Storage at | | | | | |
|---|---|---|---|---|---|---|---|---|
| | | | 25° C. (months) | | Length of Storage at 45° C. (months) | | | |
| Assay | | t = 0 | 3 | 6 | 0.5 | 1 | 1.5 | 2 |
| Visual Appearance | | Pass | Pass | Pass | Pass | — | Pass | — |
| PS-20 level (CAD-UPLC) | | 100 | — | 68 | — | — | — | 75 |
| Purity by | % HMW | 10.9 | 11 | 11.5 | 14.5 | 14.6 | 21.2 | 24.8 |
| SE-UPLC | % Main | 86.3 | 86.2 | 84.4 | 82.3 | 81 | 74.5 | 70.3 |
| | % LMW | 2.8 | 2.8 | 4.2 | 3.2 | 4.4 | 4.4 | 4.9 |
| MFI (# | 2-10 μm | 349 | 6232 | 8660 | 4838 | 9249 | 18683 | 7797 |
| particles/mL) | ≥10 μm | 16 | 52 | 169 | 60 | 172 | 426 | 272 |
| | ≥25 μm | 2 | 4 | 3 | 8 | 0 | 29 | 6 |

TABLE 32
Stability of a formulation from DS with 1 month of thermal stress

| | | | Length of Storage at | | | | | |
|---|---|---|---|---|---|---|---|---|
| | | | 25° C. (months) | | Length of Storage at 45° C. (months) | | | |
| Assay | | t = 0 | 3 | 6 | 0.5 | 1 | 1.5 | 2 |
| Visual Appearance | | Pass | Pass | Pass | Pass | — | Pass | — |
| PS-20 level (CAD-UPLC) | | 100 | — | 77 | — | — | — | 84 |
| Purity by | % HMW | 17.3 | 17.5 | 17.6 | 20.8 | 19.4 | 27 | 30.2 |
| SE-UPLC | % Main | 78.5 | 78.4 | 77.2 | 74.6 | 74.4 | 67.4 | 63.6 |
| | % LMW | 4.2 | 4.2 | 5.2 | 4.6 | 6.2 | 5.6 | 6.3 |
| MFI (# | 2-10 μm | 160 | 5799 | 11893 | 3258 | 10647 | 18440 | 4921 |
| particles/mL) | ≥10 μm | 7 | 84 | 236 | 38 | 359 | 453 | 70 |
| | ≥25 μm | 2 | 3 | 19 | 10 | 18 | 28 | 4 |

### Example 14. Depletion of High Molecular Weight Species from Stress Pre-Treated Drug Substance

As disclosed in Example 13, subjecting a drug substance to agitation stress or thermal stress can result in an increase in HMW species. Compare, for example, the 2.2% HMW species at time zero for untreated DS in Tables 27 and 30, compared to 10.9% for DS treated to 0.5 months of thermal stress in Tables 28 and 31, and 17.3% for DS treated to 1 month of thermal stress in Tables 29 and 32. HMW species may be formed from HCPs, drug protein, or a combination. HMW species can be removed using further processing steps such as filtration or chromatography.

mAb1 DS was subjected to 1 or 2 weeks of thermal stress at 45° C. or 50° C. Thermally stressed DS was subjected to cation exchange (CEX) chromatography to remove HMW species that formed during stress conditions. HMW species were efficiently depleted from stressed DS, as shown in Table 33. The HMW-depleted drug substance samples had a comparable percent of HMW species (2.3% or 2.5%) compared to a drug substance that was not subjected to stress or purification (1.7%). The percentage of HMW species was reduced to an acceptable level for a pharmaceutical composition, in particular below 5%.

TABLE 33
Clearance of HMW species from stressed drug substance

| | | | Thermal stress of 1 | | Thermal stress of 2 | |
|---|---|---|---|---|---|---|
| | | | week | | weeks | |
| Assay | | t = 0 | 45° C. | 50° C. | 45° C. | 50° C. |
| Purity by SE-UPLC before | % HMW | 1.7 | 10.2 | 26.2 | 17.0 | 33.6 |
| CEX purification | % Main | 98.3 | 89.8 | 73.8 | 83.0 | 66.4 |
| Purity by SE-UPLC after | % HMW | 1.3 | 2.3 | 2.3 | 2.5 | 2.5 |
| CEX purification | % Main | 98.7 | 97.7 | 97.7 | 97.5 | 97.5 |

HMW-depleted DS was formulated into FDS comprising 150 mg/mL mAb1, 20 mM histidine, 12.5 mM acetate, 5% sucrose, 0.2% PS80, and 25 mM arginine HCl, at pH 5.9. The stability of FDS from HMW-depleted thermally stressed DS was compared to FDS from HMW-depleted non-stressed DS. In particular, non-stressed DS (Table 33) was compared with DS stressed at 45° C. for 1 week (Table 35), 50° C. for 1 week (Table 36), 45° C. for 2 weeks (Table 37), and 50° C. for 2 weeks (Table 38). Each FDS was stored at 1, 3, or 6 months at 25° C., or 1 week, 2 weeks, 1 month, or 3 months at 45° C. before analysis. Visual appearance, turbidity, pH, PS80 recovery, purity as measured by SE-UPLC, charge variants, mAb1 recovery, number of particles/mL≥10 μm in size, and number of particles/mL≥25 μm in size were characterized.

As shown in Table 34, the FDS from control DS shows a substantial loss in PS80 after 3 months of storage, down to 50% after storage at 25° C. and 47% after storage at 45° C., illustrating the significance of lipase activity in the FDS sample. A loss of 50% of PS80 would typically be considered unacceptable for a pharmaceutical composition. Accordingly, the number of particles/mL counted by microflow imaging also increased greatly over time, to as high as 815 particles/mL≥10 μm after 3 months of storage at 25° C. PS80 degradation continued after 6 months of storage at 25° C., with the percent of PS80 recovered decreasing to 43%. The number of particles/mL measured remained high. It should be noted that, because particles may form and break apart dynamically, it is not surprising that the particle count does not linearly increase, while remaining high from 3 to 6 months.

As shown in Table 35, the FDS from DS pre-treated at 45° C. for 1 week shows a substantially different profile. After 3 months of storage, PS80 levels remained at 90% at 25° C. and 85% at 45° C., and remained at 83% after 6 months at 25° C. A loss of less than 20% of PS80 may be considered acceptable for a pharmaceutical composition. The number of particles/mL remained low after 3 months and 6 months of storage, only increasing to 21 particles/mL≥10 μm and 2 particles/mL≥25 μm after 3 months of storage at 45° C., which is within the range of background noise for this assay (below about 100). Therefore, even the mildest pre-treatment condition showed marked improvement compared to the non-stressed control in terms of polysorbate retention, reduction of lipase activity, and reduction of particle formation.

As shown in Table 36, the FDS from DS pre-treated at 50° C. for 1 week shows even higher PS80 stability. About 96% and 94% of PS80 was recovered after 3 months of storage at 25° C. and 45° C., respectively, and about 98% after 6 months of storage at 25° C. Given a margin of error of about 10% using this assay, this is statistically indistinguishable from full recovery at all time points and storage conditions. The number of particles/mL remained low, within the range of background noise. Overall, the stability of the FDS from DS pre-treated at 50° C. for 1 week is similar to what would be expected in an FDS with no lipase contamination at all.

As shown in Table 37, the FDS from DS pre-treated at 45° C. for 2 weeks shows improved polysorbate stability similar to that seen from the pre-treatment at 45° C. for 1 week condition, with a recovery of PS80 above 90% even after 6 months. Particle data was not collected from the samples pre-treated with heat stress for 2 weeks due to a low amount of sample remaining.

As shown in Table 38, the FDS from DS pre-treated to 50° C. for 2 weeks shows effectively no lipase activity and complete polysorbate stability, with 100% of PS80 measured as recovered after 3 months of storage at 25° C., and 97% (within measurement error of 100%) after 6 months.

mAb1 stability (in terms of size variants and charge variants) and recovery were not significantly impacted by the DS treatment over the time frame of these experiments, as was expected over a short time frame. However, the dramatic loss of polysorbate seen in the control condition (Table 34) may be expected to result in a subsequent loss of mAb1 stability over the course of long-term storage.

It should be noted that the CEX step following stress pre-treatment may also deplete remaining HCP lipases from each of the samples in addition to non-functional HVIW species. This feature may be masking an even greater difference in lipase activity between stressed and non-stressed DS attributable to thermal stress alone.

TABLE 34
Thermal stability of a formulation from HMW-depleted control DS

| | | | Length of Storage at | | | | | | |
|---|---|---|---|---|---|---|---|---|---|
| | | | 25° C. (months) | | | Length of Storage at 45° C. (months) | | | |
| Assay | | t = 0 | 1 | 3 | 6 | 0.25 | 0.5 | 1 | 3 |
| Visual Appearance | | Pass | Pass | Pass | Pass | Pass | Pass | Pass | Pass |
| Increase in Turbidity (OD 405 nm) | | 0.00 | 0.01 | 0.03 | 0.08 | 0.03 | 0.07 | 0.09 | 0.29 |
| pH | | 6.2 | 6.3 | 6.2 | 6.2 | 6.2 | 6.1 | 6.3 | 6.2 |
| PS-80 level (CAD-UPLC) | | 100 | 68 | 50 | 43 | 68 | 59 | 59 | 47 |
| Purity by | % HMW | 2.2 | 2.7 | 3.0 | 3.8 | 4.0 | 4.9 | 7.3 | 24.5 |
| SE-UPLC | % Main | 97.7 | 97.1 | 95.6 | 95.6 | 95.7 | 94.5 | 92.2 | 64.8 |
| | % LMW | 0.2 | 0.2 | 1.4 | 0.6 | 0.3 | 0.6 | 0.4 | 10.7 |
| Charge Variant | % Acidic | 26.9 | 29.5 | 32.8 | 37.6 | 34.5 | 46.9 | 50.2 | 59.5 |
| Analysis by CEX- | % Main | 72.8 | 68.8 | 64.9 | 59.4 | 61.6 | 49.3 | 48.4 | 39.6 |
| UPLC | % Basic | 0.3 | 1.7 | 2.3 | 3.0 | 4.0 | 3.9 | 1.4 | 0.9 |
| % mAb1 recovered by RP-UPLC | | 100 | 103 | 99 | 101 | 101 | 98 | 99 | 97 |
| MFI (# particles/mL) | ≥10 μm | 38 | 13 | 815 | 108 | 31 | 13 | 10 | 696 |
| | ≥25 μm | 2 | 2 | 2 | 8 | 4 | 7 | 4 | 42 |

TABLE 35
Thermal stability of a formulation from HMW-depleted DS pre-treated at 45° C. for 1 week

| | | | Length of Storage at | | | | | | |
|---|---|---|---|---|---|---|---|---|---|
| | | | 25° C. (months) | | | Length of Storage at 45° C. (months) | | | |
| Assay | | t = 0 | 1 | 3 | 6 | 0.25 | 0.5 | 1 | 3 |
| Visual Appearance | | Pass | Pass | Pass | Pass | Pass | Pass | Pass | Pass |
| Increase in Turbidity (OD 405 nm) | | 0.00 | 0.00 | 0.02 | 0.06 | 0.01 | 0.05 | 0.07 | 0.27 |
| pH | | 6.2 | 6.3 | 6.2 | 6.2 | 6.2 | 6.2 | 6.3 | 6.2 |
| PS-80 level (CAD-UPLC) | | 100 | 98 | 90 | 83 | 97 | 86 | 94 | 85 |
| Purity by | % HMW | 2.7 | 3.0 | 3.4 | 3.9 | 3.8 | 4.7 | 6.5 | 21.5 |
| SE-UPLC | % Main | 97.0 | 96.6 | 95.4 | 95.2 | 95.9 | 94.4 | 92.9 | 56.6 |
| | % LMW | 0.3 | 0.4 | 1.2 | 0.9 | 0.3 | 1.0 | 0.6 | 21.9 |
| Charge Variant | % Acidic | 18.0 | 34.1 | 38.0 | 41.7 | 38.8 | 48.0 | 53.9 | 65.6 |
| Analysis by CEX- | % Main | 76.3 | 60.9 | 60.0 | 56.8 | 56.2 | 47.9 | 44.7 | 33.8 |
| UPLC | % Basic | 5.7 | 5.0 | 2.0 | 1.5 | 5.0 | 4.2 | 1.4 | 0.7 |
| % mAb1 recovered by RP-UPLC | | 100 | 102 | 99 | 102 | 103 | 100 | 99 | 98 |
| MFI (# particles/mL) | ≥10 μm | 2 | 10 | 2 | 19 | 23 | 8 | 10 | 21 |
| | ≥25 μm | 0 | 2 | 2 | 0 | 2 | 0 | 2 | 2 |

TABLE 36
Thermal stability of a formulation from HMW-depleted DS pre-treated at 50° C. for 1 week

| | | | Length of Storage at | | | | | | |
|---|---|---|---|---|---|---|---|---|---|
| | | | 25° C. (months) | | | Length of Storage at 45° C. (months) | | | |
| Assay | | t = 0 | 1 | 3 | 6 | 0.25 | 0.5 | 1 | 3 |
| Visual Appearance | | Pass | Pass | Pass | Pass | Pass | Pass | Pass | Pass |
| Increase in Turbidity (OD 405 nm) | | 0.00 | 0.0 | 0.01 | 0.04 | 0.0 | 0.03 | 0.03 | 0.14 |
| pH | | 6.1 | 6.1 | 6.0 | 6.1 | 6.0 | 6.0 | 6.1 | 6.0 |
| PS-80 level (CAD-UPLC) | | 100 | 101 | 96 | 98 | 101 | 93 | 101 | 94 |
| Purity by | % HMW | 3.0 | 3.3 | 3.5 | 4.1 | 4.0 | 4.3 | 6.2 | 18.9 |
| SE-UPLC | % Main | 96.7 | 96.2 | 95.3 | 95.0 | 95.7 | 94.6 | 93.2 | 70.4 |
| | % LMW | 0.3 | 0.5 | 1.2 | 0.9 | 0.3 | 1.1 | 0.6 | 10.7 |
| Charge Variant | % Acidic | 30.5 | 36.1 | 38.7 | 42.2 | 41.0 | 53.9 | 54.8 | 60.1 |
| Analysis by CEX- | % Main | 62.8 | 61.1 | 59.3 | 56.3 | 53.7 | 43.7 | 43.5 | 38.9 |
| UPLC | % Basic | 6.7 | 2.8 | 2.0 | 1.4 | 5.4 | 2.3 | 1.8 | 1.1 |
| % mAb1 recovered by RP-UPLC | | 100 | 102 | 99 | 101 | 103 | 95 | 99 | 97 |
| MFI (# particles/mL) | ≥10 μm | 27 | 21 | 35 | 77 | — | 19 | 13 | 2 |
| | ≥25 μm | 8 | 4 | 6 | 27 | — | 4 | 2 | 0 |

TABLE 37
Thermal stability of a formulation from HMW-depleted DS pre-treated at 45° C. for 2 weeks

| | | | Length of Storage at 25° C. | | | | | | |
|---|---|---|---|---|---|---|---|---|---|
| | | | (months) | | | Length of Storage at 45° C. (months) | | | |
| Assay | | t = 0 | 1 | 3 | 6 | 0.25 | 0.5 | 1 | 3 |
| Visual Appearance | | Pass | Pass | Pass | Pass | Pass | Pass | Pass | Pass |
| Increase in Turbidity (OD 405 nm) | | 0.00 | 0.02 | 0.03 | 0.03 | 0.03 | 0.01 | 0.01 | 0.24 |
| pH | | 6.1 | 6.2 | 6.1 | 6.2 | 6.1 | 61. | 6.1 | 6.2 |
| PS-80 level (CAD-UPLC) | | 100 | 92 | 96 | 92 | 91 | 95 | 88 | 91 |
| Purity by | % HMW | 2.9 | 3.1 | 3.4 | 3.9 | 5.5 | 4.1 | 9.5 | 19.2 |
| SE-UPLC | % Main | 96.5 | 96.0 | 95.3 | 95.1 | 94.1 | 95.0 | 86.2 | 71.7 |
| | % LMW | 0.6 | 0.9 | 1.4 | 1.0 | 0.4 | 0.9 | 4.4 | 9.2 |
| Charge Variant | % Acidic | 36.4 | 38.9 | 41.6 | 45.7 | 59.1 | 46.0 | 58.8 | 58.8 |
| Analysis by CEX- | % Main | 56.4 | 58.7 | 56.4 | 51.7 | 39.3 | 52.3 | 39.7 | 39.7 |
| UPLC | % Basic | 7.3 | 2.4 | 2.1 | 2.6 | 1.6 | 1.7 | 1.5 | 1.5 |

| | | | | | | | | |
|---|---|---|---|---|---|---|---|---|
| % mAb1 recovered by RP-UPLC | 100 | 98 | 99 | 102 | 107 | 103 | 101 | 99 |

TABLE 38
Thermal stability of a formulation from HMW-depleted DS pre-treated at 50° C. for 2 weeks

| | | | Length of Storage at 25° C. | | | Length of Storage at 45° C. | | | |
|---|---|---|---|---|---|---|---|---|---|
| | | | (months) | | | (months) | | | |
| Assay | | t = 0 | 1 | 3 | 6 | 0.25 | 0.5 | 1 | 3 |
| Visual Appearance | | Pass | Pass | Pass | Pass | Pass | Pass | Pass | Pass |
| Increase in Turbidity (OD 405 nm) | | 0.00 | 0.09 | 0.03 | 0.03 | 0.03 | 0.01 | 0.07 | 0.22 |
| pH | | 6.0 | 6.2 | 6.1 | 6.2 | 6.1 | 6.1 | 6.1 | 6.1 |
| PS-80 level (CAD-UPLC) | | 100 | 94 | 100 | 97 | 91 | 99 | 94 | 95 |
| Purity by SE-UPLC | % HMW | 2.8 | 3.0 | 3.1 | 3.7 | 5.1 | 3.9 | 8.2 | 17.8 |
| | % Main | 96.5 | 96.1 | 95.3 | 95.0 | 94.4 | 95.1 | 85.9 | 69.6 |
| | % LMW | 0.7 | 1.0 | 1.7 | 1.3 | 0.5 | 1.0 | 5.9 | 12.6 |
| Charge Variant | % Acidic | 42.4 | 42.6 | 47.2 | 50.7 | 43.8 | 51.0 | 60.9 | 65.2 |
| Analysis by CEX- | % Main | 50.6 | 54.7 | 51.1 | 46.6 | 53.9 | 46.8 | 35.3 | 33.8 |
| UPLC | % Basic | 7.0 | 2.7 | 1.7 | 2.7 | 2.3 | 2.2 | 3.8 | 1.0 |

| | | | | | | | | |
|---|---|---|---|---|---|---|---|---|
| % mAb1 recovered by RP-UPLC | 100 | 97 | 99 | 101 | 119 | 104 | 101 | 100 |

These results demonstrate that the disclosed methods of subjecting drug substance to agitation stress or heat stress, optionally followed by HMW depletion, produce an improved pharmaceutical composition with reduced lipase activity, reduced polysorbate degradation, and reduced free fatty acid particle formation.

The present invention is not to be limited in scope by the specific aspects described herein. Indeed, various modifications of the invention in addition to those described herein will become apparent to those skilled in the art from the foregoing description. Such modifications are intended to fall within the scope of the appended claims.

The following Enumerated Examples set forth below provide additional aspects of the present disclosure.
1. A method for producing a recombinant protein, comprising:
   (a) culturing cells expressing a recombinant protein in a seed train;
   (b) transferring cells from each vessel of said seed train to the next vessel at a predetermined time point;
   (c) transferring cells from the final vessel of said seed train to a production bioreactor at a predetermined time point; and
   (d) isolating the recombinant protein from the production bioreactor.
2. The method of Enumerated Example 1, wherein said recombinant protein is selected from a group consisting of an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, a variant thereof, a fragment thereof, and a multimer thereof.
3. The method of Enumerated Example 1, wherein said recombinant protein is dupilumab.
4. The method of any one of Enumerated Examples 1-3, wherein said final vessel is a 3,000 L bioreactor.
5. The method of any one of Enumerated Examples 1-4, wherein the predetermined time point of step (c) is from 60 hours to 80 hours, from 65 hours to 75 hours, from 65 hours to 72.5 hours, from 67 hours to 70 hours, from 68 hours to 69 hours, or about 68.5 hours between inoculation of said final vessel and inoculation of said production bioreactor.
6. The method of Enumerated Example 5, wherein said predetermined time point is about 68.5 hours.
7. The method of any one of Enumerated Examples 1-6, wherein said seed train comprises a 500 L bioreactor at N-2.
8. The method of Enumerated Example 7, wherein a predetermined time point for said 500 L bioreactor is from 50 to 52 hours or about 51 hours between inoculation of said 500 L bioreactor and inoculation of the next vessel in the seed train.
9. The method of any one of Enumerated Examples 1-8, wherein said seed train comprises a 50 L bioreactor at N-3.
10. The method of Enumerated Example 9, wherein a predetermined time point for said 50 L bioreactor is from 73 to 75 hours or about 74 hours between inoculation of said 50 L bioreactor and inoculation of the next vessel in the seed train.
11. The method of any one of Enumerated Examples 1-10, wherein said seed train comprises a 20 L bioreactor at N-4.
12. The method of Enumerated Example 11, wherein a predetermined time point for said 20 L bioreactor is from 73 to 75 hours or about 74 hours between inoculation of said 20 L bioreactor and inoculation of the next vessel in the seed train.
13. The method of any one of Enumerated Examples 1-12, wherein said seed train comprises a 2 L bioreactor at N-5.
14. The method of Enumerated Example 13, wherein a predetermined time point for said 2 L bioreactor is from 47.5 to 48.5 hours or about 48 hours between inoculation of said 2 L bioreactor and inoculation of the next vessel in the seed train.
15. The method of any one of Enumerated Examples 1-14, wherein said seed train comprises a 500 mL shake flask at N-6.
16. The method of Enumerated Example 15, wherein a predetermined time point for said 500 mL shake flask is from 79 to 80.5 hours or about 80 hours between inoculation of said 500 mL shake flask and inoculation of the next vessel in the seed train.
17. The method of any one of Enumerated Examples 1-16, wherein said production bioreactor is a 10,000 L bioreactor.
18. The method of any one of Enumerated Examples 1-17, wherein a titer of said recombinant protein is increased compared to a titer of a recombinant protein produced using a method wherein cells from each vessel of said seed train are transferred to the next vessel at a variable time point.
19. The method of any one of Enumerated Examples 1-18, wherein said cells are selected from the group consisting of CHO cells, HEK293 cells, and BHK cells.
20. The method of Enumerated Example 19, wherein said CHO cells are selected from the group consisting of CHO-K1, CHO DUX B-11, Veggie-CHO, GS-CHO, S-CHO, or CHO lec.
21. The method of any one of Enumerated Examples 1-20, wherein said cells of step (c) have a viable cell density from 40-50×10⁵ viable cells/mL, from 45-50×10⁵ viable cells/mL, from 46-48×10⁵ viable cells/mL, from 46.5-47.5×10⁵ viable cells/mL, from 46.9-47.1×10⁵ viable cells/mL, about 40×10⁵ viable cells/mL, about 45×10⁵ viable cells/mL, about 47×10⁵ viable cells/mL, or about 50×10⁵ viable cells/mL.
22. The method of any one of Enumerated Examples 1-21, further comprising determining said predetermined time points by selecting time points for each vessel of said seed train based on the achievement of optimized viable cell densities for each vessel of said seed train, wherein optimized viable cell densities are optimized for increased protein production from a production bioreactor.
23. A method of culturing cells that produce a recombinant protein using a seed train, wherein the method comprises the steps of:
   (a) culturing cells in a first bioreactor (N-5) for a predetermined period of time;
   (b) transferring the cells from the first bioreactor to a second bioreactor (N-4) and culturing the cells for a predetermined period of time;
   (c) transferring the cells from the second bioreactor to a third bioreactor (N-3) and culturing the cells for a predetermined period of time;
   (d) transferring the cells from the third bioreactor to a fourth bioreactor (N-2) and culturing the cells for a predetermined period of time; and
   (e) transferring the cells from the fourth bioreactor to a fifth bioreactor (N-1) and culturing the cells for a predetermined period of time,
   wherein the cells express the recombinant protein, and the cells are transferred to a production bioreactor.
24. The method according to Enumerated Example 23, further comprising the steps of culturing cells in a shake flask (N-6) for a predetermined period of time, and transferring the cells to the first bioreactor.
25. The method according to Enumerated Example 23, wherein the first bioreactor is a 2 L bioreactor and the predetermined period of time is 47.5 to 48.5 hours.
26. The method according to Enumerated Example 23, wherein the first bioreactor is a 2 L bioreactor and the predetermined period of time is 48 hours.
27. The method according to Enumerated Example 23, wherein the second bioreactor is a 20 L bioreactor and the predetermined period of time is 73 to 75 hours.
28. The method according to Enumerated Example 23, wherein the second bioreactor is a 20 L bioreactor and the predetermined period of time is 74 hours.
29. The method according to Enumerated Example 23, wherein the third bioreactor is a 50 L bioreactor and the predetermined period of time is 73 to 75 hours.
30. The method according to Enumerated Example 23, wherein the third bioreactor is a 50 L bioreactor and the predetermined period of time is 74 hours.
31. The method according to Enumerated Example 23, wherein the fourth bioreactor is a 500 L bioreactor and the predetermined period of time is 50 to 52 hours.
32. The method according to Enumerated Example 23, wherein the fourth bioreactor is a 500 L bioreactor and the predetermined period of time is 51 hours.
33. The method according to Enumerated Example 23, wherein the fifth bioreactor is a 3000 L bioreactor and the predetermined period of time is 65 to 72.5 hours.
34. The method according to Enumerated Example 23, wherein the fifth bioreactor is a 3000 L bioreactor and the predetermined period of time is 68.5 hours.
35. The method according to Enumerated Example 24, wherein the shake flask is a 500 mL shake flask and the predetermined period of time is 79 to 80.5 hours.
36. The method according to Enumerated Example 24, wherein the shake flask is a 500 mL shake flask and the predetermined period of time is 80 hours.
37. The method according to Enumerated Example 23, wherein a viable cell density is achieved and is one selected from the group consisting of 40-50×10⁵ viable cells/mL, 45-50×10⁵ viable cells/mL, 46-48×10⁵ viable cells/mL, 46.5-47.5×10⁵ viable cells/mL, 46.9-47.1×10⁵ viable cells/mL, about 40×10⁵ viable cells/mL, about 45×10⁵ viable cells/mL, about 47×10⁵ viable cells/mL, and about 50×10⁵ viable cells/mL.
38. The method according to Enumerated Example 23 or 24, wherein the production bioreactor is a 10,000 L bioreactor.
39. The method according to Enumerated Example 38, wherein the recombinant protein is dupilumab.
40. The method according to Enumerated Example 39, wherein the method provides increased production of dupilimab per 10,000 L working volume as compared to a method based upon target viable cell density, wherein the increase is selected from the group of ranges consisting of 0.1 to 2.5 kg, 0.5 to 2.5 kg, 0.5 to 2 kg, 0.6 to 2 kg, 0.6 to 1.9 kg, 0.7 to 1.8 kg, 0.7 to 1.7 kg, 0.7 to 1.6 kg, 0.8 to 1.6 kg, 0.8 to 1.5 kg, 0.9 to 1.5 kg, 0.9 to 1.4 kg. 0.9 to 1.3 kg, 1 to 1.5 kg, 1 to 1.4 kg, 1 to 1.3 kg, 1 to 1.2 kg, 1 to 1.1 kg, and 1.1 to 1.2 kg.
41. The method according to Enumerated Example 40, wherein the method provides an average increase of 1.1 kg of dupilumab per 10,000 L working volume as compared to a method based upon target viable cell density.
42. A method of culturing cells that produce dupilumab, wherein the method comprises the steps of:
   (a) culturing cells in a 500 mL shake flask (N-6) for 79 to 80.5 hours;
   (b) transferring the cells from the 500 mL shake flask to a 2 L bioreactor (N-5) and culturing the cells for 47.5 to 48.5 hours;
   (c) transferring the cells from the 2 L bioreactor to a 20 L bioreactor (N-4) and culturing the cells for 73 to 75 hours;
   (d) transferring the cells from the 20 L bioreactor to a 50 L bioreactor (N-3) and culturing the cells for 73 to 75 hours;
   (e) transferring the cells from the 50 L bioreactor to a 500 L bioreactor (N-2) and culturing the cells for 50 to 52 hours;
   (f) transferring the cells from the 500 L bioreactor to a 3000 L bioreactor (N-1) and culturing the cells for 65 to 72.5 hours; and
   (g) transferring the cells from the 3000 L bioreactor to a production bioreactor.
43. The method according to Enumerated Example 42, wherein the 500 mL shake flask (N-6) is cultured for 80 hours.
44. The method according to Enumerated Example 42, wherein the 2 L bioreactor is cultured for 48 hours.
45. The method according to Enumerated Example 42, wherein the 20 L bioreactor is cultured for 74 hours.
46. The method according to Enumerated Example 42, wherein the 50 L bioreactor is cultured for 74 hours.
47. The method according to Enumerated Example 42, wherein the 500 L bioreactor is cultured for 51 hours.
48. The method according to Enumerated Example 42, wherein the 3000 L bioreactor is cultured for 68.5 hours.
49. The method according to Enumerated Examples 42-48, wherein the method provides increased production of dupilimab per 10,000 L working volume as compared to a method based upon target viable cell density, wherein the increase is selected from the group of ranges consisting of 0.1 to 2.5 kg, 0.5 to 2.5 kg, 0.5 to 2 kg, 0.6 to 2 kg, 0.6 to 1.9 kg, 0.7 to 1.8 kg, 0.7 to 1.7 kg, 0.7 to 1.6 kg, 0.8 to 1.6 kg, 0.8 to 1.5 kg, 0.9 to 1.5 kg, 0.9 to 1.4 kg. 0.9 to 1.3 kg, 1 to 1.5 kg, 1 to 1.4 kg, 1 to 1.3 kg, 1 to 1.2 kg, 1 to 1.1 kg, and 1.1 to 1.2 kg.
50. The method according to Enumerated Example 49, wherein the method provides an average increase of 1.1 kg of dupilumab per 10,000 L working volume as compared to a method based upon target viable cell density.
51. A seed train system that performs the methods according to Enumerated Examples 1-41.
52. Dupilumab produced by the methods of Enumerated Examples 1-50.
53. Dupilumab produced by the seed train system of Enumerated Example 51.
54. A method of reducing lipase activity in a pharmaceutical composition comprising dupilumab, wherein the method comprises subjecting dupilumab produced by the method of Enumerated Examples 1-50 and a lipase to anion exchange chromatography, wherein a pH of the dupilumab loaded to the anion exchange chromatography column is between about 7.8 and about 8.3.
55. A method of reducing lipase activity in a pharmaceutical composition comprising dupilumab, wherein the method comprises subjecting dupilumab produced according to Enumerated Examples 1-50 and a lipase to stress conditions to form a sample with inactivated lipase; and formulating the sample with inactivated lipase to produce a pharmaceutical composition comprising dupilimab with reduced lipase activity.
56. The method according to Enumerated Examples 54 or 55, wherein the formulating step comprises adding a fatty acid ester to the sample, wherein the fatty acid ester is polysorbate 20, polysorbate 40, polysorbate 60, or polysorbate 80.
57. The method according to Enumerated Example 56, wherein the fatty acid ester is polysorbate 80 and a concentration of oleic acid esters in the polysorbate 80 is at least 80%.
58. The method according to Enumerated Example 57, wherein a concentration of oleic acid esters in the polysorbate 80 is at least 98% or at least 99%.
59. The method according to Enumerated Examples 54-58, wherein the stress conditions include agitation stress and/or heat stress.
60. The method according to Enumerated Example 59, wherein the agitation stress comprises shaking the sample at from 50 to 500 rpm, from 200 to 300 rpm, about 50 rpm, about 75 rpm, about 100 rpm, about 125 rpm, about 150 rpm, about 200 rpm, about 225 rpm, about 250 rpm, about 275 rpm, about 300 rpm, about 325 rpm, about 350 rpm, about 375 rpm, about 400 rpm, about 425 rpm, about 450 rpm, about 475 rpm, or about 500 rpm.
61. The method according to Enumerated Example 59, wherein the agitation stress comprises shaking the sample for from 1 to 96 hours, from 24 to 48 hours, about 1 hour, about 2 hours, about 3 hours, about 4 hours, about 5 hours, about 6 hours, about 12 hours, about 18 hours, about 24 hours, about 30 hours, about 36 hours, about 42 hours, about 48 hours, about 60 hours, about 72 hours, about 84 hours, or about 96 hours.
62. The method according to Enumerated Example 59, wherein the heat stress comprises storing the sample at from about 30° C. to about 60° C., from about 35° C. to about 55° C., from about 40° C. to about 50° C., from about 44° C. to about 46° C., about 30° C., about 35° C., about 40° C., about 45° C., about 50° C., about 55° C., or about 60° C.
63. The method of Enumerated Example 62, wherein the storing is from 1 day to 6 months, from 3 days to 3 months, from 1 week to 2 months, from 0.5 months to 1 month, about 1 day, about 2 days, about 3 days, about 1 week, about 2 weeks, about 0.5 months, about 3 weeks, about 4 weeks, about 1 month, about 5 weeks, about 6 weeks, about 7 weeks, about 8 weeks, about 2 months, about 3 months, about 3.5 months, about 4 months, about 4.5 months, about 5 months, about 5.5 months, or about 6 months.
64. The method of any one of Enumerated Examples 54-63, further comprising subjecting the sample with inactivated lipase to filtration, enrichment, or chromatographic separation to remove high molecular weight (HMW) species prior to step (b).
65. The method of Enumerated Example 64, wherein the chromatographic separation comprises cation exchange chromatography.
66. The method of Enumerated Example 64, wherein the chromatographic separation comprises size exclusion chromatography.
67. The method of any one of Enumerated Examples 54-66, wherein the formulating step comprises adding excipients to the sample.
68. A method for producing a pharmaceutical composition comprising dupilumab, wherein the pharmaceutical composition has reduced lipase activity and the method comprises the steps of:
   (a) subjecting dupilumab produced according to Enumerated Examples 1-50 to affinity chromatography;
   (b) subjecting dupilumab pooled from eluate of step (a) to viral inactivation at a pH from about 3 to about 4 and then adjusting the pH to from about 5 to about 8;
   (c) subjecting dupilumab pooled from step (b) to anion exchange chromatography in flowthrough mode;
   (d) subjecting dupilumab pooled from eluate of step (c) to hydrophobic interaction chromatography in flowthrough mode;
   (e) subjecting dupilumab pooled from flowthrough fractions of step (d) to virus retentive filtration;
   (f) subjecting a sample from step (e) including dupilumab and a lipase to agitation stress or heat stress to form a pharmaceutical composition with reduced lipase activity.
69. A method for producing a pharmaceutical composition comprising dupilumab, wherein the pharmaceutical composition has reduced lipase activity and the method comprises the steps of:
   (a) subjecting harvested dupilumab produced according to Enumerated Examples 1-50 to affinity chromatography;
   (b) subjecting the dupilumab pooled from eluate of step (a) to viral inactivation at a pH from about 3 to about 4 and then adjusting the pH to from about 5 to about 8;
   (c) subjecting the dupilumab pooled from step (b) to anion exchange chromatography in flowthrough mode;
   (d) subjecting the dupilumab pooled from flowthrough fractions of step (c) to virus retentive filtration;
   (e) subjecting a sample from step (d) including Dupilumab and a lipase to agitation stress or heat stress to form a pharmaceutical composition with reduced lipase activity.
70. The method of Enumerated Examples 68 or 69, further comprising subjecting dupilumab to filtration, enrichment, or chromatographic separation to remove high molecular weight species.
71. The method of Enumerated Example 70, wherein the chromatographic separation comprises ion exchange chromatography, cation exchange chromatography, or size exclusion chromatography.
72. The method of any one of Enumerated Examples 68-71, wherein a pH of the sample loaded to the AEX column is between about 7.8 and about 8.3.
73. A method for producing a dupilumab pharmaceutical composition with increased stability, comprising:
   reducing lipase activity in a composition by subjecting dupilumab produced according to Enumerated Examples 1-50 and a lipase to anion exchange (AEX) chromatography, wherein a pH of the sample loaded to the AEX column is between about 7.8 and about 8.3.
74. The method of Enumerated Example 73, further comprising the step of subjecting dupilumab after AEX chromatography to agitation stress or heat stress to reduce lipase activity.
75. The method of any one of Enumerated Examples 68-74, further comprising the addition of poloxamer 188 or PEG3350 to produce a formulation comprising a dupilumab pharmaceutical composition with reduced lipase activity and increased stability.
76. The method of Enumerated Example 75, wherein the formulation is substantially free of polysorbate.
77. The method of Enumerated Example 75 or 76, wherein a concentration of the poloxamer 188 is from 0.005% to 5%, from 0.05% to 2.5%, from 0.1% to 1%, about 0.05%, about 0.01%, about 0.02%, about 0.03%, about 0.04%, about 0.05%, about 0.06%, about 0.07%, about 0.08%, about 0.09%, about 0.1%, about 0.15%, about 0.2%, about 0.25%, about 0.3%, about 0.4%, about 0.5%, about 1%, about 1.5%, about 2%, about 2.5%, about 3%, about 3.5%, about 4%, about 4.5%, or about 5%.
78. The method of Enumerated Example 75 or 76, wherein a concentration of the PEG3350 is from 0.005% to 5%, from 0.05% to 2.5%, from 0.1% to 1%, about 0.05%, about 0.01%, about 0.02%, about 0.03%, about 0.04%, about 0.05%, about 0.06%, about 0.07%, about 0.08%, about 0.09%, about 0.1%, about 0.15%, about 0.2%, about 0.25%, about 0.3%, about 0.4%, about 0.5%, about 1%, about 1.5%, about 2%, about 2.5%, about 3%, about 3.5%, about 4%, about 4.5%, or about 5%. 79. A method for producing a dupilumab pharmaceutical composition with increased stability, comprising:
   (a) subjecting harvested dupilumab according to Enumerated Examples 1-50 to anion exchange chromatography in flowthrough mode;
   (b) subjecting flowthrough fractions from step (a) to hydrophobic interaction chromatography in flowthrough mode; and
   (c) formulating dupilumab isolated from step (b) with polysorbate 80, wherein a concentration of oleic acid esters in the polysorbate 80 is at least 80%.
80. The method of Enumerated Example 79, wherein a concentration of the oleic acid esters is at least 98% or at least 99%.
81. The method of any one of Enumerated Examples 68 to 80, wherein a pH of the dupilumab produced according to Enumerated Examples 1-50 that is loaded to the anion exchange chromatography column is from about 7.8 to about 8.3, from about 7.9 to about 8.2, about 7.8, about 7.9, about 8.0, about 8.1, about 8.2, or about 8.3.
82. A method for producing a dupilumab pharmaceutical composition with reduced lipase activity, comprising:
   (a) subjecting a sample including harvested dupilumab according to Enumerated Examples 1-50 and a lipase to stress conditions to form a sample with inactivated lipase;
   (b) subjecting said sample with inactivated lipase to a purification step to form a high molecular weight (HMW)-depleted sample; and
   (c) formulating said HMW-depleted sample with at least one fatty acid ester to produce a dupilumab pharmaceutical composition with reduced lipase activity,
   wherein said dupilumab pharmaceutical composition has reduced lipase activity compared to a dupilumab pharmaceutical composition formulated from a sample that was not subjected to stress conditions, and wherein said reduced lipase activity is measured after a period of storage at long-term storage conditions or accelerated storage conditions of at least 2 weeks.
83. The method of Enumerated Example 82, wherein said stress conditions include agitation stress and/or heat stress.
84. The method of Enumerated Example 83, wherein said agitation stress comprises shaking said sample at from 50 to 500 rpm, from 200 to 300 rpm, about 50 rpm, about 75 rpm, about 100 rpm, about 125 rpm, about 150 rpm, about 200 rpm, about 225 rpm, about 250 rpm, about 275 rpm, about 300 rpm, about 325 rpm, about 350 rpm, about 375 rpm, about 400 rpm, about 425 rpm, about 450 rpm, about 475 rpm, or about 500 rpm.
85. The method of Enumerated Example 83 or 84, wherein said agitation stress comprises shaking said sample for from 1 to 96 hours, from 24 to 48 hours, about 1 hour, about 2 hours, about 3 hours, about 4 hours, about 5 hours, about 6 hours, about 12 hours, about 18 hours, about 24 hours, about 30 hours, about 36 hours, about 42 hours, about 48 hours, about 60 hours, about 72 hours, about 84 hours, or about 96 hours.
86. The method of Enumerated Example 83, wherein said heat stress comprises storing said sample at from about 25° C. to about 60° C., from about 30° C. to about 60° C., from about 35° C. to about 55° C., from about 40° C. to about 50° C., from about 44° C. to about 46° C., about 25° C., about 30° C., about 35° C., about 40° C., about 45° C., about 50° C., about 55° C., or about 60° C.
87. The method of Enumerated Example 86, wherein said heat stress comprises storing said sample at said temperature from 1 day to 6 months, from 3 days to 3 months, from 1 week to 2 months, from 0.5 months to 1 month, about 1 day, about 2 days, about 3 days, about 1 week, about 2 weeks, about 0.5 months, about 3 weeks, about 4 weeks, about 1 month, about 5 weeks, about 6 weeks, about 7 weeks, about 8 weeks, about 2 months, about 3 months, about 3.5 months, about 4 months, about 4.5 months, about 5 months, about 5.5 months, or about 6 months.
88. The method of any one of Enumerated Examples 82 to 87, wherein said purification step comprises filtration, enrichment, or chromatographic separation.
89. The method of Enumerated Example 88, wherein said chromatographic separation comprises cation exchange chromatography.
90. The method of Enumerated Example 88, wherein said chromatographic separation comprises size exclusion chromatography.
91. The method of any one of Enumerated Examples 82 to 90, wherein said HMW-depleted sample comprises less than 10%, less than 5%, less than 4%, less than 3%, less than 2%, or less than 1% HMW species.
92. The method of any one of Enumerated Examples 82 to 91, wherein said formulating step further comprises adding excipients to said HMW-depleted sample.
93. The method of any one of Enumerated Examples 82 to 92, wherein said fatty acid ester is polysorbate 20, polysorbate 40, polysorbate 60, or polysorbate 80.
94. The method of any one of Enumerated Examples 82 to 93, wherein said period of storage is from about 2 weeks to about 5 years, from about 2 weeks to about 3 years, from about 2 weeks to about 1 year, from about 2 weeks to about 6 months, from about 1 month to about 5 years, from about 1 month to about 3 years, from about 1 month to about 1 year, from about 1 month to about 6 months, about 2 weeks, about 3 weeks, about 4 weeks, about 1 month, about 2 months, about 3 months, about 4 months, about 5 months, about 6 months, about 9 months, about 12 months, about 18 months, about 2 years, about 3 years, about 4 years, or about 5 years.
95. The method of any one of Enumerated Examples 82 to 94, wherein said long-term storage conditions comprise storage at a temperature from about 0° C. to about 10° C., about 0° C., about 5° C., or about 10° C.
96. The method of any one of Enumerated Examples 82 to 95, wherein said accelerated storage conditions comprise storage at a temperature from about 35° C. to about 60° C., from about 45° C. to about 55° C., about 35° C., about 40° C., about 45° C., about 50° C., about 55° C., or about 60° C.
97. The method of any one of Enumerated Examples 82 to 96, wherein a measure of reduced lipase activity comprises a quantification of fatty acid ester degradation.
98. The method of Enumerated Example 97, wherein said quantification comprises an enzymatic colorimetric assay for non-esterified fatty acids, mass spectrometry, charged aerosol detection, and/or liquid chromatography.
99. The method of Enumerated Example 97, wherein said quantification comprises determining a percent of fatty acid ester recovered from said pharmaceutical composition with reduced lipase activity after said period of storage using charged aerosol detection-liquid chromatography.
100. The method of Enumerated Example 99, wherein said percent is above 80%, above 85%, above 90%, above 95%, above 99%, about 85%, about 90%, about 95%, about 99%, or about 100.
101. The method of any one of Enumerated Examples 82 to 100, wherein a measure of reduced lipase activity comprises a quantification of particles per unit volume.
102. The method of Enumerated Example 101, wherein said particles comprise visible particles and/or subvisible particles.
103. The method of Enumerated Example 101, wherein said particles comprise particles ≥10 μm in diameter and/or particles ≥25 μm in diameter.
104. The method of Enumerated Example 101, wherein said particles comprise fatty acid particles.
105. The method of any one of Enumerated Examples 101 to 104, wherein said quantification comprises light obscuration, optical microscopy, micro-flow image analysis, flow cytometry, Coulter counting, and/or submicron particle tracking.
106. The method of any one of Enumerated Examples 101 to 105, wherein a number of particles per mL in said pharmaceutical composition with reduced lipase activity is below 500, below 200, below 100, below 50, below 10, or below 5.
107. The method of any one of Enumerated Examples 82 to 106, wherein said dupilumab is produced from a recombinant host cell.
108. The method of Enumerated Example 107, wherein said recombinant host cell is selected from a group consisting of a CHO cell, a CHO-K1 cell, and variations thereof.
109. The method of any one of Enumerated Examples 82 to 108, wherein said lipase is a host cell protein.
110. The method of any one of Enumerated Examples 82 to 109, wherein said lipase has an increased susceptibility to inactivation in stress conditions compared to said dupilumab.
111. A protein formulation comprising dupilumab and at least one fatty acid ester made by the method of any one of Enumerated Examples 82 to 110.

What is claimed is:

1. A method for producing a recombinant protein, comprising:
(a) culturing cells expressing a recombinant protein in a seed train;
(b) transferring cells from each vessel of said seed train to the next vessel at a predetermined time point;
(c) transferring cells from the final vessel of said seed train to a production bioreactor at a predetermined time point; and
(d) isolating the recombinant protein from the production bioreactor.

2. The method of claim 1, wherein said recombinant protein is selected from a group consisting of an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, a variant thereof, a fragment thereof, and a multimer thereof.

3. The method of claim 1, wherein said recombinant protein is dupilumab.

4. The method of claim 1, wherein said final vessel is a 3,000 L bioreactor.

5. The method of claim 1, wherein the predetermined time point of step (c) is from 60 hours to 80 hours, from 65 hours to 75 hours, from 65 hours to 72.5 hours, from 67 hours to 70 hours, from 68 hours to 69 hours, or about 68.5 hours between inoculation of said final vessel and inoculation of said production bioreactor.

6. The method of claim 5, wherein said predetermined time point is about 68.5 hours.

7. The method of claim 1, wherein said seed train comprises a 500 L bioreactor at N-2.

8. The method of claim 7, wherein a predetermined time point for said 500 L bioreactor is from 50 to 52 hours or about 51 hours between inoculation of said 500 L bioreactor and inoculation of the next vessel in the seed train.

9. The method of claim 1, wherein said seed train comprises a 50 L bioreactor at N-3.

10. The method of claim 9, wherein a predetermined time point for said 50 L bioreactor is from 73 to 75 hours or about 74 hours between inoculation of said 50 L bioreactor and inoculation of the next vessel in the seed train.

11. The method of claim 1, wherein said seed train comprises a 20 L bioreactor at N-4.

12. The method of claim 11, wherein a predetermined time point for said 20 L bioreactor is from 73 to 75 hours or about 74 hours between inoculation of said 20 L bioreactor and inoculation of the next vessel in the seed train.

13. The method of claim 1, wherein said seed train comprises a 2 L bioreactor at N-5.

14. The method of claim 13, wherein a predetermined time point for said 2 L bioreactor is from 47.5 to 48.5 hours or about 48 hours between inoculation of said 2 L bioreactor and inoculation of the next vessel in the seed train.

15. The method of claim 1, wherein said seed train comprises a 500 mL shake flask at N-6.

16. The method of claim 15, wherein a predetermined time point for said 500 mL shake flask is from 79 to 80.5 hours or about 80 hours between inoculation of said 500 mL shake flask and inoculation of the next vessel in the seed train.

17. The method of claim 1, wherein said production bioreactor is a 10,000 L bioreactor.

18. The method of claim 1, wherein a titer of said recombinant protein is increased compared to a titer of a recombinant protein produced using a method wherein cells from each vessel of said seed train are transferred to the next vessel at a variable time point.

19. The method of claim 1, wherein said cells are selected from the group consisting of CHO cells, HEK293 cells, and BHK cells.

20. The method of claim 19, wherein said CHO cells are selected from the group consisting of CHO-K1, CHO DUX B-11, Veggie-CHO, GS-CHO, S-CHO, or CHO lec.

21. The method of claim 1, wherein said cells of step (c) have a viable cell density from 40-50×10⁵ viable cells/mL, from 45-50×10⁵ viable cells/mL, from 46-48×10⁵ viable cells/mL, from 46.5-47.5×10⁵ viable cells/mL, from 46.9-47.1×10⁵ viable cells/mL, about 40×10⁵ viable cells/mL, about 45×10⁵ viable cells/mL, about 47×10⁵ viable cells/mL, or about 50×10⁵ viable cells/mL.

22. The method of claim 1, further comprising determining said predetermined time points by selecting time points for each vessel of said seed train based on the achievement of optimized viable cell densities for each vessel of said seed train, wherein optimized viable cell densities are optimized for increased protein production from a production bioreactor.
